SEQUENCE LISTING (10 sequences)

SEQ ID NO: 1 - DNA, 1647 bp, Citrus sinensis
  Citrus valencene synthase
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaatagaagatgcaatacaaaaattatgtccaatctatattgacagtaatagagctgatctccacaccgtttcccttcattttcgattgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatacaataaaactctgctgaattttgcaaagttagattttaacatattgctagagctgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

SEQ ID NO: 2 - protein, 548 aa, Citrus sinensis
  Citrus valencene Synthase
MSSGETFRPTADFHPSLWRNHFLKGASDFKTVDHTATQERHEALKEEVRRMITDAEDKPVQKLRLIDEVQRLGVAYHFEKEIEDAIQKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLYNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 3 - protein, 548 aa, Citrus sinensis
  Citrus valencene synthase
MSSGETFRPTADFHPSLWRNHFLKGASDFKTVDHTATQERHEALKEEVRRMITDAEDKPVQKLRLIDEVQRLGVAYHFEKEIGDAIQKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLCNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 4 - protein, 548 aa, Citrus x paradisi
  citrus valencene synthase
MSSGETFRPTADFHPSLWRNHFLKGASDFKTVDHTATQERHEALKEEVRRMITDAEDKPVQKLRLIDEVQRLGVAYHFEKEIEDAILKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLYNKTLLNFAKLDFNILLEPHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 5 - protein, 548 aa, Citrus x paradisi
  Citrus valencene synthase
MSSGETFRPTADFHPSLWRNHFLKGASDFKTVDHTATQERHEALKEEVRRMITDAEDKPVQKLRLIDEVQRLGVAYHFEKEIEDAILKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLYNKTLLNFAKLDFNILLEPHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDIDEELMMKPTVVARPLLGTILNLARAIDFIYKEDDGYTHSYLIKDQIASVLGDHVPF

SEQ ID NO: 6 - DNA, 1647 bp, Citrus sinensis
  Citrus valencene synthase
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaataggagatgcaatacaaaaattatgtccaatctatattgacagtaatagagctgatctccacaccgtttcccttcattttcggttgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatgcaataaaactctgctgaattttgcaaagttagattttaacatattgctagagctgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

SEQ ID NO: 7 - DNA, 1647 bp, Citrus x paradisi
  Citrus valencene synthase
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaatagaagatgcaatactaaaattatgtccaatctatattgacagtaatagagctgatctccacaccgtttcccttcattttcgattgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatacaataaaactctgctgaattttgcaaagttagattttaacatattgctagagccgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

SEQ ID NO: 8 - DNA, 1877 bp, Citrus x paradisi
  Citrus valencene synthase
aatacaaattataattccacagaaatatcaaaattctgcggcacccgtttgtctgacacaaaaatgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaatagaagatgcaatactaaaattatgtccaatctatattgacagtaatagagctgatctccacaccgtttcccttcattttcgattgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatacaataaaactctgctgaattttgcaaagttagattttaacatattgctagagccgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattgacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttgacgactagttctatacttctattaataatattttcagttccttggtttgtgtttaaaggaaatacggtgtatcgtattggtctgtactaataaatattttatatttttattgttacttaaagattttcccttcgaataaaaaaataaactctaatataaaaaaaaaaa

SEQ ID NO: 9 - protein, 548 aa, Citrus paradisi
  Citrus valencene synthase
MSSGETFRPTADFHPSLWRNHFLKGASDFKTVDHTATQERHEALKEEVRRMITDAEDKPVQKLRLIDEVQRLGVAYHFEKEIEDAIQKLCPNYIHSNSPDLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLYNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 10 - DNA, 1647 bp, Citrus paradisi
  Citrus valencene synthase
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaatagaagatgcaatacaaaaattatgtccaaactatattcacagtaatagccctgatcttcacaccgtttctcttcattttcgattgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatacaataaaactctgctgaattttgcaaagttagattttaacatattgctagagctgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

(19) United States
(12) Patent Application Publication
(10) Pub. No.: US 2015/0250166 A1
(43) Pub. Date: Sep. 10, 2015

(54) NOOTKATONE AS AN INSECTICIDE AND INSECT REPELLENT

(71) Applicant: ALLYLIX, INC., San Diego, CA (US)

(72) Inventors: Seth Goldblum, San Diego, CA (US); Craig B. Warren, San Diego, CA (US)

(21) Appl. No.: 14/423,096

(22) PCT Filed: Aug. 21, 2013
(86) PCT No.: PCT/US2013/056060

§ 371 Date: Feb. 20, 2015

Related U.S. Application Data
(60) Provisional application No. 61/743,013, filed on Aug. 23, 2012.

(51) Int. Cl. A01N 35/06; A01N 43/20; A61K 8/35; A61K 31/122

(52) U.S. Cl. CPC A01N 35/06; A61K 31/122; A01N 43/20; A61K 8/35

(57) ABSTRACT

Pest control compositions and, in particular, pest repellent and pesticidal compositions containing nootkatone and/or a derivative or analog thereof, alone or combination with one or more active ingredients are provided. Methods of formulating and using the compositions are provided.

### RELATED APPLICATIONS

Benefit of priority is claimed to U.S. Provisional Application Ser. No. 61/743,013, filed Aug. 23, 2012, entitled “NOOTKATONE AS AN INSECTICIDE AND INSECT REPELLENT.” The subject matter of the above-noted application is incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 36 kilobytes in size, and titled 231SEQPC1.txt.

### FIELD OF THE INVENTION

Pest control compositions and, in particular, pest repellent and pesticidal compositions containing nootkatone and/or a derivative or analog thereof, alone or in combination with one or more active ingredients are provided. Methods of formulating and using the compositions are provided.

### BACKGROUND

Many insects are considered to be pests to animals, including humans, domesticated animals and pets because they often can serve as vectors for disease, can bite or sting, can cause annoyance, can damage property, including causing structural damage to homes or agricultural products, and can result in decreased enjoyment of indoor and outdoor environments. Among the insects often considered pest are ants, bedbugs, carpet beetles, centipedes, chiggers, drain flies, dust mites, earwigs, fleas, flies, gnats, hornets, lice, millipedes, mites, mosquitoes, scabies, silverfish, spiders, stinkbugs, termites, ticks, wasps, weevils and yellow jackets.

DEET (n-n-diethylnetatoluamide) is effective for repelling insects and nuisance pests. This compound does have its disadvantages. For example, DEET is perceived by many to have a strong ‘chemical’ smell at the concentrations usually used in most formulations. DEET is less effective in low concentrations.

In addition to DEET, examples of compounds used to repel or kills insect pests include organophosphates such as malathion, synthetic pyrethroids such as permethrin, carbamates and chlorinated hydrocarbons such as lindane. Synthetic chemical pesticides have been used to control insects and pests. Organic insecticides and inorganic salts are known in the art for their ability to repel or kill insects (e.g., see U.S. Pat. Nos. 2,423,284, 4,308,279, 4,376,784, 4,948,013, 5,434,189 and 6,048,892). Although some of these chemicals can be effective as pesticides when applied directly to insect pest, not all are approved for direct or indirect contact with animals, including humans.

Natural pesticides are considered safe to humans and the environment and have widespread use. Among the natural pest repellents or pesticides that are widely used are natural or synthetic oils of camphor, cedarwood, citronella, eucalyptus, pennyroyal, and the pyrethrins and pyrethroids. Plant oils tend to be expensive to isolate in commercial quantities and usually are very volatile, evaporating quickly when applied or exposed to the elements. Also, although initially these compounds were very successful as insect repellents and insecticides, there are reports that some pests are developing a resistance to them. For examples, it is reported that some bedbugs have developed a resistance to pyrethrins and pyrethroids. Resistance to insecticides in arthropods is widespread, with at least 400 species being resistant to one or more insecticides (U.S. Pat. No. 5,571,901).

In addition, some natural pesticides have unintended effects on animals exposed to them. Depending on the nature of the active ingredient, topical application may not be desirable as it can irritate the skin of the subject. For example, application of some pyrethrin to the skin can cause dermatitis, erythema, paresthesias (abnormal skin sensations) and rhinorrhea, and inhalation can result in asthma, headache, nausea, sneezing and vomiting (e.g., see U.S. Department of Labor, Chemical Sampling Information—Pyrethrin (2006)).

Accordingly, there is a need for pesticidal compositions for killing and/or repelling insect pests, particularly ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets, that do not require as ingredients any of the pyrethrins, synthetic pyrethroids, chlorinated hydrocarbons, organophosphates, or carbamates in order to be effective in killing or repelling insects, while at the same time the compositions are safe and effective. In addition, there is a need for methods for using such compositions, which are safe and non-toxic to humans, animals and the environment, for repelling insect pests, e.g., from the home, including from bedding, clothing and structures, and that are effective in obtaining acceptable levels of insect pest control, for the short term and when formulated for extended release for long-term insect pest control.

### SUMMARY

The compositions and methods provided herein satisfy these as well as other needs. It has been determined that the compound nootkatone is a very good repellent of insect pests, including ants, bedbugs, carpet beetles, centipedes, chiggers, drain flies, dust mites, earwigs, fleas, flies, gnats, hornets, lice, millipedes, mites (such as dust mites and itch mites), mosquitoes, roaches, silverfish, spiders, stinkbugs, termites, ticks, wasps, weevils and yellow jackets, and is very effective in killing insect pests including ants, bedbugs, carpet beetles, centipedes, chiggers, drain flies, dust mites, itch mites, earwigs, fleas, flies, gnats, hornets, lice, millipedes, mites, mosquitoes, scabies, silverfish, spiders, stinkbugs, termites, ticks, wasps, weevils and yellow jackets.

Provided are compositions including nootkatone or a derivative or analog thereof, generally in an amount of from 0.1% to 10% or greater, such as at least 10%, at least 20%, at least 25%, or at least 50%, and methods that include application of a composition containing nootkatone or a derivative or analog thereof, generally in an amount of from 0.1% to 10% or greater, such as at least 10%, at least 20%, at least 25%, or at least 50%, for repelling and/or killing insect pests. In some applications, the compositions are formulated to release an insect repelling/killing effective amount of nootkatone for a period of time selected from among at least 1 hour, 2 hours, 3 hours, 4 hours, 5 hours, 6, hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, at least 1 day, 2 days, 3 days, 4 days, 5 days, 6 days 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, at least 45 days, at least 60 days, at least 75 days, at least 90 days, at least 4 months, at least 5 months, at least 6 months, at least 7 months, at least 8 months, at least 9 months, at least 10 months, at least 11 months and at least 1 year. In some applications, the methods include application of pesticidal and/or pest repelling compositions that contain nootkatone, particularly those compositions that release an insect repelling/killing effective amount of nootkatone and/or a derivative or analog thereof over an extended period.

Compositions provided herein are safe, non-toxic pesticidal and pest repelling compositions based on nootkatone and/or analogs of nootkatone. Also provided are methods for killing and/or repelling insect pests using the compositions containing nootkatone and/or analogs of nootkatone.

Provided are pesticidal compositions and methods for treating, controlling, repelling, eradicating and/or killing pests, e.g., Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurysternus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp. and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Ornithonyssus sylviarum), poultry red mite (Dermanyssus gallinae), Trombicula spp., Leptotrombidium akamushi, Ornithodorus hermsi, Ornithodorus turicata, Ornithonyssus bacoti, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp. and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Werneckiella spp., Lepikentron spp., Trichodectes spp. and Felicola spp.

Also provided are pesticidal and pest repelling compositions containing nootkatone and/or a derivative or analog thereof and methods for repelling or killing bedbugs on a subject or in an environment occupied by a subject, such as a human or an animal, particularly humans and other mammals. Also provided are pesticidal or pest repelling compositions containing nootkatone and/or derivatives or analogs thereof and methods for repelling or killing pests, such as ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets.

Provided herein are compositions that contain nootkatone or an analog of nootkatone and a carrier for killing or repelling pests. Also provided are personal care or cosmetic compositions, household care compositions, cleansing compositions, insect repellents, insecticide or pesticide compositions, fabric treatment sheets, liquid fabric treatment compositions, fabric refresher spray compositions, moist towelette products, packaged pest repelling compositions and aerosol propellant pressurized sprayable pest repellents, each containing the provided compositions that include nootkatone and/or an analog of nootkatone for killing or repelling pests. Also provided are methods for repelling an insect or a pest from a location, methods for repelling bedbugs, methods of preventing skin injury due to biting insects or pests, methods for killing an insect or pest, methods for treating a structure infested with termites and methods for preventing infestation of termites in a wooden structure supported by a foundation, by applying or providing a composition that contains nootkatone and/or an analog of nootkatone for killing or repelling pests. Also provided herein are insect repellent compositions containing nootkatone and/or an analog of nootkatone for killing or repelling pests.

The compositions for killing or repelling pests provided herein can contain a carrier and at least 0.1%, or at least 1%, or at least 5%, or at least 7.5%, or at least 10%, or greater than 10%, or greater than 15%, or greater than 20%, or greater than 25%, or greater than 50% by weight nootkatone and/or a derivative or analog of nootkatone. In some applications, the nootkatone or analog of nootkatone or their combination can be present in an amount that is greater than 60%, 70%, 80%, 90%, 95% or 99% by weight of the composition. The derivative or analog of nootkatone in the provided compositions can be selected from among nootkatone-11,12-epoxide, nootkatone-1,10-epoxide, nootkatone-1,10-11,12-diepoxide, tetrahydro-nootkatone and 1,10-dihydronootkatone and combinations thereof. In one example, the provided compositions contain nootkatone in an amount at or about 0.01% to at or about 15% by weight of the composition. In another example, the compositions contain nootkatone or an analog of nootkatone in an amount of from at or about 1% to at or about 10% by weight of the composition. In another example, the compositions contain nootkatone or an analog of nootkatone in an amount of greater than 10% by weight of the composition. In another example, the compositions contain nootkatone and/or an analog of nootkatone in an amount of greater than 15% by weight of the composition. In another example, the compositions contain nootkatone and/or an analog of nootkatone in an amount of greater than 20% by weight of the composition. In another example, the compositions contain nootkatone and/or an analog of nootkatone in an amount of greater than 25% by weight of the composition. In another example, the compositions contain nootkatone or an analog of nootkatone or a combination thereof in an amount of up to 99% by weight of the composition.

For example, provided herein are compositions containing at least 1% nootkatone or an analog thereof; and an active ingredient selected from among N,N-diethyl-meta-toluamide (DEET), picaridin (2-(2-hydroxyethyl)-1-piperidinecarboxylic acid 1-methylpropyl ester), citronella oil, camphor oil, cedarwood oil, coumarin, 2-hydroxy-methylcyclohexyl acetic acid lactone, beta-alanine, 2-hydroxymethyl-cyclohexylidene acetic acid lactone, 2-hydroxy-methylcyclohexyl propionic acid lactone, p-menthane-3,8-diol, and 3-[N-butyl-N-acetyl]-aminopropionic acid ethyl ester and combinations thereof. In some examples, the active ingredient is present in an amount of from more than 0.1% to at or about 25%, by weight, of the composition.

In another example, provided herein are compositions containing at least 1% nootkatone or an analog thereof; and an active ingredient selected from among N,N-diethyl-meta-toluamide (DEET), picaridin (2-(2-hydroxyethyl)-1-piperidinecarboxylic acid 1-methylpropyl ester), citronella oil, camphor oil, cedarwood oil, coumarin, 2-hydroxy-methylcyclohexyl acetic acid lactone, beta-alanine, 2-hydroxymethyl-cyclohexylidene acetic acid lactone, 2-hydroxy-methylcyclohexyl propionic acid lactone, p-menthane-3,8-diol, and 3-[N-butyl-N-acetyl]-aminopropionic acid ethyl ester and combinations thereof for use for repelling biting insects. In some examples, the nootkatone is present in an amount of from more than 10% to at or about 25%, by weight of the composition.

In yet another example, provided herein are compositions for killing or repelling insects or pests, containing greater than 10%, by weight, nootkatone and/or a derivative or analog of nootkatone and a carrier. The derivative or analog of nootkatone in the provided compositions can be selected from among nootkatone-11,12-epoxide, nootkatone-1,10-epoxide, nootkatone-1,10-11,12-diepoxide, tetrahydro-nootkatone and 1,10-dihydronootkatone and combinations thereof. In one example, the provided compositions contain nootkatone in an amount of at or about 10.5% to at or about 25%, by weight, of the composition. In another example, the provided compositions contain nootkatone in an amount of from at or about 15% to at or about 20%, by weight, of the composition.

The provided compositions contain a carrier selected from among, but not limited to, water, an alcohol, an aldehyde, an alkane, an alkene, an amide, an amine, a diglyceride, an ester, an ether, a glycol ether, a fat, a fatty acid, a glycol ester, a ketone, lanolin, mineral oil, a monoglyceride, paraffin oil, a polyethylene glycol, petrolatum, a propylene carbonate, silicone, tall oils, a terpene hydrocarbon, a terpene alcohol, a triglyceride, finely divided organic solid material, finely divided inorganic solid materials and mixtures thereof. In one embodiment, the carrier can be an alcohol that is selected from among an aromatic alcohol, a C₁-C₆ monohydric alcohol, C₂-C₆ polyhydric alcohol, a polyvalent alcohol, and mixtures thereof. In one example, the carrier is an alcohol that is selected from among methanol, ethanol, propanol, butanol, sec-butanol, tert-butanol, and mixtures thereof. In a particular example, the carrier in the provided compositions is ethanol. In another particular example, the compositions contain a carrier that is isopropanol.

It has been determined that, for killing or repelling some insects or pests, formulations that do not contain isopropanol are advantageous. Thus, in some applications, the amount of isopropanol in the formulation does not exceed 5%, and in some formulations isopropanol is not included.

In some applications, the provided compositions contain a carrier that is an oil selected from among an almond oil, avocado oil, canola oil, cashew oil, cherry seed oil, cocoa butter, coconut oil, corn oil, cottonseed oil, flaxseed oil, grape seed oil, jojoba oil, macadamia nut oil, olive oil, palm oil, palm fruit oil, peanut oil, rapeseed oil, rice bran oil, safflower oil, sesame oil, soybean oil, sunflower oil, and walnut oil and combinations thereof.

In some applications, the carrier in the provided compositions can be a diethyl ether, isopropyl ether, n-propyl ether, or a combination thereof. In one example, the carrier is acetone, a methyl ketone, a methyl benzyl ketone, a methyl ethyl ketone, a methyl isopropyl ketone, a methyl butyl ketone, an ethyl ketone, benzyl methyl ketone, and combinations thereof. In some applications, the provided compositions contain a carrier that is ethylene glycol, ethylene glycol monomethyl ether, ethylene glycol dimethyl ether, methylene glycol, methylene glycol monomethyl ether, methylene glycol dimethyl ether, propylene glycol, propylene glycol monomethyl ether, propylene glycol dimethyl ether, butylene glycol, butylene glycol monomethyl ether, butylene glycol dimethyl ether and combinations thereof. In another example, the carrier is ethylene glycol, ethylene glycol monomethyl ether, ethylene glycol dimethyl ether, methylene glycol, methylene glycol monomethyl ether, methylene glycol dimethyl ether, propylene glycol, propylene glycol monomethyl ether, propylene glycol dimethyl ether, butylene glycol, butylene glycol monomethyl ether, butylene glycol dimethyl ether and combinations thereof.

In some applications, the carrier in the compositions provided herein is a finely divided organic solid material or finely divided inorganic solid material. In some examples, the carrier can be a dust, a granule, a powder or a salt crystal. In other examples, the carrier can be an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, vermiculite, and combinations thereof.

In some applications, the carrier in the compositions provided herein is an aerosol propellant that is selected from among argon, butane, carbon dioxide, a chlorofluorocarbon, dimethyl ether, a hydrocarbon, a hydrofluorocarbon, isobutane, nitrogen, propane, and a mixture thereof. In a particular example, the aerosol propellant contains difluoromethane, trifluoromethane, difluoroethane, trifluoroethane, tetrafluoroethane or octafluorocyclobutane or a combination thereof.

In some applications, the carrier in the compositions provided herein is a silicone oil that is selected from among cyclical silicones, linear or branched open chained silicones, and combinations thereof. In one example, the silicone oil is selected from among volatile silicones and non-volatile silicones. In another example, the silicone oil is a volatile silicone oil that is selected from among cyclic polydimethylsiloxanes containing an average of from about 3 to about 9 silicon atoms and linear polydimethylsiloxanes containing an average of from about 3 to about 9 silicon atoms. In another example, the silicone oil is a non-volatile silicone oil that is selected from among dimethicone copolyol, cyclomethicone, polydimethylsiloxane, cyclic dimethyl polysiloxane, aminosilicones, phenylsilicones, diphenyldimethicones, phenyltrimethicones, cyclopentasiloxane, a polymer of dimethyl-siloxane with polyoxyethylene and/or polyoxypropylene, dimethicone copolyol, cetyldimethicone copolyol, cetyl dimethicone, cetyl dimethicone copolyol and dimethiconol and combinations thereof. In further examples, the carrier is a silicone oil that is selected from among polydimethylsiloxane, phenylated silicones, decamethylcyclopentasiloxane, dodecamethylcyclohexasiloxane and octamethylcyclo-tetrasiloxane.

In some applications, the carrier in the compositions provided herein is selected from, but not limited to, a monoglyceride, a diglyceride, an acetylated monoglyceride, or a triglyceride or a combination thereof. In one example, the carrier contains 1-propanol, 2-propanol, 1-butanol, 2-butanol, 2-methyl-1-propanol, 1-pentanol, 2-pentanol, 3-methyl-1-butanol, 3-methyl-2-butanol, ethylene glycol, propylene glycol, 1,4-butanediol, 1,3-butanediol, 2-methyl-1,3-propanediol, 1,4-cyclohexanedimethanol, diethylene glycol, triethylene glycol, PEG-200, PEG-300, PEG-400, PEG-600, 2-methoxyethanol, 2-ethoxyethanol, 2-propoxyethanol, 2-isopropoxyethanol, 2-butoxyethanol, 1-methoxy-2-propanol, 1-ethoxy-2-propanol, 3-methoxy-1-butanol, diethylene glycol monomethyl ether, diethylene glycol monoethyl ether, diethylene glycol mono-n-propyl ether, diethylene glycol mono-isopropyl ether, diethylene glycol monobutyl ether, triethylene glycol monomethyl ether, glycerol, 3-methoxy-1,2-propanediol, or 3-ethoxy-1,2-propanediol.

In another example, the carrier in the provided compositions contains borneol, citronellol or geraniol or a combination thereof. In some applications, the carrier in the compositions provided herein contains or is a cyclodextrin that is selected from among an α-cyclodextrin, β-cyclodextrin or γ-cyclodextrin or a combinations thereof.

In some applications, the compositions provided herein can further contain a dispersing agent that is selected from among, but not limited to, a surfactant, polyvinyl pyrrolidone, polyoxyethylated castor oil, a polyoxyethylene sorbitan ester, alkylnaphthalene sulfonate, alkylbenzenesulfonate, polyoxyethylene, polycarboxylate, lignin sulfonate, sodium silicate, potassium silicate, methylcellulose, carboxymethyl cellulose, hydroxypropylcellulose, hydroxypropyl-methylcellulose, gum arabic, a polyacrylate, and an acrylic/maleic copolymers and combinations thereof. In one example, the dispersing agent is a surfactant that is selected from among an anionic surfactant, a cationic, a non-ionic surfactant, and a zwitterionic surfactant and a combination thereof. In a particular example, the surfactant is an anionic surfactant that is selected from among fatty soaps, alkyl sulfates, sulfated oils, ether sulfates, sulfonates, sulfosuccinates, sulfonated amides and isethionates. In another example, the anionic surfactant is selected from among alkyl sulfonate surfactants, a linear alkylbenzene sulfonic acid, a branched alkylbenzene sulfonic acid a C₁₂ to C₁₈ alkylsulfate, C₁₂-C₁₈ alkyl alkoxy sulfate, C₁₂-C₁₈ alkyl methyl ester sulfonate and combinations thereof. In a particular example, the surfactant is a cationic surfactant that is selected from among an alkylamine, an alkyl diamine, an alkyl polyamine, a mono- or di-quaternary ammonium salt, a monoalkoxylated amine, a dialkoxylated amine, a monoalkoxylated quaternary ammonium salt, a dialkoxylated quaternary ammonium salt, an etheramine, an amine oxide, an alkoxylated amine oxide and a fatty imidazoline and combinations thereof. In another particular example, the surfactant is a non-ionic surfactant that is selected from among an alkoxylated alcohol, a dialkoxylated alcohol, an alkoxylated dialkylphenol, an alkylpolyglycoside, an alkoxylated alkylphenol, an alkoxylated glycol, an alkoxylated mercaptan, an alkylamine salt, an alkyl quaternary amine salt, a glyceryl or polyglyceryl ester of a natural fatty acid, an alkoxylated glycol ester, an alkoxylated fatty acid, an alkoxylated alkanolamide, a polyalkoxylated silicone and an N-alkyl pyrrolidone and combinations thereof.

The dispersing agent can be present in the provided compositions in an amount of at or about 0.002% to at or about 50% by weight of the composition. In some examples, the dispersing agent is present in an amount of at or about 0.025% to at or about 25% by weight of the composition. In other examples, the dispersing agent is present in an amount of at or about 0.01% to at or about 15% by weight of the composition. In yet other examples, the dispersing agent is present in an amount of at or about 0.05% to at or about 10% by weight of the composition, for example, in an amount at or about at least 0.05%, 0.06%, 0.07%, 008%, 0.09%, 0.1%, 0.15%, 0.2%, 0.25%, 0.3%, 0.35%, 0.3%, 0.35%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95% and 10% by weight of the composition. In one example, the ratio of nootkatone or analog of nootkatone to dispersing agent in the provided compositions is at or about 5:1 (w/w) to at or about 1:50 (w/w).

In some applications, the compositions provided herein can further contain a viscosity modulating agent that is selected from among, but not limited to, an acrylate, an acrylate copolymer, an alginate, an arabinogalactan, a carrageenan, a cellulosic polymer, a ceramide, chitan, dextran, diutan, fucelleran, fucoidan, a β-glucan, a gellan gum, guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan, starch, starch hydroxyethyl ether, starch dextrins and a xanthan gum and combinations thereof. In one example, the viscosity modulating agent is a xanthan gum that is a low acetate xanthan gum or a high pyruvate xanthan gum. In another example, the viscosity modulating agent is a cellulosic polymer that is selected from among bacterial cellulose, carboxymethyl cellulose, ethyl cellulose, ethyl-hydroxyethylcellulose, hydroxylethyl cellulose, hydroxypropyl cellulose, hydroxypropyl methylcellulose, methyl cellulose, microparticulate cellulose and sodium carboxymethyl cellulose and combinations thereof. The viscosity modulating agent can be present in the provided compositions in an amount of at or about 0.05% to at or about 25% by weight of the composition. In one example, the viscosity modulating agent is present in an amount of at or about 0.1% to at or about 10% by weight of the composition. In another example, the viscosity modulating agent is present in an amount of at or about 0.5% to at or about 5% by weight of the composition.

In some applications, the compositions provided herein can further contain a gelling agent that is selected from among, but not limited to, agar, a carbomer, carboxyvinyl polymers, dibenzylidene alditols, collagen, dextrin fatty acid esters, gelatin, hydrogenated styrene/isoprene copolymers, 12-hydroxystearic acid, i-carrageenan, gellan gum, pectin, polyacrylic acids, styrene-ethylene/propylene block copolymers, styrene-ethylene/butylene-styrene block copolymers, sucrose fatty acid esters and a wax and combinations thereof. In some examples, the gelling agent is a wax that is selected from among candelilla wax, carnauba wax, ceresin wax, microcrystalline wax paraffin wax and polyethylene wax. In one example, the gelling agent is present in an amount of at or about 0.01% to at or about 10% by weight of the composition. In another example, the gelling agent is present in an amount of at or about 0.05% to at or about 7.5% by weight of the composition. In a further example, the gelling agent is present in an amount of at or about 0.1% to at or about 5% by weight of the composition. In yet another example, gelling agent is present in an amount of at or about 0.25% to at or about 2.5% by weight of the composition.

In some applications, the compositions provided herein can further contain an antioxidant that is selected from among, but not limited to, ascorbyl palmitate, butylated p-cresol, tert-butylhydroquinone, butylated hydroquinone monomethyl ether, butylhydroxy-anisole, butylhydroxytoluene, propyl gallate and a tocopherol and combinations thereof. In one example, the antioxidant is present in an amount of at or about 0.001% to at or about 5% by weight of the composition. In another example, the antioxidant is present in an amount of at or about 0.005% to at or about 2.5% by weight of the composition. In a further example, the antioxidant is present in an amount of at or about 0.01% to at or about 1% by weight of the composition.

In some applications, the compositions provided herein can further contain a preservative that is selected from among, but not limited to, an azole, benzisothiazolin-3-one, benzalkonium quaternary compounds, benzyl alcohol, borates, 2-bromo-2-nitro-propane-1,3-diol, butylparaben, 5-chloro-2-methyl-4-isothiazolin-3-one, chlorphenesin, chlor-oxylenol, diazolidinyl urea, a dimethyl-benzylalkyl-ammonium chloride, ethyl paraben, formaldehyde, glutaraldehyde, halogenated salicylanilides, hexachlorophene, isobutyl-paraben, isothiazolin-3-one, 2-methyl-4-isothiazoline-3-one, methylparaben, mono-chloracetamide, neomycin sulfate, o-phenylphenol and salts thereof, phenoxyethanol, propionic acid and salts thereof, propylparaben, sodium benzoate, sorbic acid and salts thereof, tebuconazole and triazoles, and combinations thereof. In one example, the preservative is present in an amount of at or about 0.001% to at or about 5% by weight of the composition. In another example, the preservative is present in an amount of at or about 0.005% to at or about 2.5% by weight of the composition. In yet another example, the preservative is present in an amount of at or about 0.01% to at or about 1% by weight of the composition.

In some applications, the compositions provided herein can further contain a colorant that is selected from among, but not limited to, a dye or pigment. In one example, the colorant is present in an amount at or about 0.0001% to at or about 1% by weight of the composition. In another example, the colorant is present in an amount at or about 0.0005% to at or about 0.5% by weight of the composition.

In some examples, the composition is formulated for delivery of nootkatone and/or a derivative or analog thereof for a period of time selected from among at least 1 hour, 2 hours, 3 hours, 4 hours, 5 hours, 6, hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, at least 1 day, 2 days, 3 days, 4 days, 5 days, 6 days 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, at least 45 days, at least 60 days, at least 75 days, at least 90 days, at least 4 months, at least 5 months, at least 6 months, at least 7 months, at least 8 months, at least 9 months, at least 10 months, at least 11 months and at least 1 year.

The compositions for killing or repelling pests provided herein can be formulated as a personal care or cosmetic composition. In some applications, the personal care or cosmetic composition is formulated as a product selected from among insect repellents, skin care products, hair care products, and cleansing products. In some examples, the personal care or cosmetic composition is a skin care product that is selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products and anti-wrinkle products. In other examples, the personal care or cosmetic composition is a hair care product that is selected from among hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays. In other examples, the personal care or cosmetic composition is a cleansing product that is selected from among soaps, foaming bath products, hand/body/facial cleansers, astringent cleansers, anti-acne products, shampoos, body shampoos, synthetic detergent bars, shower gels and shampoos.

The compositions for killing or repelling pests provided herein can be formulated as a household care composition. In some examples, the household care composition is formulated as a product that is selected from among air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles. In one example, the household care composition is a fabric softening composition that is selected from among a liquid fabric softener, a fabric softening rinse, a fabric softening sheet, and a fabric softening gel.

The compositions for killing or repelling pests provided herein can be provided in a form selected from among an aerosol, a bar, a cream, a gel, a liquid, a lotion, a paste, a powder, a roll-on, a sheet, a spray, a stick and a tablet form.

Also provided herein are cleansing compositions containing a composition for killing or repelling pests provided herein and from at or about 1% to at about 80% by weight of the composition of a detergent component. The detergent component can be selected from among anionic surfactants, nonionic surfactants, zwitterionic surfactants, ampholytic surfactants and cationic surfactants and mixtures thereof. The cleansing compositions can further contain a detergency builder component selected from among alkali metal carbonates, alkali metal phosphates, alkali metal phosphonates, alkali metal polyphosphates, alkali metal polyphosphonic acids, alkali metal silicates, C₈-C₁₈ alkyl monocarboxylic acids, alkali metal, ammonium or substituted ammonium salts of polycarboxylic acids and a zeolite and mixtures thereof in an amount of at or about 1% to at or about 80% by weight of the composition. The cleansing compositions can be provided in liquid form or can be provided in solid or powdered form. In some examples, the provided cleansing compositions are formulated as a heavy-duty detergent powder, heavy-duty detergent liquid, dishwashing liquid, machine dishwashing detergents, institutional detergents, detergent liquids, laundry aid, pretreatment aid, after-treatment aids, presoaking product, hard surface cleaner, or carpet cleaner.

Also provided herein are insect repellents containing a composition for killing or repelling pests provided herein and a delivery vehicle to deliver the insect repellent to a selected location. The delivery vehicle can contain water, an aerosol, a cream, a gel, a lotion, an oil, a spray, a soap, a detergent, a particulate or a substrate. In some examples, the composition in the insect repellent contains at least 1% nootkatone or analog of nootkatone. In other examples, the composition in the insect repellent contains at or about 1% to at or about 10%, or greater than 10%, or greater than 15%, or greater than 20%, or greater than 25% nootkatone or analog of nootkatone. In some applications, the delivery vehicle in the insect repellents can be a substrate that is a paper, a cloth or a woven or nonwoven material. In one example, the substrate is a nonwoven material that is a flexible sheet containing fibers that are adhesively or thermally bonded. The fibers can be selected from among cellulose ester, cotton, hemp, jute, linen, ramie, rayon, polyamides, polyesters polyolefins, polypropylene, polyvinyl derivatives, silk, sisal and wool and combinations thereof.

In some applications, the delivery vehicle in the insect repellents can be a gel that contains 0.2 to 5% of a gelling agent that is selected from among agar, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. In some applications, the delivery vehicle in the insect repellent is a fluid that when dispensed forms a gel in situ. The fluid can contain 0.2 to 5% of a gelling agent that selected from among agar, an alginate, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof.

In some applications, the delivery vehicle in the insect repellent can be or contain a particulate that is selected from among an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, and vermiculite, and combinations thereof.

In some examples, the insect repelled by an insect repellent provided herein can be an insect selected from among Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurysternus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp. and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Ornithonyssus sylviarum), poultry red mite (Dermanyssus gallinae), Trombicula spp., Leptotrombidium akamushi, Ornithodorus hermsi, Ornithodorus turicata, Ornithonyssus bacoti, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp. and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Werneckiella spp., Lepikentron spp., Trichodectes spp. and Felicola spp.

In other examples, the insect or pest repelled by an insect repellent provided herein can be an insect selected from among ants, bedbugs, carpet beetles, centipedes, chiggers, drain flies, dust mites, biting mites, earwigs, fleas, flies, gnats, hornets, lice, millipedes, mites, mosquitoes, roaches, scabies, silverfish, spiders, stinkbugs, termites, ticks, wasps, weevils and yellow jackets. The insect repellents provided herein can be formulated for delivery of nootkatone or analog of nootkatone for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days.

Also provided herein are an insecticide or pesticide compositions containing nootkatone and/or an analog of nootkatone that include a carrier selected to adhere to the insect or pest or to penetrate the exoskeleton of the insect or pest. The nootkatone and/or analog of nootkatone in the insecticide or pesticide composition can be present in an amount of at or about 0.1% to at or about 10%, or greater than 10%, or greater than 15%, or greater than 20% or greater than 25% by weight. The nootkatone and/or analog of nootkatone in the composition can be present in an amount of up to 99% by weight of the composition. In some examples, the carrier contains a particulate selected from among an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, and vermiculite, and combinations thereof. In other examples, the carrier forms a viscous fluid or gel when dispensed. For example, the carrier contains from 0.2 to 5% of a gelling agent that is selected from among agar, an alginate, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. In yet other examples, the carrier contains 0.2 to 20% of a viscosity modulating agent that is selected from among an acrylate, an acrylate copolymer, an alginate, an arabinogalactan, a carrageenan, a cellulosic polymer, a ceramide, chitan, dextran, diutan, fucelleran, fucoidan, a β-glucan, a gellan gum, guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan, starch, starch hydroxyethyl ether, starch dextrins and a xanthan gum and combinations thereof. The insecticide or pesticide compositions provided herein can further contain silicone dioxide, petroleum distillate, light solvent naphtha or D-limonene or combinations thereof.

Also provided herein are fabric treatment sheets containing a woven or nonwoven sheet or cellulosic substrate that is coated or impregnated with a fabric treatment composition and a composition for killing or repelling pests provided herein that contains nootkatone and/or an analog of nootkatone. The fabric treatment composition can be a detergent composition or a fabric softening composition. In some examples, the fabric treatment composition is a detergent composition that contains a detergent selected from among anionic surfactants, nonionic surfactants, zwitterionic surfactants, ampholytic surfactants and cationic surfactants and mixtures thereof. In other examples, the fabric treatment composition is a fabric softening composition that contains monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries or (C₈-C₂₄) fatty acid amides or any combination thereof.

Also provided herein are liquid fabric treatment compositions containing a fabric softener or fabric conditioner and a composition for killing or repelling pests provided herein. The fabric softener can be selected from among monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries and (C₈-C₂₄) fatty acid amides and combinations thereof. In some examples, the fabric conditioner contains an anti-static agent, a brightening agent, a bodying agent, a soil-release agent, a wrinkle-release agent or a combination thereof. In a particular example, the fabric condition is an anti-static agent that contains a tertiary amine, a quaternary amine, aluminum stearate or a combination thereof. In another example, the fabric conditioner is a brightening agent that contains hydrogen peroxide, potassium permanganate, sodium peroxide, sodium perborate, disulfonated diaminostilbene optical brightener compounds and triazole optical brightener compounds. In yet another example, the fabric conditioner is a bodying agent that is selected from among carboxymethyl cellulose, hydroxyethylcellulose, starch, polyvinyl acetate and combinations thereof. In yet another example, the fabric conditioner is a wrinkle release agent that contains polyvinyl acetate.

Also provided herein are fabric refresher spray compositions containing a composition for killing or repelling pests provided herein wherein the nootkatone or analog of nootkatone is present at a concentration of from at or about 0.1% to at or about 5%, or in an amount of at or about 1% to at or about 10%, or greater than 10%, or greater than 15%, or greater than 20% or greater than 25% by weight of the composition. The fabric refresher spray compositions can further contain a cyclodextrin; and/or an ampholytic surfactant, an anionic surfactant, a cationic surfactant, a nonionic surfactant or a zwitterionic surfactant or a combination thereof; and/or water.

Also provided herein are moist towelette products containing a substrate and a solution or emulsion of a composition for killing or repelling pests provided herein wherein the nootkatone or analog of nootkatone is present in an amount of at least 0.1% by weight of the solution. The nootkatone or analog of nootkatone can be present in an amount greater than 0.5%, or greater than 1%, or greater than 5%, or greater than 10%, or greater than 15%, or greater than 20%, or greater than 25% by weight of the solution. For example, the composition for killing or repelling pests can contain at or about at least 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, 10%, 10.05%, 10.1%, 10.15%, 10.2%, 10.25%, 10.3%, 10.35%, 10.4%, 10.45%, 10.5%, 10.55%, 10.6%, 10.65%, 10.7%, 10.75%, 10.8%, 10.85%, 10.9%, 10.95%, 11%, 11.05%, 11.1%, 11.15%, 11.2%, 11.25%, 11.3%, 11.35%, 11.4%, 11.45%, 11.5%, 11.55%, 11.6%, 11.65%, 11.7%, 11.75%, 11.8%, 11.85%, 11.9%, 11.95%, 12%, 12.05%, 12.1%, 12.15%, 12.2%, 12.25%, 12.3%, 12.35%, 12.4%, 12.45%, 12.5%, 12.55%, 12.6%, 12.65%, 12.7%, 12.75%, 12.8%, 12.85%, 12.9%, 12.95%, 13%, 13.05%, 13.1%, 13.15%, 13.2%, 13.25%, 13.3%, 13.35%, 13.4%, 13.45%, 13.5%, 13.55%, 13.6%, 13.65%, 13.7%, 13.75%, 13.8%, 13.85%, 13.9%, 13.95%, 14%, 14.05%, 14.1%, 14.15%, 14.2%, 14.25%, 14.3%, 14.35%, 14.4%, 14.45%, 14.5%, 14.55%, 14.6%, 14.65%, 14.7%, 14.75%, 14.8%, 14.85%, 14.9%, 14.95%, 15%, 15.05%, 15.1%, 15.15%, 15.2%, 15.25%, 15.3%, 15.35%, 15.4%, 15.45%, 15.5%, 15.55%, 15.6%, 15.65%, 15.7%, 15.75%, 15.8%, 15.85%, 15.9%, 15.95%, 16%, 16.05%, 16.1%, 16.15%, 16.2%, 16.25%, 16.3%, 16.35%, 16.4%, 16.45%, 16.5%, 16.55%, 16.6%, 16.65%, 16.7%, 16.75%, 16.8%, 16.85%, 16.9%, 16.95%, 17%, 17.05%, 17.1%, 17.15%, 17.2%, 17.25%, 17.3%, 17.35%, 17.4%, 17.45%, 17.5%, 17.55%, 17.6%, 17.65%, 17.7%, 17.75%, 17.8%, 17.85%, 17.9%, 17.95%, 18%, 18.05%, 18.1%, 18.15%, 18.2%, 18.25%, 18.3%, 18.35%, 18.4%, 18.45%, 18.5%, 18.55%, 18.6%, 18.65%, 18.7%, 18.75%, 18.8%, 18.85%, 18.9%, 18.95%, 19%, 19.05%, 19.1%, 19.15%, 19.2%, 19.25%, 19.3%, 19.35%, 19.4%, 19.45%, 19.5%, 19.55%, 19.6%, 19.65%, 19.7%, 19.75%, 19.8%, 19.85%, 19.9%, 19.95% or 20% nootkatone or analog of nootkatone. The moist towelette products can further contain a surfactant containing, but not limited to, cocamidopropyl betaine, coco-glucoside or decyl glucoside or combinations thereof. In one example, the substrate in the moist towelette product is a nonwoven fabric or cellulosic material.

Also provided herein are packaged pest repelling compositions that contain a container holding a composition for killing or repelling pests provided herein or an absorbent sheet impregnated with a composition for killing or repelling pests provided herein. Also provided herein are aerosol propellant pressurized sprayable pest repellents or pesticide products containing a composition for killing or repelling pests provided herein and at least at or about 5% to 75% propellant by weight of the composition. The propellant in the aerosol propellant pressurized sprayable pest repellents or pesticide products can contain carbon dioxide, propane, butane or a mixture thereof.

Provided herein are methods of repelling an insect or pest from a location using a composition for killing or repelling pests provided herein. In the provided methods, a composition for killing or repelling pests provided herein that contains from at or about 0.1% to at or about 10%, or greater than 10%, or greater than 15%, or greater than 10%, or greater than 25% nootkatone or analog of nootkatone by weight of the composition, is provided and deployed at the location in an insect or pest repelling amount, wherein the insect or pest is repelled when the insect or pest comes into contact with the composition or vapors from the composition. In the provided methods, the composition can be deployed by atomizing, brushing on, coating, dipping, drenching, dripping, dusting, foaming, infusing, injecting into or onto, pouring, rolling on, scattering, spraying, spreading, sprinkling or wiping the composition onto at least a portion of the location. In some examples in the methods provided herein, the composition contains from at or about 0.1% to at or about 10%, or greater than 10%, or greater than 15%, or greater than 20%, or greater than 25% nootkatone and/or analog or derivative of nootkatone, for example, at least 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9% or 20% nootkatone or analog or derivative of nootkatone based on the weight of the composition. In some applications, the nootkatone or of nootkatone or their combination is present in an amount of up to 99% by weight of the composition.

In some examples of the method for repelling an insect or pest from a location, the location is the surface of the body of a human or animal. For example, the animal can be a companion animal or a farm animal. In other examples of the method, the composition is deployed by applying topically to an article of clothing of a human. In other examples of the method, the composition is deployed by laundering an article of clothing of a human with a detergent or fabric softener or both that contains the composition. In yet another example of the method, the composition is deployed by drying an article of clothing of a human with a fabric softener that contains the composition. The fabric softener can be provided as a dryer sheet or gel. In another example of the method, the location is a surface that is skin, hair or fur and the composition is deployed by applying topically to the skin, hair or fur. The topically applied composition can be provided as an aerosol, a solution, an emulsion, an oil, a lotion, a soap, a spray, or a gel. In yet another example of the method, the composition is provided to a surface that is skin, hair or fur in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos.

In some examples of the method for repelling an insect or pest from a location, the location is a bedding location. The composition can be deployed onto bedding, bed boards, bed slats, a mattress, box springs, furniture, carpeting, baseboards or flooring or a combination thereof by atomizing, coating, dipping, drenching, dripping, dusting, foaming, infusing, injecting into or onto, pouring, rolling on, scattering, spraying, spreading, sprinkling or wiping. In another aspect of the method, the composition is deployed by spraying the composition on to the surface of bedding, bed boards, bed slats, a mattress, box springs, furniture, carpeting, baseboards or flooring or a combination thereof. For example, the composition can be deployed by injecting the composition into the mattress, box springs, furniture, carpeting, baseboards or flooring or a combination thereof. In some examples of the method, the contains a wood structure, wooden object or wall space. In other examples of the method, the location is selected from among an air supply duct, an attic, an awning, a basement, a cellar, a crawlspace, a deck, a dock, a garage, a hamper, a heating vent, a home foundation, a linen storage closet, a pool deck, roof tiles, a shipping container, a storage unit, a suitcase, a walkway and a wall space and the composition is deployed by atomizing, coating, dipping, drenching, dripping, dusting, foaming, infusing, injecting into or onto, pouring, rolling on, scattering, spraying, spreading, sprinkling or wiping the composition onto or into at least a portion of the location. For example, the composition is applied by spraying the composition onto a surface or is provided in the form of a powder and is applied by sprinkling the powder composition onto a surface. In yet other examples of the method for repelling an insect or pest from a location the composition is deployed by providing an absorbent substrate or gel containing the composition and positioning it in the location. In one example, the absorbent substrate contains a nonwoven fabric or cellulosic material.

In further examples of the provided method for repelling an insect or pest from a location, the composition contains a carrier and from at or about 0.1% to at or about 10% nootkatone or analog of nootkatone, or at least 10%, or at least 15%, or at least 20%, or at least 25% nootkatone and/or analog of nootkatone. In some examples of the method, the composition is formulated for delivery of nootkatone or analog of nootkatone for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days. In the provided method, the insect or pest can be selected from among, but not limited to, ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets. For example, in the provided method, the insects or pests are ants that are selected from among Argentine ants, black ants, carpenter ants, fire ants, odorous house ants, pavement ants and pharaoh ants. In another example in the provided method, the insects or pests are lice that are selected from among head lice, body lice, pubic lice and nits thereof.

Also provided herein is a method for repelling insects or pests by deploying a composition for killing or repelling insects or pests provided herein that contains from at or about 0.01% to at or about 10%, or greater than 10%, or greater than 15%, or greater than 20%, or greater than 25% nootkatone and/or analog of nootkatone; and the insect or pest is repelled when the insect or pest comes into contact with the composition or vapors from the composition. In the provided method, the composition can be deployed by applying topically to an article of clothing of a human; or applying topically to skin or hair of a human; or applying topically to skin or fur of an animal. In some examples of the method, the animal can be selected from among a bovine, canine, caprine, cervine, cricetine, feline, galline, equine, lapine, murine, musteline and ovine. For example, the animal is a companion animal. In other examples of the provided method, the composition can be deployed by laundering an article of clothing of a human with a detergent or fabric softener or both that contains the composition; or drying an article of clothing of a human with a fabric softener that contains the composition.

Also provided are methods for repelling bedbugs, where a composition that contains from at or about 0.01% to at or about 10%, or greater than 10%, or greater than 15%, or greater than 20%, or greater than 25% nootkatone and/or analog of nootkatone is deployed by applying directly to the surface of the bedbugs or to bedding, bed boards, bed slats, a mattress, box springs, furniture, carpeting, baseboards or flooring or a combination thereof. For example, application of the composition can include spraying the composition on to the surface of bedding, bed boards, bed slats, a mattress, box springs, furniture or carpeting; or injecting the composition into the mattress, box springs, furniture or carpeting or a combination thereof; or deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or injecting the composition into a wall space. In a particular example of the provided method, the absorbent substrate contains a nonwoven fabric or cellulosic material.

In one example of the provided method for repelling bedbugs, the composition contains from at or about 0.01% to at or about 10%, or greater than 10%, or greater than 15%, or greater than 20%, or greater than 25%, or up to 99% nootkatone or analog of nootkatone and a carrier. In some examples, the composition is formulated for delivery of nootkatone or analog of nootkatone for at least 4 days. In any of the methods provided herein, the composition can be formulated for delivery of nootkatone or analog of nootkatone or a combination thereof for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days.

Also provided herein is a method of preventing skin injury due to biting insects or pests by providing a composition for killing or repelling insects provided herein that contains at or about 1% to at or about 10%, or greater than 10%, or greater than 15%, or greater than 20% or greater than 25%, or up to 99% nootkatone and/or analog of nootkatone by weight of the composition; and applying the composition to a surface, wherein the insect or pest is repelled from the surface when it comes into contact with the composition or with vapors from the composition. In some examples the surface is clothing or bedding and the composition can be applied to the surface by atomizing, coating, dipping, drenching, dripping, dusting, foaming, infusing, injecting into or onto, pouring, rolling on, scattering, spraying, spreading, sprinkling or wiping an amount of the composition onto the surface. In other examples of the provided method, the composition is applied by washing the clothing or bedding with the composition that is provided as a detergent composition or a fabric softener composition or both. In other examples of the provided method, the composition can be applied by drying the clothing or bedding with the composition that is provided as a fabric softener composition. In some examples of the method, the surface is bed boards, bed slats, a mattress, box springs, furniture or carpeting and the composition can be applied by atomizing, coating, dipping, drenching, dripping, dusting, foaming, infusing, injecting into or onto, pouring, rolling on, scattering, spraying, spreading, sprinkling or wiping the composition onto the surface. In yet another example of the method, the composition can be provided in the form of a powder and is applied by sprinkling the powder composition onto the surface.

In some examples of the method for preventing skin injury due to biting insects or pests, the surface is skin or hair of a human and the composition can be applied topically to the skin or hair. In this example, the composition can be provided as an aerosol, a solution, an emulsion, an oil, a lotion, a soap, a spray, or a gel. In yet other examples of the method, the composition is provided in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos. The insect or pest can be selected from ants, bedbugs, chiggers, fleas, lice, mites, mosquitoes, roaches, scabies, and ticks.

Also provided herein is a method of treating a fabric article to repel or kill bedbugs, whereby the method involves heating and tumbling in a dryer the fabric article in contact with flexible sheet substrate coated or impregnated with a composition for killing or repelling bedbugs provided herein.

Also provided herein is a method for killing an insect or pest by providing an insecticide formulation containing a composition for killing or repelling bedbugs provided herein, that contains at or about 0.1% to at or about 10%, or greater than 10%, or greater than 15%, or greater than 20%, or greater than 25%, or up to 99% nootkatone and/or analog of nootkatone by weight of the composition; and applying the composition to the insect or pest, whereby the insect or pest is killed. In this method the insecticide formulations further can further contain silicone dioxide, petroleum distillate, light solvent naphtha or D-limonene or combinations thereof. In some examples of the method, the insecticide formulation is formulated to form a viscous fluid or gel when dispensed and applied to the insect or pest. In other examples of the provided method, the insecticide formulation contains from 0.2 to 5% of a gelling agent selected from among agar, an alginate, a carbomer, carboxyvinyl polymers, dibenzylidene alditols, carboxy-polymethylene, collagen, dextrin fatty acid esters, gelatin, hydrogenated styrene/isoprene copolymers, 12-hydroxy-stearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. In other examples of the provided method, the insecticide formulation contains from 0.2 to 20% of a viscosity modulating agent selected from among an acrylate, an acrylate copolymer, an alginate, an arabinogalactan, a carrageenan, a cellulosic polymer, a ceramide, chitan, dextran, diutan, fucelleran, fucoidan, a β-glucan, a gellan gum, guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan, starch, starch hydroxyethyl ether, starch dextrins and a xanthan gum and combinations thereof. In the provided method, the insect or pest can be selected from among, but not limited to, Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurysternus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp. and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Ornithonyssus sylviarum), poultry red mite (Dermanyssus gallinae), Trombicula spp., Leptotrombidium akamushi, Ornithodorus hermsi, Ornithodorus turicata, Ornithonyssus bacoti, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp. and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Werneckiella spp., Lepikentron spp., Trichodectes spp. and Felicola spp.

Also provided herein is a method of treating a structure infested with termites by deploying a composition for killing or repelling bedbugs provided herein to the infested structure, wherein the composition kills or repels the termites. In some examples, the structure contains wood, a wood-based material, or combinations thereof. For example, the structure is a house or building. For example, the composition for use in the provided method contains a concentration of the nootkatone or analog or derivative thereof is between at or about 0.1% and at or about 10.0%, or greater than 10%, or greater than 15%, or greater than 20% or greater than 25% nootkatone and/or analog or derivative thereof based on the weight of the composition.

Also provided herein is a method for preventing infestation of termites in a wooden structure supported by a foundation, by moving the soil around at least a portion of the structure to expose at least a portion of the foundation; applying to the exposed foundation a composition for killing or repelling termites provided herein, the composition containing a concentration of nootkatone or analog or derivative thereof between at or about 0.1% and at or about 10.0%, or greater than 10%, or greater than 15%, or greater than 20% or greater than 25% nootkatone and/or analog or derivative thereof based on the weight of the composition; and replacing to soil to cover the exposed foundation; wherein the composition forms a barrier to deter migration of termites into the structure.

Also provided herein is a method for treating a subject infested with an insect or pest, by providing a composition for killing or repelling an insect or pest that contains at or about 0.1% to at or about 10%, or greater than 10%, or greater than 15%, or greater than 20% or greater than 25% nootkatone and/or analog of nootkatone by weight of the composition; and applying the composition to the subject, wherein the insect or pest is repelled from the surface or killed when it comes into contact with the composition or with vapors from the composition. In some examples of the method, the subject is an animal, for example, a human or a companion animal. In some examples of the method, the composition is applied to the skin, hair or fur. In other examples of the method, the insect or pest is selected from among chiggers, fleas, lice, mites and scabies.

Also provided herein is an insect repellent composition containing at or about 0.01% to at or about 10%, or 0.5% to 15%, or greater than 10%, or greater than 15%, or greater than 20%, or greater than 25%, or up to 99% nootkatone or analog of nootkatone by weight of the composition and an active ingredient selected from among N,N-diethyl-meta-toluamide (DEET), picaridin (2-(2-hydroxyethyl)-1-piperidinecarboxylic acid 1-methyl-propyl ester), citronella oil, camphor oil, cedarwood oil, coumarin, 2-hydroxymethyl-cyclohexyl acetic acid lactone, beta-alanine, 2-hydroxymethyl-cyclohexylidene acetic acid lactone, 2-hydroxy-methylcyclohexyl propionic acid lactone, p-menthane-3,8-diol, and 3-[N-butyl-N-acetyl]-aminopropionic acid ethyl ester and combinations thereof. In some examples, the composition contains the active ingredient in an amount of from at or about 0.1% to at or about 25% by weight of the composition. In other examples, the active ingredient contains DEET at a concentration of from at or about 2.5% to at or about 25% by weight of the composition. In further examples, the active ingredient contains DEET at a concentration of from at or about 2.5% to at or about 5% or from at or about 5% to at or about 15% or from at or about 10% to at or about 20% by weight of the composition.

Provided herein are methods for treating lice, wherein the method involves the steps of contacting an affected body part containing lice with a composition that contains at least 0.1% nootkatone or an analog thereof. The lice can be selected from among head lice, body lice, pubic lice and nits thereof. In some examples, the composition is formulated as a shampoo or body wash. In some examples, the composition contains at least 5% nootkatone. In other examples, the composition contains at least 10% or more than 10% nootkatone.

Provided herein is a composition for use for treating body lice, wherein the composition contains at least 5% nootkatone. In some examples, the composition contains at least 10% or more than 10% nootkatone. In some examples, the composition for use for treating body lice is formulated as a shampoo or body wash.

Provided herein is a method for treating a subject infested with chiggers or mites, wherein the method involves the steps of providing a composition containing at least 0.1% nootkatone or an analog thereof; and applying the composition to a surface of the subject, wherein the chiggers or mites are repelled from the surface or die after coming into contact with the composition or with vapors from the composition. In some examples of the method, the subject is a human or a companion animal and the surface of the subject is skin, hair or fur. In some examples of the method, the composition is provided in a form selected from among skin conditioners, hand/body/facial lotions, skin moisturizers, skin toners, skin sanitizers, skin cleansing compositions, skin soothing and lubricating compositions, sunscreen products, hair conditioners, hair styling gels, hair anti-dandruff compositions, hair lotions, hair tonics, rinses, conditioners, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars and shower gels.

### DETAILED DESCRIPTION

A. Definitions

B. Nootkatone

C. Methods of making or obtaining nootkatone

D. Valencene
1. Valencene Synthase
2. Production of Valencene

E. Assessment of Terpenes

F. Compositions
1. Carrier
   a. Liquid Carriers
   b. Gas Carriers
   c. Solid Carriers
2. Additional Ingredients
   a. Anti-Oxidants
   b. Emulsifiers and Dispersing Agents
   c. Viscosity Modulating Agents
   d. Preservatives
   e. Colorant
   f. Synergists
3. Microencapsulation

G. Formulations
1. Sprays
2. Dusts & Granules
3. Woven or Nonwoven Substrates
4. Aerosols
5. Personal Care & Cosmetic Formulations
6. Insect Repellents
7. Insecticides and Pesticides
8. Household Care Formulations

H. Preparation of the Compositions and Formulations

I. Methods

J. Examples

### A. DEFINITIONS

Unless defined otherwise, all technical and scientific terms used herein have the same meaning as is commonly understood by one of skill in the art to which the claimed subject matter belongs. All patents, patent applications or published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of subjects. Reactions and purification techniques can be performed, e.g., as commonly accomplished in the art or as described herein. The foregoing techniques and procedures can be generally performed using conventional methods well known in the art and as described in various general and more specific references that are cited and discussed throughout the present specification.

As used herein, nucleic acids or nucleic acid molecules include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule cannot be paired. Such unpaired ends will, in general, not exceed 20 nucleotides in length.

As used herein, heterologous nucleic acid is nucleic acid that is not normally produced in vivo by the cell in which it is expressed or that is produced by the cell but is at a different locus or expressed differently or that mediates or encodes mediators that alter expression of endogenous nucleic acid, such as DNA, by affecting transcription, translation, or other regulatable biochemical processes. Heterologous nucleic acid is generally not endogenous to the cell into which it is introduced, but has been obtained from another cell or prepared synthetically. Heterologous nucleic acid can be endogenous, but is nucleic acid that is expressed from a different locus or altered in its expression. Generally, although not necessarily, such nucleic acid encodes RNA and proteins that are not normally produced by the cell or in the same way in the cell in which it is expressed. Heterologous nucleic acid, such as DNA, also can be referred to as foreign nucleic acid, such as DNA. Thus, heterologous nucleic acid or foreign nucleic acid includes a nucleic acid molecule not present in the exact orientation or position as the counterpart nucleic acid molecule, such as DNA, is found in a genome. It also can refer to a nucleic acid molecule from another organism or species (i.e., exogenous).

Any nucleic acid, such as DNA, that one of skill in the art would recognize or consider as heterologous or foreign to the cell in which the nucleic acid is expressed is herein encompassed by heterologous nucleic acid; heterologous nucleic acid includes exogenously added nucleic acid that also is expressed endogenously. Examples of heterologous nucleic acid include, but are not limited to, nucleic acid that encodes traceable marker proteins, such as a protein that confers drug resistance, and nucleic acid, such as DNA, that encodes other types of proteins, such as antibodies. Antibodies that are encoded by heterologous nucleic acid can be secreted or expressed on the surface of the cell in which the heterologous nucleic acid has been introduced.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain). In keeping with standard polypeptide nomenclature described in J. Biol. Chem., 243: 3557-3559 (1968), and adopted 37 C.F.R. §§1.821-1.822, abbreviations for the amino acid residues that occur in the various sequences of amino acids provided herein are identified according to their known, three-letter or one-letter abbreviations as shown in Table 1:

TABLE 1
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, “non-natural amino acid” refers to an organic compound containing an amino group and a carboxylic acid group that is not one of the naturally-occurring amino acids listed in Table 1. Non-naturally occurring amino acids thus include, for example, amino acids or analogs of amino acids other than the 20 naturally-occurring amino acids and include, but are not limited to, the D-isostereomers of amino acids. Exemplary non-natural amino acids are known to those of skill in the art and can be included in a modified valencene synthase polypeptides provided herein.

Amino acid replacements or substitutions contemplated include conservative substitutions, including, but not limited to, those set forth in Table 2. Suitable conservative substitutions of amino acids are known to those of skill in the art and can be made generally without altering the conformation or activity of the polypeptide. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Conservative amino acid substitutions are made, for example, in accordance with those set forth in Table 2 as follows:

TABLE 2

| | Conservative |
|---|---|
| Original residue | substitution |
| Ala (A) | Gly; Ser; Abu |
| Arg (R) | Lys; orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met |

Other conservative substitutions also are permissible and can be determined empirically or in accord with known conservative substitutions. The effects of such substitutions can be calculated using substitution score matrices such PAM 120, PAM-200, and PAM-250 as discussed in Altschul, (J. Mol. Biol. 219:55565 (1991)).

As used herein, modification is in reference to modification of a sequence of amino acids of a polypeptide or a sequence of nucleotides in a nucleic acid molecule and includes deletions, insertions, and replacements of amino acids and nucleotides, respectively.

For purposes herein, amino acid replacements (or substitutions), deletions and/or insertions, can be made in any of the valencene synthases provided herein. Modifications can be made by making conservative amino acid replacements and also non-conservative amino acid substitutions. For example, amino acid replacements that desirably or advantageously alter properties of the valencene synthase can be made. For example, amino acid replacements can be made to the valencene synthase such that the resulting modified valencene synthase can produce more valencene from FPP compared to an unmodified valencene synthase.

As used herein, a peptide refers to a polypeptide that is from 2 to 40 amino acids in length.

As used herein, “primary sequence” refers to the sequence of amino acid residues in a polypeptide.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, the terms “homology” and “identity”” are used are used to describe relatedness between and among polypeptides (or encoding nucleic acid molecules). Identity refers to identical sequences; homology can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology varies). Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85: 2444 (other programs include the GCG program package (Devereux, J., et al., (1984) Nucleic Acids Research 12(I): 387), BLASTP, BLASTN, FASTA (Atschul, S. F., et al., J. Molec. Biol. 215:403 (1990); Guide to Huge Computers, Martin J. Bishop, ed., Academic Press, San Diego (1994), and Carillo et al. (1988) SIAM J Applied Math 48: 1073). For example, the BLAST function of the National Center for Biotechnology Information database can be used to determine identity. Other commercially or publicly available programs include DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48: 443, as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. (1986) Nucl. Acids Res. 14: 6745, as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polypeptide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, it also is understood that the terms “substantially identical” or “similar” varies with the context as understood by those skilled in the relevant art, but that those of skill can assess such.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, isolated or purified polypeptide or protein or biologically-active portion thereof is substantially free of cellular material or other contaminating proteins from the cell of tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as proteolytic and biological activities, of the substance. Methods for purification of the compounds to produce substantially chemically pure compounds are known to those of skill in the art. A substantially chemically pure compound, however, can be a mixture of stereoisomers. In such instances, further purification can increase the specific activity of the compound.

As used herein, “animal” is used in the broadest sense as used in the art, and encompasses vertebrates and invertebrates. Animals include, but are not limited to, amphibia, ayes, mammalian and reptilia. An animal can be a vertebrate, such as a mammal, avian or fish. An animal can be a human or a bovine, canine, caprine, cervine, cricetine, feline, galline, equine, lapine, murine, musteline and ovine.

An animal can be a human or other mammalian animals include primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), antelopes, buffalos, camels, deer, donkeys, rabbits, and rodents (e.g., guinea pigs, squirrels, rats, mice, gerbils, and hamsters).

As used herein, “companion animal” refers to an animal kept as a pet for companionship. Companion animals often are dogs, cats or horses, but also can include hamsters, gerbils, rabbits, guinea pigs, rats, mice, pot bellied pigs and pet birds.

As used herein, an acyclic pyrophosphate terpene precursor is any acyclic pyrophosphate compound that is a precursor to the production of at least one terpene, including, but not limited, farnesyl-pyrophosphate (FPP), to geranyl-pyrophosphate (GPP), and geranylgeranyl-pyrophosphate (GGPP). Acyclic pyrophosphate terpene precursor are thus substrates for terpene synthases.

As used herein, a terpene is an unsaturated hydrocarbon based on the isoprene unit (C₅H₈), and having a general formula C₁₀H₆. Reference to a terpene includes acyclic, monocyclic and polycyclic terpenes. Terpenes include, but are not limited to, monoterpenes, which contain 10 carbon atoms; sesquiterpenes, which contain 15 carbon atoms; diterpenes, which contain 20 carbon atoms, and triterpenes, which contain 30 carbon atoms. Reference to a terpene also includes stereoisomers of the terpene.

As used herein, a terpene synthase is a polypeptide capable of catalyzing the formation of one or more terpenes from an acyclic pyrophosphate terpene precursor, for example, FPP, GPP or GGPP.

As used herein, valencene is a sesquiterpene having the following structure:

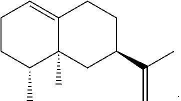

Reference to valencene includes reference to any isomer thereof, including, but not limited to (+)-valencene.

As used herein, a “valencene synthase” or “valencene synthase polypeptide” is a polypeptide capable of catalyzing the formation of valencene from an acyclic pyrophosphate terpene precursor, typically farnesyl diphosphate (FPP). Any valencene synthase that can be used for production of valencene in vivo or in vitro is contemplated. Included among these is any valencene synthase that has greater than 62%, 63%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity with the valence synthase set forth in SEQ ID NO:2. Valencene can be the only product or one of a mixture of products formed from the reaction of an acyclic pyrophosphate terpene precursor with a valencene synthase. The amount of valencene produced from the reaction of a valencene synthase with an acyclic pyrophosphate terpene precursor typically is at least or about 1%, 5%, 10%, 20%, 30%, 40%, 50%, 60%, 70%, 80%, 90% or more of the total amount of terpene produced in the reaction. In some instances, valencene is the predominant terpene produced (i.e. present in greater amounts than any other single terpene produced from the reaction of an acyclic pyrophosphate terpene precursor with a valencene synthase).

Reference to a valencene synthase includes any valencene synthase polypeptide including, but not limited to, a recombinantly produced polypeptide, a synthetically produced polypeptide and a valencene synthase polypeptide extracted or isolated from cells and plant matter including, but not limited to, citrus peel. Exemplary valencene synthase polypeptides include those isolated from citrus fruit, grapevine flowers (e.g. Vitis vinifera L. cv. Gewürztraminer and Vitis vinifera L. cv. Cabernet Sauvignon (see, Lucker et al., (2004) Phytochemistry 65(19):2649-59 and Martin et al., (2009) Proc. Natl. Acad. Sci, USA 106:7245-7250) and perilla (green shiso). Exemplary of valencene synthases are Citrus valencene synthase (CVS), including but not limited to, valencene synthase from Citrus sinensis (Sweet orange) (SEQ ID NOS:2 and 3) and Citrus x paradisi (Grapefruit) (SEQ ID NOS:4, 5 and 9).

Reference to valencene synthase includes valencene synthase from any genus or species, and included allelic or species variants, variants encoded by splice variants, and other variants thereof, including polypeptides that have at least 40%, 45%, 50%, 55%, 65%, 70%, 75%, 80%, 85%, 90%, 95%, 96%, 97%, 98%, 99% or more sequence identity to the valencene synthase set forth in SEQ ID NO:2. Valencene synthase also includes fragments thereof that retain valencene synthase activity.

As used herein, “valencene synthase activity” refers to the ability to catalyze the formation of valencene from an acyclic pyrophosphate terpene precursor, such as farnesyl diphosphate (FPP). Methods to assess valencene formation from the reaction of a synthase with an acyclic pyrophosphate terpene precursor, such as FPP, are well known in the art and described herein. For example, the synthase can be expressed in a host cell, such as a yeast cell, that also produces FPP. The production of valencene can then be assessed and quantified using, for example, gas chromatography-mass spectrometry (GC-MS) (see Examples below). Generally, a synthase can be considered to exhibit valencene synthase activity or the ability to catalyze the formation of valencene from an acyclic pyrophosphate terpene precursor such as FPP if the amount of valencene produced from the reaction is at least or about 1%, 5%, 10%, 20%, 30%, 40%, 50%, 60%, 70%, 80%, 90% or more of the total amount of terpene produced in the reaction.

As used herein, “wild-type” with reference to valencene synthase refers to a valencene synthase polypeptide encoded by a native or naturally occurring valencene synthase gene, including allelic variants, that is present in an organism, including a plant, in nature. Reference to wild-type valencene synthase without reference to a species is intended to encompass any species of a wild-type valencene synthase. The amino acid sequence of exemplary valencene synthases are set forth in SEQ ID NO: 2, (isolated from Citrus sinensis cv. Valencia, Citrus sinensis cv. Cara Cara and Citrus paradisi), SEQ ID NO:3 (isolated from Citrus sinensis cv. Valencia); and SEQ ID NO:4 (isolated from Citrus x paradisi) and SEQ ID NO:5 (isolated from Citrus x paradisi).

As used herein, species variants refer to variants in polypeptides among different species, including different citrus species, such Citrus sinensis and Citrus x paradisi.

As used herein, allelic variants refer to variations in proteins among members of the same species.

As used herein, a splice variant refers to a variant produced by differential processing of a primary transcript of genomic DNA that results in more than one type of mRNA.

As used herein, “modified valencene synthase polypeptide” refers to a valencene synthase polypeptide that has one or more amino acid differences compared to an unmodified or wild-type valencene synthase polypeptide. The one or more amino acid differences can be amino acid mutations such as one or more amino acid replacements (substitutions), insertions or deletions, or can be insertions or deletions of entire domains, and any combinations thereof. Typically, a modified valencene synthase polypeptide has one or more modifications in primary sequence compared to an unmodified or wild-type valencene synthase polypeptide. For example, a modified valencene synthase polypeptide provided herein can have 1, 5, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 105, 110, 115, 120, 125, 130, 135 or more amino acid differences compared to an unmodified valencene synthase polypeptide. Any modification is contemplated as long as the resulting polypeptide exhibits at least one valencene synthase activity associated with a wild-type valencene synthase polypeptide, such as, for example, catalytic activity, the ability to bind FPP, and/or the ability to catalyze the formation of valencene from FPP.

As used herein, reference to a modified valencene synthase polypeptide producing valencene from FPP in an amount that is greater than the amount of valencene produced from FPP by a reference valencene synthase, such as a wild-type valencene synthase, indicates that the modified valencene synthase produces at least or about 10% more valencene from FPP than the reference valencene synthase produces. For example, such a modified valencene synthase polypeptide can produce 10%, 11%, 12%, 13%, 14%, 15%, 20%, 30%, 40%, 50%, 60%, 70%, 80%, 90%, 100%, 110%, 120%, 130%, 140%, 150%, 160%, 170%, 180%, 200%, 250%, 300%, 350%, 400%, 500%, 600%, 700%, 800%, 900%, 1000%, 2000%, 5000% or more valencene from FPP compared to the amount of valencene produced from FPP by a reference valencene synthase. The amount of valencene produced from FPP by a valencene synthase can be assessed by any method known in the art. When comparing the amount of valencene produced from FPP by two valencene synthases, such as a modified valencene synthase and a reference valencene synthase, such as a wild-type valencene synthase, it is understood that the assay is performed under the same conditions for each synthase. In one example, the amount of valencene produced from FPP by two valencene synthases, such as a modified valencene synthase and a reference valencene synthase, is assessed by expressing the modified valencene synthase and the reference valencene synthase separately in a yeast cell of the same strain (wherein expression is from the same expression vector) that also produces FPP, and culturing the cells under the same conditions such that valencene is produced. The amount of valencene produced in the cell culture expressing the modified valencene synthase is compared to the amount of valencene produced in the cell culture expressing the reference valencene synthase, using methods of quantification well known in the art, such as GC-MS.

As used herein, corresponding residues refers to residues that occur at aligned loci. Related or variant polypeptides are aligned by any method known to those of skill in the art. Such methods typically maximize matches, and include methods such as using manual alignments and by using the numerous alignment programs available (for example, BLASTP) and others known to those of skill in the art. By aligning the sequences of polypeptides, one skilled in the art can identify corresponding residues, using conserved and identical amino acid residues as guides. For example, by aligning the sequences of valencene synthase polypeptides, one of skill in the art can identify corresponding residues, using conserved and identical amino acid residues as guides. For example, the tyrosine in amino acid position 221 (Y221) of SEQ ID NO:2 corresponds to the cysteine in amino acid position 221 (C221) of SEQ ID NO:3. In other instances, corresponding regions can be identified. For example, the unstructured loop 2 of valencene synthase (amino acids 53-58 of SEQ ID NO:2) corresponds to amino acids 58-63 of the tobacco epi-aristolochene synthase (TEAS). One skilled in the art also can employ conserved amino acid residues as guides to find corresponding amino acid residues between and among terpene synthases. For example, amino acid residues R264, W273, T403, Y404, C441 and D445 of the valencene synthase set forth in SEQ ID NO:2 correspond to amino acid residues R264, W273, T403, Y404, C440 and D444 of the tobacco epi-aristolochene synthase. Corresponding positions also can be based on structural alignments, for example by using computer simulated alignments of protein structure. In other instances, corresponding regions can be identified. Thus, reference to a modification, such as an amino acid replacement, that corresponds to, for example, Y221V in SEQ ID NO:2, includes amino acid replacement of the tyrosine at position 221 of SEQ ID NO:2 with a valine; and also includes replacement of the endogenous amino acid residue at the position corresponding to position 221 of SEQ ID NO:2 in any other similar or related polypeptide, with a tyrosine. For example, also included would be replacement of the cysteine at position 221 of SEQ ID NO:3 with a tyrosine (C221V).

As used herein, domain or region (typically a sequence of three or more, generally 5 or 7 or more amino acids) refers to a portion of a molecule, such as a protein or the encoding nucleic acids, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as catalytic activity. A protein can have one, or more than one, distinct domains. For example, a domain can be identified, defined or distinguished by homology of the sequence therein to related family members, such as other terpene synthases. In another example, a domain can be distinguished by its function, such as by catalytic activity, or an ability to interact with a biomolecule, such as substrate binding or metal binding. In some examples, a domain independently can exhibit a biological function or property such that the domain independently or fused to another molecule can perform an activity, such as, for example catalytic activity or substrate binding. A domain can be a linear sequence of amino acids or a non-linear sequence of amino acids. Many polypeptides contain a plurality of domains. Such domains are known, and can be identified by, those of skill in the art. For exemplification herein, definitions are provided, but it is understood that it is well within the skill in the art to recognize particular domains by name. If needed appropriate software can be employed to identify domains. For example, as discussed above, corresponding domains in different terpene synthases can be identified by sequence alignments, such as using tools and algorithms well known in the art (for example, BLASTP).

As used herein, the phrase “a property of the modified terpene synthase is improved compared to the first terpene synthase” refers to a desirable change in a property of a modified terpene synthase compared to a terpene synthase that does not contain the modification(s). Typically, the property or properties are improved such that the amount of a desired terpene produced from the reaction of a substrate with the modified terpene synthase is increased compared to the amount of the desired terpene produced from the reaction of a substrate with a terpene synthase that is no so modified. Exemplary properties that can be improved in a modified terpene synthase include, for example, terpene yield, catalytic activity, catalytic activity, product distribution; substrate specificity; regioselectivity and stereoselectivity. One or more of the properties can be assessed using methods well known in the art to determine whether the property had been improved (i.e. has been altered to be more desirable for the production of a desired terpene or terpenes).

As used herein, terpene yield refers to the amount (in weight or weight/volume) of terpene produced from the reaction of an acyclic pyrophosphate terpene precursor with a terpene synthase. Reference to total terpene yield refers to the total amount of all terpenes produced from the reaction, while reference to specific terpene yield refers to the amount of a specific terpene (e.g. valencene), produced from the reaction.

As used herein, an improved terpene yield refers to an increase in the total amount of terpene (i.e. improved total terpene yield) or an increase in the specific amount of terpene (i.e. improved specific terpene yield) produced from the reaction of an acyclic pyrophosphate terpene precursor with a modified terpene synthase compared to the amount produced from the reaction of the same acyclic pyrophosphate terpene precursor with a terpene synthase that is not so modified. The amount of terpene (total or specific) produced from the reaction of an acyclic pyrophosphate terpene precursor with a modified terpene synthase can be increased by at least or about 5%, 10%, 15%, 20%, 30%, 40%, 50%, 60%, 70%, 80%, 90%, 100% or more compared to the amount of terpene produced from the reaction of the same acyclic pyrophosphate terpene precursor under the same conditions with a terpene synthase that is not so modified.

As used herein, substrate specificity refers to the preference of a valencene synthase for one target substrate over another, such as one acyclic pyrophosphate terpene precursor (e.g. farnesyl-pyrophosphate (FPP), geranyl-pyrophosphate (GPP), or geranylgeranyl-pyrophosphate (GGPP) over another. Substrate specificity can be assessed using methods well known in the art, such as those that calculate k_{cat}/Kₘ.

As used herein, altered specificity refers to a change in substrate specificity of a modified terpene synthase polypeptide (such as a modified valencene synthase polypeptide) compared to a terpene synthase that is not so modified (such as, for example, a wild-type valencene synthase). The specificity (e.g. k_{cat}/Kₘ) of a modified terpene synthase polypeptide for a substrate, such as FPP, GPP or GGPP, can be altered by at least or about 10%, 15%, 20%, 30%, 40%, 50%, 60%, 70%, 80%, 90%, 100% or more compared to the specificity of a starting valencene synthase for the same substrate.

As used herein, improved substrate specificity refers to a change or alteration in the substrate specificity to a more desired specificity. For example, an improved substrate specificity can include an increase in substrate specificity of a modified terpene synthase polypeptide for a desired substrate, such as FPP, GPP or GGPP. The specificity (e.g. k_{cat}/Kₘ) of a modified terpene synthase polypeptide for a substrate, such as FPP, GPP or GGPP, can be increased by at least or about 10%, 15%, 20%, 30%, 40%, 50%, 60%, 70%, 80%, 90%, 100% or more compared to the specificity of a terpene synthase that is not so modified.

As used herein, “product distribution” refers to the relative amounts of different terpenes produced from the reaction between an acyclic pyrophosphate terpene precursor, such as FPP, and a terpene synthase, including the modified valencene synthase polypeptides provided herein. For example, the product distribution resulting from reaction of FPP with a valencene synthase can be 90% (weight/volume) valencene and 10% (weight/volume) germacrene A. Methods for assessing the type and amount of a terpene in a solution are well known in the art and described herein, and include, for example, gas chromatography-mass spectrometry (GC-MS) (see Examples below).

As used herein, an altered product distribution refers to a change in the relative amount of individual terpenes produced from the reaction between an acyclic pyrophosphate terpene precursor, such as FPP, and a terpene synthase, such as valencene synthase. Typically, the change is assessed by determining the relative amount of individual terpenes produced from the acyclic pyrophosphate terpene precursor using a first synthase (e.g. wild-type synthase) and then comparing it to the relative amount of individual terpenes produced using a second synthase (e.g. a modified synthase). An altered product distribution is considered to occur if the relative amount of any one or more terpenes is increased or decreased by at least or by about 5%, 10%, 15%, 20%, 25%, 30%, 35%, 40%, 50%, 60%, 70%, 80 or more.

As used herein, an improved product distribution refers to a change in the production distribution to one that is more desirable, i.e. contains more desirable relative amounts of terpenes. For example, an improved product distribution can contain an increased amount of a desired terpene and a decreased amount of a terpene that is not so desired. The amount of desired terpene in an improved production distribution can be increased by at least or by about 5%, 10%, 15%, 20%, 25%, 30%, 35%, 40%, 50%, 60%, 70%, 80% or more. The amount of a terpene that is not desired in an improved production distribution can be decreased by at least or by about 5%, 10%, 15%, 20%, 25%, 30%, 35%, 40%, 50%, 60%, 70%, 80% or more.

The term “substantially free of cellular material” includes preparations of valencene synthase or terpene products in which the valencene synthase or terpene is separated from cellular components of the cells from which it is isolated or produced. For example, the term “substantially free of cellular material” includes preparations of valencene synthase or terpene products having less that about 30%, 20%, 10%, 5% or less (by dry weight) of non-valencene synthase or terpene proteins or products, including cell culture medium.

As used herein, production by recombinant methods by using recombinant DNA methods refers to the use of the well known methods of molecular biology for expressing proteins encoded by cloned DNA.

As used herein, vector (or plasmid) refers to discrete elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as bacterial artificial chromosomes, yeast artificial chromosomes and mammalian artificial chromosomes. Selection and use of such vehicles are well known to those of skill in the art.

As used herein, expression refers to the process by which nucleic acid is transcribed into mRNA and translated into peptides, polypeptides, or proteins. If the nucleic acid is derived from genomic DNA, expression can, if an appropriate eukaryotic host cell or organism is selected, include processing, such as splicing of the mRNA.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer and a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, an adenovirus refers to any of a group of DNA-containing viruses that cause conjunctivitis and upper respiratory tract infections in humans.

As used herein, naked DNA refers to histone-free DNA that can be used for vaccines and gene therapy. Naked DNA is the genetic material that is passed from cell to cell during a gene transfer processed called transformation or transfection. In transformation or transfection, purified or naked DNA that is taken up by the recipient cell will give the recipient cell a new characteristic or phenotype.

As used herein, operably or operatively linked when referring to DNA segments means that the segments are arranged so that they function in concert for their intended purposes, e.g., transcription initiates in the promoter and proceeds through the coding segment to the terminator.

As used herein, a “chimeric protein” or “fusion protein” refers to a polypeptide operatively-linked to a different polypeptide. A chimeric or fusion protein provided herein can include one or more valencene synthase polypeptides, or a portion thereof, and one or more other polypeptides for any one or more of a transcriptional/translational control signals, signal sequences, a tag for localization, a tag for purification, part of a domain of an immunoglobulin G, and/or a targeting agent. A chimeric valencene synthase polypeptide also includes those having their endogenous domains or regions of the polypeptide exchanged with another polypeptide. These chimeric or fusion proteins include those produced by recombinant means as fusion proteins, those produced by chemical means, such as by chemical coupling, through, for example, coupling to sulfhydryl groups, and those produced by any other method whereby at least one polypeptide (i.e. valencene synthase), or a portion thereof, is linked, directly or indirectly via linker(s) to another polypeptide.

As used herein, recitation that a polypeptide “consists essentially” of a recited sequence of amino acids means that only the recited portion, or a fragment thereof, of the full-length polypeptide is present. The polypeptide can optionally, and generally will, include additional amino acids from another source or can be inserted into another polypeptide

As used herein, the singular forms “a,” “an” and “the” include plural referents unless the context clearly dictates otherwise. Thus, for example, reference to polypeptide, comprising “an amino acid replacement” includes polypeptides with one or a plurality of amino acid replacements.

As used herein, ranges and amounts can be expressed as “about” a particular value or range. About also includes the exact amount. Hence “about 5%” means “about 5%” and also “5%.”

As used herein, “optional” or “optionally” means that the subsequently described event or circumstance does or does not occur, and that the description includes instances where said event or circumstance occurs and instances where it does not. For example, an optionally step of isolating valencene means that the valencene is isolated or is not isolated.

As used herein, “control” when used in the context to “control an insect or pest” means to kill, repel, expel, incapacitate, deter, eliminate, alleviate, reduce in number and/or eradicate.

As used herein, “knockdown” refers to the inability of the insect or pest to move toward heat, indicating that that insect or pest is sickly, but not necessarily dying.

As used herein, whenever a numerical range, such as 1-10 or 5% to 50%, appears herein, the range encompasses the entire range bounded by the first and last recited value. For example, “an alkyl of 1 to 20 carbon atoms” means that an alkyl group can contain only 1 carbon atom, 2 carbon atoms, 3 carbon atoms, etc., up to and including 20 carbon atoms. Another example includes “a formulation including 1% to 10% by weight oil,” which means that the formulation includes by weight 1%, 1.1%, 1.2%, 1.3%, 1.4%, 1.5%, 1.6%, 1.7%, 1.8%, 1.9%, 2%, 2.1% . . . , 9.7%, 9.8%, 9.9% or 10% oil.

As used herein, “insecticide” refers to a substance used to prevent, destroy, repel, mitigate, or kill insects.

As used herein, “organophosphate” refers to an organophosphorous compound that displays anti-cholinesterase activity.

As used herein, “malathion” refers to an organophosphate pesticide that has the chemical name S-(1,2-dicarbethoxyethyl)-O,O-dimethyldithiophosphate or ((dimethoxy-phosphinothioyl)thio)butanedioic acid, diethyl ester (CAS No. 121-75-5). U.S. Pat. Nos. 3,352,664, 3,396,223, and 3,515,782 describe the use of malathion in pesticides. The disclosure of these references is incorporated by reference.

As used herein, “mammal” refers to a class of higher vertebrates comprising man and all other animals that nourish their young with milk secreted by mammary glands and that have skin that is more or less covered with hair. Exemplary mammals include humans, monkeys, rodents, sheep, goats, pigs, dairy and beef cattle, dogs, cats, horses, rabbits, guinea pigs and ferrets.

As used herein, “pests” refers to insects that are detrimental, annoying or a nuisance to plants or animals, including humans or domesticated animals. Exemplary pests include ants, ants, bedbugs, carpet beetles, centipedes, chiggers, drain flies, dust mites, earwigs, fleas, flies, gnats, hornets, lice, millipedes, mites, mosquitoes, roaches scabies, silverfish, spiders, stinkbugs, termites, ticks, wasps, weevils and yellow jackets.

As used herein, “bedbug” refers to one of the parasitic Heteroptera:Cimicidae insects, including the common bedbug (Cimex lectularius), tropical bedbug (Cimex hemipterus), Leptocimex boueti, which infests bats and humans, Cimex pilosellus, Cimex pipistrella, and Haematosiphon inodora.

As used herein, “lice” refers to insects of the order Phthiraptera, which is sometimes split into the order Anoplura, the sucking lice, and the order Mallophaga, the biting lice. All Phthiraptera are wingless external parasites of birds and mammals.

As used herein, the term “subject” is an animal, typically a mammal or bird. Included are humans, primates, cattle, pigs, rabbits, goats, sheep, mice, rats, guinea pigs, hamsters, cats, dogs, horses, chickens, ducks, turkeys and others.

As used herein, “sufficient to kill” refers to an amount of an agent that is sufficient to kill an organism, such as an ectoparasite or pest.

As used herein, the term “carrier” refers to a diluent, adjuvant, excipient, auxiliary agent or vehicle with which and active agent can be delivered. A carrier can be used in formulations to facilitate mixing, storage, transport and/or handling of an ingredient.

As used herein, “grapefruit oil” refers to the essential oil expressed from the rinds of the large fruits from cultivated grapefruit trees (Citrus paradisi Macfad., or Citrus x paradisi). The oil is usually extracted by cold compress of the peel of the fruit. The aroma is characteristic and regarded as a fresh, sweet citrus. The chemical constituents include various amounts of nootkatone, p-menth-1-en-8-thiol, ethyl butyrate, (Z)-3-hexenal, 1-hepten-3-one, 4-mercapto-4-methyl-2-pentanone, d-limonene, decanal and furano-coumarin (e.g., see Ericson et al., Drug Metab Dispos 25(11): 1228-1233 (1997).

As used herein, “residual action” refers to the length of time a compound or composition exists in a particular environment and retains activity sufficient to be effective for its intended purpose.

As used herein, “treatment” means any manner in which one or more of the symptoms of a disease or disorder are ameliorated or otherwise beneficially altered. Treatment also encompasses any use of the compositions herein, such as use for treating, repelling and/or eradicating any ectoparasite or pest. Prevent means to reduce the risk of getting a disease or disorder.

As used herein, “amelioration” of the symptoms of a particular disorder by administration of a particular composition refers to any lessening, whether permanent or temporary, lasting or transient that can be attributed to or associated with administration of the composition.

As used herein, the term “contacting” refers to bringing two or more materials into close enough proximity that they can interact. Contacting can be accomplished in a vessel such as a test tube or a Petri dish, or in a room or barn, or in a field.

As used herein, a composition refers to any mixture of two or more ingredients. It can be a solution, a suspension, a liquid, a powder, a paste, aqueous, non-aqueous or any combination thereof.

As used herein, a combination refers to any association between two or more items.

As used herein, fluid refers to any composition that can flow. Fluids thus encompass compositions that are in the form of liquids, semi-solids, pastes, solutions, aqueous mixtures, gels, lotions, creams and other such compositions.

Unless otherwise indicated, as expressed in the present specification as well as in the set of claims, % (percentage) refers to weight % (percent by weight).

As used herein, “repel” when used in the context of “repelling an insect” means to repulse, ward off, drive back or keep away from a treated surface, such that at any given time, there are fewer insects or pests on a treated locus then on an untreated locus under the same conditions. Although an insect or pest can land on or cross over a treated surface, the insect does not stay on the treated surface for a prolonged period of time or does not stay to probe or bite or otherwise damage the surface.

As used herein, the term “insect repellent” refers to a compound or composition conferring on a subject or locus protection from insects or pests when compared to no treatment at all.

As used herein, “protection” refers to a reduction in numbers of insects, and can, e.g., be usefully determined by measuring mean complete protection time (“mean CPT”) in tests in which insect behavior toward treated animals, including humans, and treated inanimate surfaces is observed.

As used herein, “mean CPT” refers to the mean length of time before the first landing, probing or biting (in the case of a biting insect) or crawling (in the case of a crawling insect such as a tick or chigger) on a treated surface is observed over two or more repetitions of tests. This can be measured using any method or technique known in the art (e.g., see Fradin et al. New England Journal of Medicine 347: 13-18 (2002)).

As used herein, the term “detergency builder” refers to an agent that serves to enhance the cleaning capacity or cleansing action of detergent compounds in a cleaning composition. A detergency builder has the property of improving detergency levels in detergent compositions and permit the attainment of cleaning performance that is superior to compositions that do not include a detergency builder.

As used herein, “pest” refers to any organism whose existence it can be desirable to control. Pests can include, for example, arthropod species, such as, for example, an insect, an arachnid, or an arachnoid, bugs, flies and parasites. The pest can be a species belonging to an animal order, such as, for example, Acari, Anoplura, Araneae, Blattodea, Coleoptera, Collembola, Diptera, Grylloptera, Heteroptera, Homoptera, Hemiptera:Cimicidae, Hymenoptera, Isopoda, Isoptera, Lepidoptera, Mantodea, Mallophaga, Neuroptera, Odonata, Orthoptera, Psocoptera, Siphonaptera, Symphyla, Thysanura, Thysanoptera,

As used herein, “pest control” refers to a disruption in a target pest's status that can result in repellency, knockdown, or killing of the target pest.

As used herein, a “derivative” refers to chemical substance derived from another substance either directly or by modification or partial substitution, and can include differences in one atom, element or group or more than one atom, element or group.

As used herein, an “analog” with respect to chemical compounds refers to a chemical compound that has a similar structure and similar chemical properties to those of another compound, but differs from it by a single atom, element or group.

As used herein, “finely divided” refers to a powder in which the majority (>50%) of the particles therein have a particle size which is less than about 200 μm in diameter.

As used herein, a “granule” refers to porous or nonporous particles (such as crushed rock or stone) as well as agglomerated smaller particles, for example, agglomerated powder particles, that are relatively large, with a particle size which is about 200-2500 microns in diameter typically.

As used herein a “volatile silicone” refers to those silicone materials that have a measurable vapor pressure at 25° C. Such vapor pressures can be in the range from about 0.01 mmHg to about 6 mmHg. Conversely, the term “non-volatile silicone” refers to those silicone materials that do not have a measurable vapor pressure at 25° C.

As used herein, a “dispersing agent” refers to a compound that can promote uniform separation of particles. Typical dispersing agents are surface active agents.

As used herein, a “surface-active agent” refers to any compound that reduces surface tension when dissolved in water or water solutions, or that reduces interfacial tension between two liquids, or between a liquid and a solid. Examples of surfactants include, but are not limited to: (1) fatty acid esters such as glycerol esters, PEG esters, and sorbitan esters, including ethylene glycol distearate, ethylene glycol monostrearate, glycerol mono and/or dioleate, PEG dioleate, PEG monolaurate, sorbitan monolaurate, sorbitan trioleate; (2) nonionic ethoxylates such as alkylphenol ethoxylates, alcohol ethoxylates, alkylamine ethoxylates, such as octylphenol ethoxylate, nonylphenol ethoxylate, alkylamine ethoxylates; (3) nonionic surfactants such as 2,4,7,9-tetramethyl-5-decyn-4,7-diol; and (4) ethylene oxide/propylene oxide copolymers.

As used herein, “viscosity” refers to a physical property of a fluid that determines the internal resistance to shear forces; viscosity can be expressed in centipoise (cP).

As used herein, a “gelling agent” refers to a compound that facilitates the gelation of a liquid.

As used herein, a “softening active agent” refers to any compound used to soften clothes when washed or dried. One class of softening active agents includes the quaternary amines, or “quats” or “quaternaries.”. Exemplary quaternary amines include the monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries.

As used herein, “moribund” refers to a pest or insect that is unable to move towards heat twenty-four hours after treatment; the louse is dead or dying.

### B. NOOTKATONE

The compositions provided herein include nootkatone (4,4a,5,6,7,8-hexahydro-6-isopropenyl-4,4a-dimethyl-2(3H)-naphtalenone), or a derivative or analog of nootkatone or other related compound. Nootkatone has the following structure:

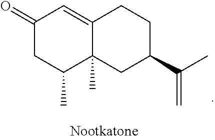 [SMILES: C=C(C)[C@@H]1CCC2=CC(=O)C[C@@H](C)[C@]2(C)C1]

Nootkatone is a sesquiterpene naturally found in citrus oils, such as orange and grapefruit, and other plant matter. In particular, nootkatone is an aromatic of grapefruit. Nootkatone is considered to have excellent organoleptic qualities and in particular to impart a typical grapefruit taste and smell. Nootkatone has been used in the flavor and fragrance industry, such as a component in perfumes and as a flavor in soft drinks and other beverages.

Analogs and derivates of nootkatone are known in the art. These include, but are not limited to, nootkatone-11,12-epoxide, nootkatone-1,10-epoxide, nootkatone-1,10-11,12-diepoxide, tetrahydronootkatone, 1,10-dihydronootkatone, isonootkatone and nootkatol (see U.S. Pat. Nos. 6,897,244 and 7,112,700 and WO 2002/050053). For example, conversion of an enone to the corresponding saturated ketone can be carried out with an alkali metal (e.g., Na or Li) in the presence of a proton source (such as liquid ammonia, ethanol, or both) to produce tetrahydronootkatone (e.g., see Adcock et al., J. Org. Chem. 47: 2951 (1982)). Nootkatone also be converted into isonootkatone, tetrahydronootkatone, 11,12-dihyydronootkatone or 1,10-dihydronootkatone using well known methods (Stevens et al., J. Sci. Fd. Agric. 21: 590-593 (1970)). Zhu et al. describes the conversion of nootkatone into nootkatol (J. Chem. Ecol. 29: 2695-2701 (2003)).

### C. METHODS OF MAKING OR OBTAINING NOOTKATONE

Nootkatone, which is the dominant grapefruit aroma, is an oxidized product of valencene. Nootkatone and its derivatives and analogs can be obtained commercially, can be extracted and/or isolated from cells or can be made by biosynthetic, chemical, recombinant and/or enzymatic methods. For example, commercial preparations of nootkatone include, but are not limited to, nootkatone purchased from Sigma-Aldrich (Product No. 74437 or Product No. 93785; St. Louis, Mo.); Aromor Flavors & Fragrances Ltd. (Code No. 4101; Israel); and Bedoukian Research, Inc. (Product No. 800; Danbury, Conn.).

Nootkatone also can be extracted and/or isolated from cells. For example, Nootkatone can be isolated from natural sources such as Valencia oranges or grapefruit, particularly grapefruit oil, using generally known methods for isolating and purifying terpenes. Methods of extracting or making nootkatone are well known in the art. Exemplary of such methods are described in U.S. Pat. No. 4,693,905; U.S. Pat. No. 4,973,485; U.S. Pat. No. 5,260,086; U.S. Pat. No. 6,495,193; and U.S. Patent Application No. US20030185956 and US20030203090.

Nootkatone is formed by the oxidation of valencene. Valencene (1,2,3,5,6,7,8,8a-octahydro-7-isopropenyl-1,8a-dimethyl-naphthalene) can undergo regioselective hydroxylation to form 2-hydroxy valencene, which is further oxidized to form nootkatone. The selective oxidation of valencene at allylic C2-position yields cis and trans-nootkatol, which can be further oxidized to nootkatone. Thus, nootkatone also can be generated by oxidation from valencene using biosynthesis, chemical or bioconversion methods. Oxidation of valencene can be carried out through chemical or biosynthetic means (see e.g. U.S. Pat. No. 5,847,226, Eur. Pat. No. EP1083233; Girhard et al., (2009) Microb. Cell. Fact. 8:36; Fraatz et al., (2009) Appl Microbiol Biotechnol. 83(1):35-41; Furusawa et al. (2005) Chem Pharm. Bull. 53:1513-1514; Salvador et al., (2002) Green Chemistry, 4, 352-356;). Biochemical oxidation can be effected by a hydroxylase, or alternatively can be effected by sequential enzyme mediated reactions. In some examples, valencene is converted to nootkatone using chromium trioxide or a silica phosphonate-immobilized chromium (III) catalyst. The valencene that is used in such processes can be extracted or isolated from cells or can be recombinantly generated as described below in cells expressing valencene synthase. Nootkatone formation can be confirmed and/or quantified by any of the chromatographic techniques described herein.

For example, a large number of chemical and synthetic methods are known in the art for obtaining nootkatone by oxidation of valencene. Chemical synthesis of nootkatone from valencene can be effected with tert-butyl chromate (Hunter et al. (1965) J. Food Sci, 30:876); tert-butyl peracetate (Wilson et al. (1978) J. Agric. Food Chem., 26:1430); via copper(I)-mediated oxidation by alkyl hydroperoxides (Salvador et al. (1997) Tetrahedron Lett. 38:119-122) and with surface-functionalized silica and metal catalysts such as CO²⁺ and Mn²⁺ (Salvador et al. (2002) Green Chem., 4:352-356. Other chemical methods for the production of nootkatone are known (e.g., see Hunter et al., Conversion of valencene to nootkatone, J Food Sci 30: 876-878 (1965) and Pesaro et al., The total synthesis of racemic nootkatone, Chem Commun (London) 19: 1152-1154 (1968)).

Biological oxidation of valencene is another method of producing nootkatone. Biotechnological processes include cellular and cell-free processes. For example, cellular biological processes that can result in oxidation include, but are not limited to, the manufacturing of nootkatone with green algae like Chlorella or Euglena, or fungi (Furusawa et al. (2005) Chem. Pharm. Bull, 53:1423-1429). Furusawa et al. describes biotransformation of (+)-valencene obtained from Valencia oranges to nootkatone in high yield using species of the green algae Chlorella and fungi, such as Botryosphaeria dothidea, Botryodiplodia theobromae and species of Mucor. U.S. Pat. No. 7,214,507 describes a process for production of nootkatone by bioconversion using a chicory root extract transformed to contain a valencene synthase. The oxidation of nootkatone can be achieved by whole-cell oxidation, such as by using cells expressing a P450 enzyme. Several cytochrome P450 monooxygenases are known to one of skill in the art and are known to catalyze oxidation of nootkatone (see e.g. Girhard et al. (2009) Microbial Cell Factories, 8:36). Exemplary p450 enzymes include, but are not limited to, P450_{cam} from Pseudomonas putida and P450_{BM-3} from Bacillus megaterium, or modified forms thereof (see e.g. Sowden et al. (2005) Org. Biomol. Chem., 3:57-64) or CYP109B 1 from Bacillus subtilis (Girhard et al. (2009) Microbial Cell Factories, 8:36).

Cell free systems for biochemically producing nootkatone include conversion from valencene include using enzymes from Cichorium intybus L. roots (de Kraker et al. (2003) Tetrahedron, 59:409-418); lignin peroxidase and fungal laccase (see e.g. U.S. Pat. No. 6,200,786). For example, U.S. Pat. No. 6,200,786 describes a method of producing nootkatone by laccase catalyzed oxidation of valencene. The methods involves the conversion of valencene into valencene hydroperoxide, which is degraded to form nootkatone, and nootkatone then is recovered. Nootkatone also can be produced by oxidizing valencene to nootkatone (4,4a,5,6,7,8-hexahydro-6-isopropenyl-4,4a-dimethyl-2(3H)-naphtalenone), nootkatol (2,3,4,4a,5,6,7,8-octahydro-6-isopropenyl-4,4a-dimethyl-2-naphtalenol) or mixtures of nootkatone and nootkatol in the presence of an unsaturated fatty acid hydroperoxide (see e.g. U.S. Pat. No. 5,847,226).

Nootkatone also can be produced by first producing valencene using a cell strain, such as a S. cerevisiae or other yeast strain, containing a valencene synthase gene as described below. The valencene then can be oxidized, either biologically or chemically, to produce nootkatone.

### D. VALENCENE

Valencene and nootkatone are natural constituents of citrus oils, such as orange and grapefruit, and are widely used ingredients in perfumery and the flavor industry. Nootkatone can be produced from valencene. Valencene predominantly occurs in the essential oils or orange and grapefruit, but also has been identified in minor quantities in celery (Apium graveolens), mango (Mangifera indica), olives (Olea europea) and coral. Valencene can be extracted and isolated from oranges and grapefruits. For example, valencene can be obtained by fractionation of essential oils, for example by using extraction or distillation techniques. Essential oils are volatile oils, usually having the characteristic odor or flavor of the plant from which they are obtained, used to make perfumes and flavorings. Examples of essential oils include orange oil, bitter orange oil, grapefruit oil, lemon oil, tangerine oil, or citrus oil. Valencene also can be obtained from a commercial source (e.g. Valencene Pure™; Product Code No. 10.010.32 from Isobionics, Netherlands; and Valencene natural; Product No. W344303 from Sigma-Aldrich, St. Louis, Mo.). Valencene also can be produced from a cell strain expressing a valencene synthase gene and appropriate precursors.

1. Valencene Synthase

Valencene synthases are class 1 plant terpene cyclases, or terpene synthases, isoprenoid synthases or terpenoid cyclases, which convert farnesyl diphosphate into the sesquiterpene valencene and the valencene can then be converted to nootkatone by oxidation:

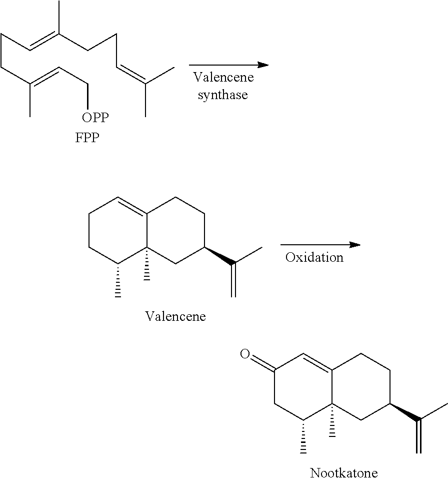

In the reaction scheme shown, farnesyl diphosphate is enzymatically converted into the sesquiterpene valencene (1,2,3,5,6,7,8,8a-octahydro-7-isopropenyl-1,8a-dimethyl-naphthalene), which then is converted by oxidation to nootkatone (4,4a,5,6,7,8-hexahydro-6-isopropenyl-4,4-a-dimethyl-2(3H)-naphtalenone) as described above using chemical or biological methods.

Valencene synthases have been isolated from a variety of sources, including citrus fruit, grapevine flowers and perilla (green shiso). In particular, citrus valencene synthase is a sesquiterpene synthase found in citrus fruit, such as oranges and grapefruit, which converts all-trans farnesyl diphosphate (FPP) into the sesquiterpene valencene. Several citrus valencene synthases have been identified and isolated to date. The amino acid sequences of the citrus valencene synthases are not necessarily species-specific, as synthases isolated from a particular species (e.g. Citrus sinensis) can have the same or different sequence from that of another synthase isolated from the same species, and can have the same or different sequence as a synthase isolated from a different species (e.g. Citrus paradisi). Citrus valencene synthases isolated and sequenced to date include the valencene synthase isolated from Citrus sinensis cv. Valencia (Valencia orange), which is a 548 amino acid polypeptide having an amino acid sequence set forth in SEQ ID NO:2 (encoded by the cDNA sequence set forth in SEQ ID NO:1). Citrus valencene synthase (CVS) has been identified in the flavedo (outer peel) of Citrus sinensis (Sweet orange) and Citrus x paradisi (Grapefruit) (see, Sharon-Asa et al., (2003) The Plant Journal 36:664-674; AF411120 and U.S. Pat. Nos. 7,273,735 and 7,442,785).

Valencene synthases also have been identified and isolated from grapevine flowers, including Vitis vinifera L. cv. Gewürztraminer and Vitis vinifera L. cv. Cabernet Sauvignon (see, Lucker et al., (2004) Phytochemistry 65(19):2649-59 and Martin et al., (2009) Proc. Natl. Acad. Sci. USA 106:7245-7250).

Valencene synthase polypeptides are described in the art. For example, see GenBank Accession Nos.: ACX70155.1; AAQ0468.1 (AF441124_{—}1); AAM0426.1, U.S. Pat. No. 7,273,735 and U.S. Pat. No. 7,790,426 (in particular, SEQ ID NO:4 and SEQ ID NO:5), International PCT Appl. No. PCT/IL2004/000795, which published as WO2005021705, International PCT Appl. No. PCT/NL2002/000591, which published as WO2003025193, and Chappell, (2004) Trends Plant Sci., 9: 266. The disclosure of each of these is incorporated by reference in its entirety. Also incorporated by reference is the disclosure of commonly owned U.S. Provisional Patent Application 61/455,990, filed Oct. 29, 2010, now U.S. Patent Publication No. 2012-0246767. Exemplary valencene synthase genes and encoding amino acids are set forth in any of SEQ ID NOS:1-10.

2. Production of Valencene

Valencene synthase polypeptides can be used to catalyze the formation of valencene from an acyclic pyrophosphate terpene precursor, such as FPP. Valencene synthases can be expressed in cells that co-express and/or overexpress FPP, such that valencene is produced by the pathway described above. In other examples, the valencene synthases can be expressed and purified from any suitable host cell. The purified synthases can then combined in vitro with a FPP to produce valencene.

In some examples, the modified valencene synthase provided herein is overexpressed and purified. The modified valencene synthase is then incubated with the substrate farnesyl diphosphate and valencene is produced. The pH of the solution containing FPP and valencene synthase can impact the amount of valencene produced (see e.g. U.S. Pat. Pub. No. 20100216186). An organic solvent is added to partition the valencene into the organic phase for analysis. Production of valencene and quantification of the amount of product are then determined using any method provided herein, such as gas chromatography (e.g. GC-MS) using an internal standard. Alternatively, the modified valencene synthase is expressed in host cells that also produce FPP, resulting in production of valencene. The valencene can then be extracted from the cell culture medium with an organic solvent and subsequently isolated and purified by any known method, such as column chromatography or HPLC, and the amount and purity of the recovered valencene are assessed. In some examples, the valencene is converted by oxidation to nootkatone either before or after purification.

Valencene can be produced by expressing a valencene synthase polypeptide in a cell line that produces FPP as part of the mevalonate-dependent isoprenoid biosynthetic pathway (e.g. fungi, including yeast cells and animal cells) or the mevalonate-independent isoprenoid biosynthetic pathway (e.g. bacteria and higher plants). In particular examples, valencene is produced by expressing a valencene synthase polypeptide in a cell line that has been modified to overexpress FPP. Exemplary of such cells are modified yeast cells. For example, yeast cells that have been modified to produce less squalene synthase or less active squalene synthase (e.g. erg9 mutants; see e.g. U.S. Pat. Nos. 6,531,303 and 6,689,593) are useful in the methods provided herein to produce valencene. Reduced squalene synthase activity results in accumulation of FPP in the host cell at higher levels compared to wild-type yeast cells, which in turn can result in increased yields of valencene production.

Exemplary modified yeast cells include, modified Saccharomyces cerevisiae strains CALI5-1 (ura3, leu2, his3, trp1, Aerg9::HIS3, HMG2cat/TRP1::rDNA, dpp1), and those described in U.S. Pat. Nos. 6,531,303 and 6,689,593 and published U.S. Patent Appl. No. US20040249219. Saccharomyces cerevisiae strain CALI5-1 is a derivative of SW23B#74 (described in U.S. Pat. Nos. 6,531,303 and 6,689,593, and Takahashi et al. (2007) (Biotechnol Bioeng. 97(1): 170-181), which itself is derived from wild-type strain ATCC 28383 (MATa). CALI5-1 was generated to have a decreased activity of the Dpp1 phosphatase (see e.g. U.S. Published Appl. No. US20040249219). Saccharomyces cerevisiae strain CALI5-1 contains, among other mutations, an erg9 mutation (the Aerg9::HIS3 allele) as well as an uncharacterized mutation supporting aerobic sterol uptake enhancement (sue). It also contains approximately 8 copies of the truncated HMG2 gene. The truncated form of HMG2 is driven by the GPD promoter and is therefore no longer under tight regulation, allowing for an increase in carbon flow to FPP. It also contains a deletion in the gene encoding diacylglycerol pyrophosphate (DGPP) phosphatase enzyme (dpp1), which limits dephosphorylation of FPP.

A host cell expressing a valencene synthase and farnesyl diphosphate, such as a host cell modified to overexpress farnesyl diphosphate, can be cultured using any suitable method well known in the art. A variety of fermentation methodologies can be utilized for the production of valencene from yeast cells expressing the modified valencene synthase polypeptides provided herein. For example, large scale production can be effected by either batch or continuous fermentation. A classical batch fermentation is a closed system where the composition of the medium is set at the beginning of the fermentation and not subject to artificial alterations during the fermentation. Thus, at the beginning of the fermentation the medium is inoculated with the desired microorganism or microorganisms and fermentation is permitted to occur without further addition of nutrients. Typically, the concentration of the carbon source in a batch fermentation is limited, and factors such as pH and oxygen concentration are controlled. In batch systems the metabolite and biomass compositions of the system change constantly up to the time the fermentation is stopped. Within batch cultures cells typically modulate through a static lag phase to a high growth log phase and finally to a stationary phase where growth rate is diminished or halted. If untreated, cells in the stationary phase will eventually die.

A variation on the standard batch system is the Fed-Batch system, which is similar to a typical batch system with the exception that nutrients are added as the fermentation progresses. Fed-Batch systems are useful when catabolite repression tends to inhibit the metabolism of the cells and where it is desirable to have limited amounts of substrate in the medium. Also, the ability to feed nutrients will often result in higher cell densities in Fed-Batch fermentation processes compared to Batch fermentation processes. Factors such as pH, dissolved oxygen, nutrient concentrations, and the partial pressure of waste gases such as CO are generally measured and controlled in Fed-Batch fermentations.

Production of the valencene also can be accomplished with continuous fermentation. Continuous fermentation is an open system where a defined fermentation medium is added continuously to a bioreactor and an equal amount of conditioned medium is removed simultaneously for processing. This system generally maintains the cultures at a constant high density where cells are primarily in their log phase of growth. Continuous fermentation allows for modulation of any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by the medium turbidity, is kept constant. Continuous systems aim to maintain steady state growth conditions and thus the cell loss due to the medium removal must be balanced against the cell growth rate in the fermentation. Methods of modulating nutrients and growth factors for continuous fermentation processes as well as techniques for maximizing the rate of product formation are well known in the art. Following cell culture, the cell culture medium can then be harvested to obtain the produced valencene.

The valencene produced in cells expressing valencene synthase can be isolated and assessed by any method known in the art. In one example, the cell culture medium is extracted with an organic solvent to partition valencene and any other terpene produced, into the organic layer. Valencene production can be assessed and/or the valencene isolated from other products using any method known in the art, such as, for example, gas chromatography. For example, the organic layer can be analyzed by gas chromatography using cedrene and hexadecane as internal standards.

### E. ASSESSMENT OF TERPENES

The quantity and/or purity of terpenes, such as valencene and/or nootkatone produced or obtained by any of the methods described herein or known in the art, can be determined by any known standard chromatographic technique useful for separating and analyzing organic compounds. For example, terpene production can be assayed by any known chromatographic technique useful for the detection and quantification of hydrocarbons, such as terpenes, including, but not limited to, gas chromatography (GC), mass spectrometry (MS), gas chromatography using a flame ionization detector (GC-FID), capillary GC-MS, high performance liquid chromatography (HPLC) and column chromatography. Typically, these techniques are carried out in the presence of known internal standards, for example, cedrene or hexadecane, which are used to quantify the amount of the terpene produced. For example, terpenes, including sesquiterpenes, can be identified by comparison of retention times and mass spectra to those of authentic standards in gas chromatography with mass spectrometry detection. Typical standards include, but are not limited to, cedrene and hexadecane. In other examples, quantification can be achieved by gas chromatography with flame ionization detection based upon calibration curves with known amounts of authentic standards and normalization to the peak area of an internal standard.

As a product of the oxidation there can be a mixture of terpene products, such as nootkatone and nootkatol, in which the relative proportions of the two compounds can vary depending on the reaction conditions. The components can be separated from the reaction product using techniques described above. Generally, the purity of nootkatone for use in the compositions herein is at least 65%, 70%, 75%, 80%, 85%, 86%, 87%, 88%, 89%, 90%, 91%, 92%, 93%, 94%, 95%, 96%, 97%, 98%, 99% or higher. Typically, the purity of nootkatone is at least 90%.

### F. COMPOSITIONS

Compositions containing nootkatone or a derivative or analog of nootkatone are provided that can be used for controlling insects and pests, and they are active against normally sensitive and resistant species, and against all or individual developmental stages. The compositions can be used to repel, expel, incapacitate, deter, eliminate, alleviate, reduce the number of, eradicate or kill an insect or pest.

Effectiveness of any insect repellent can depend upon the surface concentration of the active ingredient on the insect/pest surface or locus to which it is applied. Many compounds known in the art to exhibit insect repellency do so only in relatively concentrated form, such as up to 25% (e.g., see U.S. Pat. No. 4,416,881, which discloses the use of repellent concentrations of 6.25-25%).

The nootkatone or derivative or analog thereof can be incorporated in effective amounts into a composition suitable for application to an animal, such as to human skin, or inanimate surface, such as a bed sheet. Suitable compositions can include a carrier, and the compositions can be formulated to provide a final product that is in the form of a lotion, gel or cream, an aerosol or spray, or can be included on a silicaceous clay, talc or granule.

The amount of nootkatone or an analog or derivative thereof that is to be included in the composition can vary, but it has been determined that compositions that contain at 0.1% to 10%, or greater than 10% nootkatone or an analog or derivative thereof are effective in controlling, including repelling and/or killing, insects or pests, such as for up to several, including four days. The amount of nootkatone or a derivative or analog thereof can be increased in order to provide faster or longer control, such as knockdown, repelling or killing, of an insect or pest. For example, compositions containing greater than 0.5%, or 1%, or 2%, or 5%, or 7.5%, or 10%, or 15%, or 20%, or 25% nootkatone and/or a derivative or analog thereof can be effective in repelling and/or killing bedbugs, fleas and lice. Higher concentrations of nootkatone or an analog or derivative thereof, such as at least at or about 15% nootkatone or an analog or derivative thereof also can be included in order to prolong the residual action of the composition or increase the effect thereof.

The compositions provided herein include at least nootkatone or an analog or derivative thereof and a carrier. The nootkatone or analog or derivative thereof can be present in an amount of from at least at or about 0.1% to at or about 10%, or greater than 10%, or greater that 15%, or greater than 20%, or greater than 25% by weight of the composition. Generally, the composition contains from about or 0.1% to about or 15% nootkatone or an analog or derivative thereof. In some compositions, the composition contains greater than 10%, or greater that 15%, or greater than 20%, or greater than 25% nootkatone or an analog or derivative thereof. In some applications, the composition contains at least 1% nootkatone or an analog or derivative thereof. In some applications, the composition contains greater than 10% nootkatone or an analog or derivative thereof. In some applications, the composition contains greater than 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, 10%, 10.05%, 10.1%, 10.15%, 10.2%, 10.25%, 10.3%, 10.35%, 10.4%, 10.45%, 10.5%, 10.55%, 10.6%, 10.65%, 10.7%, 10.75%, 10.8%, 10.85%, 10.9%, 10.95%, 11%, 11.05%, 11.1%, 11.15%, 11.2%, 11.25%, 11.3%, 11.35%, 11.4%, 11.45%, 11.5%, 11.55%, 11.6%, 11.65%, 11.7%, 11.75%, 11.8%, 11.85%, 11.9%, 11.95%, 12%, 12.05%, 12.1%, 12.15%, 12.2%, 12.25%, 12.3%, 12.35%, 12.4%, 12.45%, 12.5%, 12.55%, 12.6%, 12.65%, 12.7%, 12.75%, 12.8%, 12.85%, 12.9%, 12.95%, 13%, 13.05%, 13.1%, 13.15%, 13.2%, 13.25%, 13.3%, 13.35%, 13.4%, 13.45%, 13.5%, 13.55%, 13.6%, 13.65%, 13.7%, 13.75%, 13.8%, 13.85%, 13.9%, 13.95%, 14%, 14.05%, 14.1%, 14.15%, 14.2%, 14.25%, 14.3%, 14.35%, 14.4%, 14.45%, 14.5%, 14.55%, 14.6%, 14.65%, 14.7%, 14.75%, 14.8%, 14.85%, 14.9%, 14.95%, 15%, 15.05%, 15.1%, 15.15%, 15.2%, 15.25%, 15.3%, 15.35%, 15.4%, 15.45%, 15.5%, 15.55%, 15.6%, 15.65%, 15.7%, 15.75%, 15.8%, 15.85%, 15.9%, 15.95%, 16%, 16.05%, 16.1%, 16.15%, 16.2%, 16.25%, 16.3%, 16.35%, 16.4%, 16.45%, 16.5%, 16.55%, 16.6%, 16.65%, 16.7%, 16.75%, 16.8%, 16.85%, 16.9%, 16.95%, 17%, 17.05%, 17.1%, 17.15%, 17.2%, 17.25%, 17.3%, 17.35%, 17.4%, 17.45%, 17.5%, 17.55%, 17.6%, 17.65%, 17.7%, 17.75%, 17.8%, 17.85%, 17.9%, 17.95%, 18%, 18.05%, 18.1%, 18.15%, 18.2%, 18.25%, 18.3%, 18.35%, 18.4%, 18.45%, 18.5%, 18.55%, 18.6%, 18.65%, 18.7%, 18.75%, 18.8%, 18.85%, 18.9%, 18.95%, 19%, 19.05%, 19.1%, 19.15%, 19.2%, 19.25%, 19.3%, 19.35%, 19.4%, 19.45%, 19.5%, 19.55%, 19.6%, 19.65%, 19.7%, 19.75%, 19.8%, 19.85%, 19.9%, 19.95% or 20% nootkatone or an analog or derivative thereof by weight of the composition. The nootkatone or analog or of nootkatone or their combination an be present in an amount of up to 99% by weight of the composition.

Other than increasing the amount of the nootkatone in the composition, residual action of the composition also can be modulated by selection of the carrier and other components of the composition. For example, when the carrier includes or is a solvent, a solvent having a particular vapor pressure, or a combination of solvents having differing vapor pressures, can be used to modify the evaporative rate or vaporization rate of the nootkatone or analog derivative thereof from the composition. Other components also can be included in the composition in order to modulate the residual action of the nootkatone or analog or derivative thereof. For example, a surfactant with which the nootkatone can interact can be included in the composition, which can reduce the rate of evaporation and thereby increase the residual action of the composition. Polymers and thickeners and other viscosity modulating agents also can be included in the composition to modulate the viscosity of the composition, and thereby slow the release of the nootkatone or analog or derivative thereof from the composition or otherwise prolong the time that the insect/pest is exposed to the composition or vapors from the composition. Synergists also can be included in the composition. Examples of suitable synergists for use in the compositions include bis-(2,3,3,3-tetrachloropropyl)ether, dodecyl imidazole, N-(2-ethylhexyl)bicyclo-[2,2,1]hept-5-ene-2,3-dicarboxyimide piperonyl butoxide, isobornyl thiocyanatoacetate, safroxan and sesamex.

1. Carrier

The compositions provided herein containing nootkatone or an analog or derivative thereof include a carrier. In general, any material that can be used as a carrier for a volatile essential oil is suitable as a carrier in the compositions and formulations provided herein. The carrier generally is mixed with the nootkatone or derivative or analog thereof and generally is selected to facilitate the application of the composition to a targeted locus, such as a subject or location, or to facilitate storage, transport and/or handling of the composition. The carrier can be in the form of solid and/or liquid and/or gas, such as a propellant.

For an insect repellent to be effective, the evaporation rate of the active ingredient from the host's skin or the treated surface or locus or article must be sufficiently high to provide a vapor density that has the desired effect on the target insects or pests. There is a balance that should be considered between evaporation rate and the desired duration of the insect repellent effect. If the evaporation rate is too high, the nootkatone will be depleted from the surface rapidly, causing a loss in efficacy. There are a number of factors that can modulate evaporation rate, such as the ambient temperature, the temperature of the treated surface, and the presence or absence of air movement. These factors should be taken into consideration when formulating a product, but generally are beyond the direct control of the formulator. The compositions provided herein can be formulated to have a surface evaporation rate of at least a minimum effective evaporation rate, and generally can have a minimum effective evaporation rate that lasts at least four hours. In some compositions, particularly for application to a skin surface, the compositions can provide a minimum effective evaporation rate lasts at least 0.5, 1, 2, 3, 4, 5, 6, 7, 8, 9, 10, 11, 12, 13, 14, 15, 16, 17, 18, 19, 20, 21, 22, 23 or 24 hours or more.

One skilled in the art can determine an optimal concentration of carrier to be included in the composition or formulation. The carrier can be a fluid, which can include liquids, gases or solids, such as finely divided particulates. One skilled in the art can select the appropriate form of particular dosage preparations for specific application made therefrom, such as solutions, emulsions, suspensions, powders, pastes, and granules which are thus ready for use. The carrier can be up to 90%, or up to 99%, by weight, of the composition. In some examples, the composition can include at least at or about 10% carrier. In other examples, the composition can include carrier in any range between at or about 1% to at or about 99%, by weight, of the composition. For example, the composition can include carrier in an amount of from 10% to 80%, by weight, or 20% to 70%, by weight, or 30% to 60%, by weight, or 10% to 40%, by weight, or 60% to 90%, by weight, of the composition.

a. Liquid Carriers

In some applications, the composition and formulations provided herein include a liquid carrier. Exemplary liquid carriers include alcohols, alkanes, alkenes, aqueous solutions, cycloalkanes, esters, ethers, glycols, ketones, oils, organic solvents, silicones or silicone oils, and combinations thereof. In some applications, the liquid carrier contains up to at or about 95%, by weight, of the composition. For example, the composition can contain at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94% or 95% liquid carrier, by weight, of the composition.

In some examples, the carrier can contain an alcohol. Exemplary alcohols that can be included in the compositions and formulations provided herein are C₁-C₈ alcohols, such as methanol, ethanol, propanol, isopropanol, butanol, sec-butanol and tert-butanol, C₂-C₆ polyhydric alcohols, polyvalent alcohols, such as glycerol, and mixtures thereof. In some examples, the carrier is a glyceride, that is a monoglyceride, a diglyceride, an acetylated monoglyceride, or a triglyceride or a combination thereof.

In some examples, the carrier is an alcohol that is a 1-propanol, 2-propanol, 1-butanol, 2-butanol, 2-methyl-1-propanol, 1-pentanol, 2-pentanol, 3-methyl-1-butanol, 3-methyl-2-butanol, ethylene glycol, propylene glycol, 1,4-butanediol, 1,3-butanediol, 2-methyl-1,3-propanediol, 1,4-cyclohexanedimethanol, diethylene glycol, triethylene glycol, PEG-200, PEG-300, PEG-400, PEG-600, 2-methoxyethanol, 2-ethoxyethanol, 2-propoxyethanol, 2-isopropoxyethanol, 2-butoxyethanol, 1-methoxy-2-propanol, 1-ethoxy-2-propanol, 3-methoxy-1-butanol, diethylene glycol monomethyl ether, diethylene glycol monoethyl ether, diethylene glycol mono-n-propyl ether, diethylene glycol mono-isopropyl ether, diethylene glycol monobutyl ether, triethylene glycol monomethyl ether, glycerol, 3-methoxy-1,2-propanediol, or 3-ethoxy-1,2-propanediol. In other examples, the carrier can be a borneol, citronellol, geraniol, D-limonene, dipentene or a combination thereof.

For some insects or pests, the carrier should contain little to no isopropanol. It has been determined that, for killing or repelling some insects or pests, compositions or formulations that do not contain isopropanol are advantageous. Thus, in some applications, the amount of isopropanol in the composition or formulation does not exceed 5%, or is 4% or less, or is 3% or less, or is 2% or less, or is 1% or less, and in some formulations isopropanol is not included. Is some applications, the total amount of isopropanol in the composition or formulation is 5% or less, or 1% or less.

In some examples, the carrier also can include a C₁-C₁₀ alkane or a C₁-C₁₀ alkene. In other examples, the carrier also can include a C₁-C₈ cycloalkane. In yet other examples, the carrier can contain an ester. Exemplary esters that can be included in the provided compositions or formulations include acetate, benzoate esters, butyrate, isobutyrate, caproate, isocaproate, hexanoate, heptanoate, octanoate, phenylacetate, propionate, isopropionate, valerate and isovalerate. In other examples, the carrier can contain an ether. Exemplary ethers that can be included in the composition as a carrier include, but are not limited to, diethyl ether, isopropyl ether and n-propyl ether, and combinations thereof.

In some examples, the carrier can contain a glycol. Exemplary glycols that can be included in the composition as a carrier include, but are not limited to, butylene glycol, butylene glycol monomethyl ether, butylene glycol dimethyl ether, butylene glycol monoethyl ether, butylene glycol diethylether, ethylene glycol, ethylene glycol monomethyl ether, ethylene glycol dimethyl ether, ethylene glycol monoethyl ether, ethylene glycol diethylether, diethylene glycol, propylene glycol, propylene glycol monomethyl ether, propylene glycol dimethyl ether, propylene glycol monoethyl ether, propylene glycol diethyl ether, pentylene glycol and hexylene glycol, and combinations thereof.

In some examples, the carrier also can include a ketone. Exemplary ketones that can be include in the compositions and formulations provided herein include, but are not limited to, acetone, acetophenone, benzyl methyl ketone, cyclohexanone, ethyl ketone, 3-methylacetophenone, methyl ethyl ketone, methyl propyl ketone, methyl isopropyl ketone, methyl butyl ketone, 4-phenylcyclohexanone, and combinations thereof.

The carrier also can include an oil. Exemplary oils that can be included in the compositions and formulations provided herein include, but are not limited to, short-chain fatty acid triglycerides, silicone oils, petroleum fractions or hydrocarbons such as heavy aromatic naphtha solvents, light aromatic naphtha solvents, hydro-treated light petroleum distillates, paraffinic solvents, mineral oil, alkylbenzenes, paraffinic oils, and vegetable/plant oils and their derivatives, such as almond oil, avocado oil, canola oil, cashew oil, cherry seed oil, cocoa butter, coconut oil, corn oil, cottonseed oil, flaxseed oil, grape seed oil, jojoba oil, macadamia nut oil, olive oil, palm oil, palm fruit oil, peanut oil, rapeseed oil, rice bran oil, safflower oil, sesame oil, soybean oil, sunflower oil, and walnut oil, and alkylated vegetable oils and alkyl esters of fatty acids, such as methyloleate, and combinations thereof.

In some examples, the carrier also can include a silicone or a silicone oil. Exemplary silicones or silicone oils that can be included in the compositions and formulations as a carrier include, but are not limited to, cyclical silicones, linear, branched open chained silicones volatile silicones, such as dimethicone copolyol, cyclomethicone, polydimethylsiloxane, cyclic dimethyl polysiloxane, aminosilicones, phenylsilicones, diphenyldimethicones, phenyltrimethicones, cyclopentasiloxane, a polymer of dimethyl-siloxane with polyoxyethylene and/or polyoxypropylene, dimethicone copolyol, cetyldimethicone copolyol, cetyl dimethicone, cetyl dimethiconecopolyol and dimethiconol, and non-volatile silicones, such as cyclic polydimethylsiloxanes containing an average of from about 3 to about 9 silicon atoms and linear polydimethylsiloxanes containing an average of from about 3 to about 9 silicon atoms, polydimethylsiloxane, phenylated silicones, decamethylcyclopentasiloxane, dodecamethylcyclohexasiloxane and octamethyl-cyclotetrasiloxane and hexamethyl cyclotrisiloxane, octamethyl cyclotetrasiloxane, decamethyl cyclopentasiloxane, dodecamethyl cyclohexasiloxane, tetradecamethyl cycloheptasiloxane, hexadecamethyl cyclooctasiloxane, tetramethyl cyclotetrasiloxane, pentamethyl cyclopentasiloxane, pentamethyl cyclotetrasiloxane, hexamethyl cyclotetrasiloxane, heptamethyl cyclotetrasiloxane, hexamethyl cyclopenta-siloxane, octamethyl cyclopentasiloxane, heptamethyl cyclopentasiloxane, nonamethyl cyclopentasiloxane, cyclomethicone, trisiloxane, volatile dimethicones, polyalkylsiloxanes, polydialkylsiloxanes, methyl trimethicone, cyclopolysiloxanes, and mixtures thereof.

The oil carriers can be used to slow down early evaporation of the nootkatone or derivative or analog thereof from the composition, thereby leaving more nootkatone available for later evaporation and release into the vicinity of application. Incorporation of an oil in a composition in sufficient amounts to slow the migration of the nootkatone out of the composition when applied can increase the composition's length of effectiveness by, for example 25% to 50%, over comparable non-entrapped products. When a composition includes too much oil as a carrier, however, it can be perceived as feeling greasy.

Liquid carriers also can provide water repellency and thereby extend the effectiveness of the composition on a targeted locus, such as human skin. Some liquid carriers, particular some vegetable oils, can help to prevent skin irritation, and/or soothe and condition skin. Factors to consider when selecting a carrier(s) for the compositions provided herein include commercial availability, cost, repellency, evaporation rate, odor, and stability. Some carriers can themselves have repellent properties.

b. Gas Carriers

In some applications, the carrier in the composition or formulation provided herein is or includes a gas. Generally, the gas is provided in the form of a propellant that can be a liquid when contained in a container under pressure, and converts to a gas upon removal of the pressure. For example, halogenated or unhalogenated gaseous aliphatic hydrocarbons and mixtures thereof can be liquefied at 20° C. by use of an excess pressure of at least 0.5 atmosphere. Exemplary gas carriers for use in the compositions and formulations include, but are not limited to, aerosol propellants, such as argon, butane, carbon dioxide, a chlorofluorocarbon, such as dichlorodifluoromethane or dichlorotetrafluoroethane, dimethyl ether, a hydrocarbon, a hydrofluorocarbon, such as difluoromethane, trifluoromethane, difluoroethane, trifluoroethane, tetrafluoroethane, or octafluorocyclobutane, isobutane, nitrogen, propane, or mixtures thereof, butane, dimethyl ether, a fluorocarbon, such as Freon™ gas and carbon dioxide. The amount of gas carrier included in the compositions or formulations can vary, and can be selected based on any one of several criteria, including the physical behavior of the propellant, on the type of nozzle or device selected to deploy the composition, and on the volume of the pressure vessel used. The proportion of gas carrier in the composition or formulation can vary from between at or about 10% to at or about 90%, by weight, of the composition.

c. Solid Carriers

The carrier in the compositions provided herein also can be dispersible finely divided solid carriers, such as solid carriers having a particle size of less than about 200 microns, 100 microns, or generally less than 50 microns. Examples of solid carriers include, but are not limited to, finely divided organic solid material or finely divided inorganic solid material, for example, alumina, amorphous silica, attapulgite, calcium carbonate, calcium phosphate, clay, chalk, i.e., calcium carbonate, bentonite, fumed silica, clays, diatomaceous earth, fullers earth, kaolin, magnesium carbonate, microparticulate cellulose, montmorillonite, pyrophyllite, silicic acid, sodium bicarbonate, sodium carbonate, sodium phosphate, sodium pyrophosphate, talc, vermiculite, hydrated aluminum silicate, quartz, silica (amorphous or fumed), silicates, and smectite clay.

The carriers also can be granules of naturally occurring materials or substrates such as crushed rock or stone (calcite, marble, pumice), shredded paper or paper fibers, plant materials, such as ground corn cobs, peanut shells, saw dust or synthetic materials, such as nylon fibers. Granules can be porous or nonporous or a combination thereof. The granule particles can be of any desired size, which can be determined according to the targeted end use of the product. Generally, granules have a mean diameter of less than 5000 microns, and often are in the range of between 500 microns and 2500 microns. When granules are a part of the carrier system, the compositions containing nootkatone or a derivative or analog thereof can be applied to a surface of a nonporous granule or on the surface and/or interior of a porous granule. When a solid carrier system is used, the composition or formulation can include components that can assist the composition to adhere to the solid carrier. Such components can include any viscosity modulating agent. In some applications, a film forming agent, such as gum arabic, polyvinyl acetate, propylene glycol alginate or cellulosic material, is included in the composition or formulation to adhere the composition or formulation to the particulate carrier.

2. Additional Ingredients

In some applications, the compositions and formulations provided herein include additional ingredients, such as anti-oxidants, dispersing agents, emulsifiers, viscosity modulating agents, preservatives, colorants and synergists, or combinations thereof.

a. Anti-Oxidants

An anti-oxidant can be included in the composition, e.g., to increase the length of time the deployed composition can be exposed to the environment or to decrease any negative impact oxygen or free radical thereof can have on the compositions. Exemplary anti-oxidants that can be included in the compositions provided herein include, but are not limited to, ascorbyl palmitate, butylated p-cresol, tert-butylhydroquinone, butylated hydroquinone monomethyl ether, butylhydroxyanisole, butylhydroxytoluene, propyl gallate, tocopherol, ascorbic acid, dibutyl-hydroxy toluene, dihydroquercetin, octyl gallate, dodecyl gallate, ethoxyquin, mixed tocopherols, octadecyl-3-(3,5-ditertiarybutyl-4-hydroxy-phenyl) propionate, pentaerythritol-tetrakis[3-(3,5-ditertiarybutyl-4-hydroxyphenyl)-propionate], 2,5-ditertiary-butyl hydroquinone, 4,4′-thiobis(3-methyl-6-tertiarybutyl phenol) and 2,2′-methylene-bis-(4-methyl-6-tertiarybutyl phenol), and combinations thereof. Typically, an antioxidant can be included in the provided compositions in a protective amount, typically the lowest effective amount, such as, but are not limited to, between at or about 0.001% to at or about 5%, between at or about 0.005% to at or about 2.5%, or between at or about 0.01% to at or about 1%, by weight, of the composition.

b. Emulsifiers and Dispersing Agents

In some applications, the compositions provided herein can include an emulsifier and/or dispersing agent. The emulsifier and/or dispersing agent can serve any one or more functions in the composition. For example, an emulsifier and/or dispersing agent can help to stabilize an emulsion formed between hydrophobic and hydrophilic components of the application. An emulsifier and/or dispersing agent also can serve as a wetting agent, to enable the composition to more easily coat a substrate or targeted locus. An emulsifier and/or dispersing agent also can serve as an auxiliary to form a foam. Typically, a dispersing agent or emulsifier can be included in the provided compositions and formulations in an amount between at or about 0.002% to at or about 50%, by weight, of the composition, such as, for example, between at or about 0.025% to at or about 25%, or between at or about 0.01% to at or about 15%, by weight, of the composition.

Dispersing agents that can be included in the compositions and formulations include, for example, surfactant, polyvinylpyrrolidone, polyoxyethylated castor oil, a polyoxyethylene sorbitan ester, alkylnaphthalene sulfonate, alkylbenzenesulfonate, polyoxyethylene, polycarboxylate, lignin sulfonate, sodium silicate, potassium silicate, methylcellulose, carboxymethyl cellulose, hydroxypropylcellulose, hydroxypropyl-methylcellulose, gum arabic, a polyacrylate, and an acrylic/maleic copolymers and combinations thereof.

Emulsifiers that can be included in the compositions and formulations include, for example, ionic, non-ionic, zwitterionic and/or anionic surfactants. In some examples, the emulsifier is a surfactant that is anionic, such as sodium lauryl sulfate (USP) and its derivatives, alkyl sulfonate surfactants, a linear alkylbenzene sulfonic acid, a branched alkylbenzene sulfonic acid a C₁₂ to C₁₈ alkylsulfate, C₁₂-C₁₈ alkyl alkoxy sulfate, C₁₂-C₁₈ alkyl methyl ester sulfonate, fatty soaps, alkyl sulfates, sulfated oils, ether sulfates, sulfonates, sulfosuccinates, sulfonated amides and isethionates; a zwitterionic surfactant; cationic surfactant, such as an alkylamine, an alkyl diamine, an alkyl polyamine, a mono- or di-quaternary ammonium salt, a monoalkoxylated amine, a dialkoxylated amine, a monoalkoxylated quaternary ammonium salt, a dialkoxylated quaternary ammonium salt, an etheramine, an amine oxide, an alkoxylated amine oxide and a fatty imidazoline, quaternary ammonium halides (such as cetyl pyridinium chloride); or a non-ionic surfactant, such as linear fatty alcohol ethoxylates or their polyoxyethylene condensation products (such as Spans and Tweens), alkyl arylpolyglycol ethers, polyethylene oxide esters of fatty acids, polyglycerol esters of fatty acids, polyoxethylene sorbitan monolaurate, polyoxethylene sorbitan mono- or tri-stearate, polyoxyethylene sorbitan monoleate, propylene glycol mono and diesters of fats and fatty acids, aryl sulfonates, sorbitan monostearate, poloxamer and its derivatives, medium chain triglyceride, caprylocaproyl macrogolglycerides, diethyleneglycol monoethyl ether, PEG-6 olive oil, PEG-6 peanut oil, PEG-6 hydrogenated palm kernel oil, propylene glycol dicaprylate/dicaprate, polysorbate, sorbitan esters, polyethoxylated castor oil, PEG-60 hydrogenated castor oil, PEG-40 hydrogenated castor oil, sodium lauryl glutamate, disodium cocoamphodiacetate, Polyoxyl 23 lauryl ester, an alkoxylated alcohol, a dialkoxylated alcohol, an alkoxylated dialkylphenol, an alkylpolyglycoside, an alkoxylated alkylphenol, an alkoxylated glycol, an alkoxylated mercaptan, an alkylamine salt, an alkyl quaternary amine salt, a glyceryl or polyglyceryl ester of a natural fatty acid, an alkoxylated glycol ester, an alkoxylated fatty acid, an alkoxylated alkanolamide, a polyalkoxylated silicone and an N-alkyl pyrrolidone, and combinations thereof.

c. Viscosity Modulating Agents

In some applications, the composition can include a viscosity modulating agent. Viscosity modulating agents, such as polymers and thickeners, can be included in the formulation for any number of reasons. A viscosity modulating agent can help to stabilize a composition during shipping and storage by preventing separation of the component, or be slowing or preventing coalescence of dispersed particle or oil droplets. A viscosity modulating agent can help to stabilize an emulsion or as an auxiliary to form or stabilize a foam. A viscosity modulating agent also can modify the rheology of the composition, thereby facilitating application to a targeted locus. For example, a viscosity modulating can be selected such that the composition when dispensed from a spraying device produces even or uniform droplets with little overspray. In applications where the composition is to be applied to a locus by brushing or dipping, a viscosity modulating agent can be selected so that the composition adheres to the surface of the locus and coats the surface. The viscosity modulating agent also can modifying the release profile of the composition and thereby slow the release of the nootkatone or analog or derivative thereof from the composition.

Any viscosity modulating agent known in the art can be selected, based on the properties desired in the final composition. Some polymers have an intrinsic viscosity that they impart to a formulation based on the concentration of the viscosity modulating agent included in the composition. Lower molecular weight polymers generally tend to have lower viscosity contributing ability at lower concentrations, meaning that a higher percentage of viscosity modulating agent is required to achieve a higher viscosity in the final composition. The skilled artisan can select the type of viscosity modulating agent, its molecular weight and the percentage to include in the composition based on the chemistry and rheology of the viscosity modulating agent. For example, at or about 20% or more gum arabic or similar compound can be used to provide film forming and viscosity modifying effects, while as little as 0.2% xanthan gum can provide similar viscosities and stabilizing effects.

Exemplary viscosity modulating agents that can be included in the compositions provided herein include, but are not limited to, an acrylate, an acrylate copolymer, an alginate, an arabino-galactan, a carrageenan, a cellulosic polymer, such as any one of bacterial cellulose, carboxymethyl cellulose, ethyl cellulose, ethyl-hydroxyethylcellulose, hydroxylethyl cellulose, hydroxypropyl cellulose, hydroxypropyl methylcellulose, methyl cellulose, microparticulate cellulose and sodium carboxymethyl cellulose and combinations thereof, a ceramide, chitan, dextran, diutan, fucelleran, fucoidan, a β-glucan, a gellan gum (native or low acetate), guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan (including clarified scleroglucan), starch, starch hydroxyethyl ether, starch dextrins and a xanthan gum, such as a clarified or low acetate or high pyruvate xanthan gum, and combinations thereof.

The amount of viscosity modulating agent included in the composition can be selected based on the desired viscosity of the composition. For example, a free flowing composition can require very low amounts of viscosity modulating agents, while a composition in the form of a non-flowing viscous gel can require significantly higher amounts of viscosity modulating agents. Generally, the viscosity modulating agent can be included in an amount of between at or about 0.05% and at or about 25%, or between at or about 0.1% and at or about 10%, or between at or about 0.5% and at or about 5%, by weight, of the composition.

Under some circumstances or conditions of use, it can be desirable to reduce the rate of evaporation of nootkatone or a derivative or analog thereof. Any one of a number of different strategies can be used to reduce the evaporation rate of nootkatone if so desired. As discussed above, the nootkatone composition can include a viscosity modulating agent to thicken the composition, thereby requiring the nootkatone to migrate through the composition to the surface and then through the surface before it can evaporate. The composition can be prepared in the form of an emulsion, either an oil-in-water emulsion or water-in-oil emulsion. Interactions of the nootkatone with surfactants in the emulsions or physical separation of the oil particles can result in a slower release of the nootkatone from the composition as compared to a composition in the form of a liquid. Alternatively, an adhesive agent can be used to assist in adhering the compositions containing nootkatone or a derivative or analog thereof to the surface of a solid carrier. Interactions of the nootkatone or a derivative or analog thereof with the adhesive agent can result in a slower release of the nootkatone or a derivative or analog thereof from the composition as compared to a composition without any adhesive agents.

Any adhesive agents known in the art can be used to assist in adhering the formulation containing nootkatone to the surface of a solid carrier. For example, waxes, film-former and other polymers, such as alginate, carboxymethyl cellulose, gum arabic, shellac, methyl cellulose, pectin, polyvinyl alcohol, propylene glycol, rhamsan gum, welan gum and xanthan gum.

d. Preservatives

In some applications, the compositions provided herein can include a preservative. Any preservative known in the art can be included in the compositions. Exemplary preservatives include preservatives selected from among azoles, benzisothiazolin-3-one, benzalkonium quaternary compounds, benzyl alcohol, borates, 2-bromo-2-nitro-propane-1,3-diol, butylparaben, 5-chloro-2-methyl-4-isothiazolin-3-one, chlorphenesin, chloroxylenol, diazolidinyl urea, a dimethyl-benzylalkyl-ammonium chloride, ethylparaben, formaldehyde, glutaraldehyde, halogenated salicylanilides, hexachlorophene, hexylene glycol, isobutylparaben, isothiazolin-3-one, 2-methyl-4-isothiazoline-3-one, methylchloroisothiazolinone, methylisothiazolinone, methylparaben, monochloracetamide, neomycin sulfate, o-phenylphenol and salts thereof, phenoxyethanol, propionic acid and salts thereof, propylparaben, sodium benzoate, sorbic acid and salts thereof, tebuconazole and triazoles, and combinations thereof. Some preservatives are more potent or effective than others, as is known in the art. When present, the preservative can be present in an amount of at or about 0.001% to at or about 5%, by weight, of the composition. For example, the preservative can be present in the composition in an amount of at or about 0.005% to at or about 2.5%, by weight, of the composition or in an amount of at or about 0.01% to at or about 1%, by weight, of the composition.

e. Colorant

In some applications, the compositions provided herein can include a colorant. The colorant can provide additional visual cues to the applier of the composition, such as to help the applier see where the composition is being applied to a surface or locus and thereby identifying a coverage are and/or allowing an even distribution of the composition on a surface. Any colorant known in the art can be included in the compositions provided herein. Exemplary colorants for use in the provided compositions and formulations include, but are not limited to, dyes and pigments, such as titanium oxide, titanium dioxide, zinc oxide, white lead, zinc sulfide, aluminum oxide, iron oxide, silicon oxide, zirconium oxide, an azo-type colorant, a condensate-type colorant, a phthalocyanine-type colorant, a quinacridone-type colorant, an insoluble lake pigment, organic dyes, such as alizarin dyes, azo dyes or metal phthalocyanine dyes.

When present in the compositions, the colorant can be included at any concentration necessary to impart the desired or targeted color to the composition. In general, the colorant can be present in an amount of at or about 0.0001% to at or about 1%, by weight, of the composition. In some applications, the colorant is present in an amount of at or about 0.0005% to at or about 0.5%, by weight, of the composition.

f. Synergists In some examples, the provided compositions contain synergists that act to increase or prolong the effects of the compositions without increasing the amount of active ingredient. Examples of suitable synergists for use in the compositions include, but are not limited to, bis-(2,3,3,3-tetrachloropropyl)ether, dodecyl imidazole, N-(2-ethylhexyl)bicyclo-[2,2,1]hept-5-ene-2,3-dicarboxyimide piperonyl butoxide, isobornyl thiocyanatoacetate, safroxan and sesamex.

3. Microencapsulation

A method that can be used to alter the release of the nootkatone from the composition is to incorporate nootkatone or derivative or analog thereof that has been encapsulated or microencapsulated. In some examples, compositions or formulations provided herein include a encapsulated or microencapsulated active ingredient, such as a encapsulated or microencapsulated nootkatone or derivative or analog thereof, thereby altering the release of the active ingredient from the composition. Microencapsulation is well known in the art and is used to control the release rates of many essential oils, such as in fabric softener and deodorant formulations. For example, release of the active ingredient can be modulated or controlled by, e.g., surrounding the essential oil, such as nootkatone, in a film-forming material to form micron or sub-micron capsules containing the nootkatone. After contact with a substrate, such as human skin or a targeted locus, the capsules begin to breakdown and release the encapsulated nootkatone. The process can continue as new intact microcapsules are broken to release a new amount of nootkatone, replenishing nootkatone that can have been displaced or evaporated from the vicinity of application.

Microencapsulation is well known in the art and encapsulation can be accomplished using any known method. Microcapsules can be prepared using a range of conventional methods known to those skilled in the art for making shell capsules, such as interfacial polymerization and polycondensation. See, e.g., MICROENCAPSULATION: Methods and Industrial Applications (Benita and Simon, eds., Marcel Dekker, Inc. 1996), Lee et al. (2002) J. Microencapsulation 19(5): 559-569, U.S. Pat. Nos. 3,516,941, 4,520,142, 4,528,226, 4,681,806, 4,145,184 and 7,838,037; GB Patent No. 2,073,132; and International Patent Pub. No. WO 99/17871. It is recognized, however, that many variations with regard to materials and process steps are possible. Non-limiting examples of materials suitable for making the shell of the microcapsule include urea-formaldehyde, melamine-formaldehyde, phenol-formaldehyde, alginate, gelatin, gelatin/gum arabic blend, gellan gum, polyurethane and polyamides, or combinations thereof.

The microcapsules can be of any desired size, and the dimensions of the particles can be selected depending on the final use of the composition. For example, the microcapsules can have a mean diameter in the range from about 1 micron to about 100 microns, alternatively from about 5 microns to about 80 microns, alternatively from about 10 microns to about 75 microns, and alternatively between about 15 microns to about 50 microns. The particle size distribution can be narrow, broad or multimodal. The average shell thickness can vary, and can be from at or about 0.02 microns to at or about 5 microns, alternatively from at or about 0.02 micron to at or about 1 micron.

### G. FORMULATIONS

An exemplary composition provided herein includes nootkatone, a combination of nootkatone and an analog or nootkatone, or an analog of nootkatone, and a carrier. For example, the carrier in the composition can be present in an amount at or about 0.1% to 10%, or greater than 10%, or from at or about 90% to at or about 99%, based on the weight of the composition.

For example, provided are compositions that include from at or about 0.1% to at or about 10% nootkatone, or greater than 10%, or greater than 15%, or greater than 10%, or greater than 25% nootkatone and/or a derivative or analog thereof, from at or about 1% to at or about 90% carrier. The compositions can include other ingredients, such as anti-oxidants, emulsifiers, dispersing agents, surfactants, viscosity modulating agents, preservatives and colorants, or a combination thereof. For example, a surfactant can be present in an amount of from about 0.005% up to about 50% by weight of the composition, replacing an equivalent amount of carrier. For example, formulations designed for controlling drain flies can be formulated to contain surfactants and/or viscosity modifying agents so that the composition can be dispensed into a drain pipe, clinging to the pipe walls as it flows, thereby killing any adult or immature drain flies with which it comes into contact, and for providing an extended release of the composition for residual killing and repelling effects.

The compositions can include a derivative or analog of nootkatone in addition to or in place of the nootkatone. Any analog or derivative of nootkatone known in the art or that can be prepared can be included in the compositions provided herein. Examples of derivatives or analogs of nootkatone include, but are not limited to, nootkatone-11,12-epoxide, nootkatone-1,10-epoxide, nootkatone-1,10-11,12-diepoxide, tetrahydro-nootkatone and 1,10-dihydronootkatone and combinations thereof.

1. Sprays

The compositions provided herein containing nootkatone or a derivative or analog thereof can contain a liquid carrier and be formulated for delivery using a pump spray. In some applications, the composition includes a carrier containing an alcohol, ether, ester, ketone, aldehyde, oil or water or combinations thereof to provide a solution of low viscosity that can be dispensed using a pump spray. For example, a composition containing from at or about 1% to at or about 10%, or greater than 10%, or greater than 15%, or greater than 20% nootkatone and from at or about 50% to at or about 99% carrier, such as an alcohol, ether, ester, ketone, aldehyde, oil or water or combinations thereof by weight of the composition can be used as a spray for topical application to surfaces, such as bedding, clothing and carpeting.

In some examples, other carriers can be included in the composition. For example, an exemplary composition contains from at or about 1% to at or about 10%, or greater than 10% nootkatone and/or a derivative or analog thereof, by weight, of the composition, and the balance of the composition contains a carrier selected from among water, an alcohol, an aldehyde, an alkane, an alkene, an amide, an amine, a diglyceride, an ester, an ether, a glycol ether, a fat, a fatty acid, a glycol ester, a ketone, lanolin, mineral oil, a silicone or silicone oil, paraffin oil, a monoglyceride, a polyethylene glycol, petrolatum, a propylene carbonate, tall oils, a terpene hydrocarbon, a terpene alcohol, a triglyceride, finely divided organic solid material, finely divided inorganic solid materials and mixtures thereof.

The sprays can be formulated to include viscosity modifiers so that the composition adheres to the target location or the targeted insect/pest. The composition also can include a penetration agent when the spray is intended for application directly onto the insect or pest, such as for sprays directed to controlling ants, ticks, fleas, roaches, wasps, hornets, bed bugs or motes. Any penetration agents known in the art can be included. Exemplary penetration agents include silicone dioxide, petroleum distillate, light solvent naphtha or D-limonene or combinations thereof.

2. Dusts and Granules

The compositions provided herein containing nootkatone or a derivative or analog thereof can contain a solid carrier. Compositions including a solid carrier can be used when application to large area is desired, or when application in a dry form is desired or required. Solid carrier compositions are free-flowing and can be applied by methods known in the art, including spraying and spreading. Exemplary solid carrier compositions can include nootkatone and/or a derivative analog thereof applied directly to the surface of the solid carrier, or by making a pre-blend of the active component, e.g., nootkatone, in a liquid carrier and coating the solid carrier with the active component, e.g., nootkatone, pre-blend using any method known in the art, such as blending, mixing, or using a coating apparatus (see U.S. Pat. Nos. 5,043,090 and 5,413,795). The solid carrier can be less that 50 microns and in the form of a dust or powder, or can be larger, in the form of granules. The granules can be porous or nonporous. Generally, any amount of nootkatone and/or derivative or analog thereof can be adsorbed onto and/or into the granules. For example, formulations containing greater than 10%, or greater than 15%, or greater than 20% or greater than 25% nootkatone and/or a derivative analog thereof by weight of the composition can be prepared. An exemplary composition contains 15% nootkatone and 85% preformed granular attapulgite. For example, dusts or granules can be dispensed in areas infested with an insect or pest, or in areas suspected of harboring the insect/pest. For example, the dusts can be sprinkled over ant trails or under kitchen sinks where roaches had been observed. The dusts or granules attach to the pest/insect when it comes into contact with the dust or granule. The dusts can include waxes or viscosity modifying agents in order to increase the adhesion of the particles to the pest/insect.

3. Woven or Nonwoven Substrates

The compositions provided herein also can be deployed on a solid surface other than a dust or granule. For example, the compositions can be used to coat an absorbent or non-absorbent cellulosic, woven or non-woven fabric. Exemplary of such formulations are fabric softener dryer sheets, which are well known in the art (see, e.g., U.S. Pat. Nos. 6,574,883, 6,875,732, 6,930,082 and 7,989,413). Non-woven material typically can be formed of natural fibers such as cellulosic, plant-based, polylactic acid material, or synthetic fibers such as polyester, nylon, polypropylene, polytrimethylene terephthalate and polyethylene terephthalate, or, blends of such natural and synthetic fibers. The fibers can be formed in a sheet, typically by hydro-entanglement or needle-entanglement. In some examples, the woven or nonwoven sheet is a fabric treatment sheet.

The compositions containing nootkatone and/or a derivative analog thereof when applied to an absorbent or non-absorbent cellulosic, woven or non-woven fabric can be used in the dryer to deliver the nootkatone and/or derivative or analog thereof to clothing and bedding. The treated sheets also can be used to topically apply the compositions provided herein containing nootkatone to a surface by wiping the surface with the treated sheet. For example, the surface can be skin, hair or fur of an animal (such as in a moist towelette formulation), or can be a hard surface, such as a counter, floor, baseboard or headboard. In such formulations, the composition can include surfactants. The treated sheets also can be used to directly dispense the nootkatone to a locus, by placing the sheet containing the nootkatone composition in the locus. The amount of nootkatone and/or a derivative analog thereof applied to or contained on or within the treated absorbent or non-absorbent cellulosic, woven or non-woven fabric can be between 0.1% to 10%, or greater than 10%, or greater than 15%, or greater than 20%, or greater than 25% based on the total weight of the treated fabric. The composition can be formulated for delivery of nootkatone and/or analog or derivative of nootkatone for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days.

4. Aerosols

The compositions provided herein can contain a gas carrier, such as a gas propellant, and be formulated for use in aerosol devices. A propellant can be included in the carrier when the composition is to be used in aerosol devices. Aerosol devices are known in the art (see, e.g., U.S. Pat. Nos. 3,915,343, 3,884,828, 3,970,584, 4,062,937 and 6,415,992). Most aerosol products contain the active ingredient and the propellant. Examples of suitable gas propellants include propane, n-butane, isobutane, ethylene, dimethyl ether, nitrogen, nitrous oxide, carbon dioxide and mixtures thereof. An exemplary composition contains at or about 1% to at or about 10%, or greater than 10%, or greater than 15%, or greater than 20% nootkatone and/or a derivative or analog thereof, optionally at or about 10% to at or about 20% carrier, such as an alcohol, ester, ether, aldehyde or ketone, and the balance gas propellant.

5. Personal Care and Cosmetic Formulations

The compositions provided herein also can be included in a personal care or cosmetic composition. For example, a composition containing a 25:1, 20:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8. 1:9, 1:10, 1:11, 1:12, 1:13, 1:14, 1:15, 1:20 or 1:25 mixture of nootkatone and/or a derivative or analog thereof to a carrier, such as an alcohol, aldehyde, ketone, ester, oil or ether or combinations thereof, can be prepared and added to a standard personal care or cosmetic formulation to deliver the nootkatone and/or a derivative or analog thereof. Representative formulations to which the nootkatone and/or a derivative or analog thereof can be added include insect repellents, skin care products, hair care products, and cleansing products. Exemplary skin care products include skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products and anti-wrinkle products. Exemplary hair care products include hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays. Exemplary cleansing products include soaps, foaming bath products, hand/body/facial cleansers, astringent cleansers, anti-acne products, shampoos, body shampoos, synthetic detergent bars, shower gels and shampoos. For example, a shampoo can be prepared using 80% Just the Basics Shampoo (which contains water, sodium laureth sulfate, cocamide MEA, cocamidopropyl betaine, glycerin, tocopheryl acetate, panthenol, sodium methyl cocoyl taurate, PEG-7 glyceryl cocoate, polyquaternium-10, PPG-12-buteth-16, poly-quaternium-7, citric acid, sodium chloride, methylchloroisothiazolinone, methyl-isothiazolinone, disodium EDTA, tetrasodium EDTA, and fragrance), 10% nootkatone; 5% ethyl alcohol and 5% isopropyl alcohol. A similar shampoo can be prepared using 80% Just the Basics Shampoo, 15% nootkatone and 5% acetone. A similar shampoo can be prepared using 75% Just the Basics Shampoo, 20% nootkatone and 5% acetone.

The compositions provided herein containing nootkatone or a derivative or analog thereof can be formulated as topical formulations for applying to a surface, including skin or hair, also can be formulated to contain a nootkatone and/or a derivative or analog thereof. The formulation can be provided in any form suitable for topical application, such as an emulsion, a solution or suspension. Exemplary formulation forms include aerosols, creams, emulsions, foams, gels, lotions, ointments, pastes, solutions, sprays, suspensions, or any other formulations suitable for topical administration or combinations thereof.

Formulation of personal care products for topical application is well known in the art (see U.S. Pat. No. 5,472,686; and Flick, “Cosmetic and Toiletry Formulations Volume 8 (Cosmetic & Toiletry Formulations),” Noyes Publications (2001)). The composition can be applied to a surface as a single application or can be applied to a surface two or more times in one day or over the course of several days. The composition can be formulated for delivery of nootkatone and/or analog or derivative of nootkatone for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days. An effective amount of nootkatone and/or a derivative or analog thereof to include in a topical personal care formulation for killing or repelling insects and pests can be in the range of 0.1% to 10% or greater than 10%, or greater than 15%, or greater than 20%, or greater than 25%, or greater than 50% by weight of the personal care formulation.

6. Insect and Pest Repellents

Also provided herein are insect and pest repellent formulations that contain nootkatone and/or a derivative or analog thereof in the range of 0.1% to 10%, or greater than 10%, or greater than 15%, or greater than 20%, or greater than 25%, or greater than 50% by weight of the personal care formulation. The formulation can be provided as a liquid, an aerosol, a cream, a gel, a lotion, an oil, a spray, a soap, a detergent, a particulate or a substrate, such as a saturated woven or nonwoven cloth or infused plastic, such as is often used for a pet collar. In some applications, the nootkatone and/or a derivative or analog thereof can be mixed with a carrier prior to incorporating the composition into the formulation or onto or into a woven or nonwoven substrate. The ratio of nootkatone and/or analog thereof to carrier can vary, such as 25:1, 20:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8. 1:9, 1:10, 1:11, 1:12, 1:13, 1:14, 1:15, 1:20 or 1:25. When present, a nonwoven substrate can be a flexible sheet that includes fibers, which can be adhesively or thermally bonded. The fibers can be of any material, such as cellulose, cellulose ester, cotton, hemp, jute, linen, ramie, rayon, polyamides, polyesters polyolefins, polypropylene, polyvinyl derivatives, silk, sisal and wool and combinations thereof.

In some applications, the insect repellent composition includes a viscous or solid gel that can be used for topical application (such as in the form of a stick or paste) or for release of the nootkatone and/or derivative or analog thereof into a targeted locus. The viscous fluid or gel can be made by, e.g., incorporating 0.2 to 5% of a gelling agent, such as an agar, a carbomer, carboxyvinyl polymers, dibenzylidene alditols, carboxypoly-methylene, collagen, dextrin fatty acid esters, gelatin, hydrogenated styrene/isoprene copolymers, 12-hydroxystearic acid, κ-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters or wax, such as candelilla wax, carnauba wax, ceresin wax, microcrystalline wax paraffin wax and polyethylene wax, or combinations thereof, into a solvent, such as water, an alcohol, a ketone, an ester, an ether or an oil. An exemplary composition contains 2% carrageenan, 15% nootkatone, 5% acetone, 0.5% propylparaben, 0.5% potassium chloride and 77% water.

In some applications, the formulation can be prepared so that it forms a gel in situ. For example, sodium alginate can be used as a gelling agent and concurrent with or after application of the formulation, a solution of calcium chloride can be applied to the alginate-containing formulation, which will convert the sodium alginate into calcium alginate and thereby gel the formulation. Gelling agents that can exhibit delayed gelation are known in the art, including, but not limited to, agar, an alginate, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, carrageenans, such as κ-carrageenan, gellan gums, a lower hydroxy cellulose, pectins, polyacrylic acids, styrene-ethylene/propylene block copolymers, styrene-ethylene/butylene-styrene block copolymers, sucrose fatty acid esters and a wax and combinations thereof.

The insect repellent also can be provided as a particulate or powder. In such formulations, a portion or all of the carrier is a finely divided solid, such as an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, and vermiculite, and combinations thereof. These formulations can be applied by spraying, sprinkling or dusting.

The insect repellent composition, provided herein also can include, in addition to the nootkatone and/or a derivative or analog thereof, an active ingredient that repels insects. Examples of active ingredients that repel insects are N,N-diethyl-meta-toluamide (DEET), picaridin (2-(2-hydroxyethyl)-1-piperidinecarboxylic acid 1-methylpropyl ester), citronella oil, camphor oil, cedarwood oil, coumarin, 2-hydroxy-methylcyclohexyl acetic acid lactone, beta-alanine, 2-hydroxymethyl-cyclohexylidene acetic acid lactone, 2-hydroxy-methylcyclohexyl propionic acid lactone, p-menthane-3,8-diol, and 3-[N-butyl-N-acetyl]-aminopropionic acid ethyl ester and combinations thereof. The additional active ingredient can be present in an amount of from at or about 0.1% to at or about 25% by weight of the composition. In some applications, the additional active ingredient is DEET at a concentration of from at or about 2.5% to at or about 25% by weight of the composition. Other compositions including DEET can have varying amount of DEET, such as DEET at a concentration of from at or about 2.5% to at or about 5% or from at or about 5% to at or about 15% or from at or about 10% to at or about 20% by weight of the composition.

Any of the insect repellent composition provided herein can be used to repel and/or kill an insect selected from among Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurysternus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp. and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Ornithonyssus sylviarum), poultry red mite (Dermanyssus gallinae), Trombicula spp., Leptotrombidium akamushi, Ornithodorus hermsi, Ornithodorus turicata, Ornithonyssus bacoti, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp. and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Werneckiella spp., Lepikentron spp., Trichodectes spp. and Felicola spp.

For example, the insect repellent is particularly useful to repel and/or kill ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets. The insect repellent compositions can be formulated for delivery of nootkatone or analog of nootkatone for at least 1 hour, 2 hours, 3 hours, 4 hours, 5 hours, 6, hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days, or at least 4 months, or at least 5 months or at least 6 months or at least 7 months, or at least 8 months, or at least 9 months or at least 10 months or at least 11 months or at least 1 year.

The pest repellent can be provided as a packaged product. For example, the package can include a container holding any of the compositions provided herein containing nootkatone and/or a derivative or analog thereof, or an absorbent sheet impregnated a composition provided herein. The pest repellent can be provided as an aerosol propellant pressurized spray, which contains any one of the compositions provided herein and at least at or about 5% to 90% propellant by weight of the composition. Any propellant commonly used in the art for preparation or aerosol sprays can be included in the compositions. For example, the propellant can include carbon dioxide, nitrous oxide, propane, butane or a mixture thereof.

7. Insecticides or Pesticides

The compositions provided herein containing nootkatone or a derivative or analog thereof can be formulated as an insecticide or pesticide composition for administration directly to a surface of the insect or pest. The formulations generally are formulated so that they have sufficient viscosity to adhere to the insect or pest or to include ingredients that can assist the formulation to penetrate the exoskeleton of the insect or pest. In such formulations, an amount of a composition provided herein is included to provide an amount of nootkatone and/or derivative or analog of nootkatone is of at or about 0.1% to at or about 10%, or greater than 15%, or greater than 20%, or greater than 25%, or greater than 50%, by weight of the composition. The carrier of the composition provided herein can be a liquid or a solid. For example, when a solid particulate is selected, the carrier can include an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, or vermiculite, or any combination thereof.

The insecticide formulation also can be designed to include an adhesion agent so that it forms a viscous fluid or gel when dispensed onto the insect. For example, the carrier of the composition provided herein can contain an adhesion agent that includes at or about 0.2 to at or about 5% gelling agent, such as agar, an alginate, a carbomer, carboxyvinyl polymers, dibenzylidene alditols, carboxy-polymethylene, collagen, dextrin fatty acid esters, gelatin, hydrogenated styrene/isoprene copolymers, 12-hydroxystearic acid, κ-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. The adhesion agent can include 0.2 to 20% of a viscosity modulating agent selected from among an acrylate, an acrylate copolymer, an alginate, an arabinogalactan, a carrageenan, a cellulosic polymer, a ceramide, chitan, dextran, diutan, fucelleran, fucoidan, a β-glucan, a gellan gum, guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan, starch, starch hydroxyethyl ether, starch dextrins and a xanthan gum and combinations thereof. In some applications, the compositions can include penetration agents that help to penetrate the exoskeleton of the insect or pest. Any penetration agents known in the art can be included. Exemplary of these are silicone dioxide, petroleum distillate, light solvent naphtha or D-limonene or combinations thereof. Silicone dioxide causes small abrasions on the body of any insect or pest that comes into contact with the powder, thus allowing the compositions to penetrate the exoskeleton. Petroleum distillate, light solvent naphtha and D-limonene are solvents that can help dissolve any wax or cuticle on the exoskeleton, thereby allowing better adhesion of an aqueous-based formulation as well as better penetration through the exoskeleton.

8. Household Care Formulations

The compositions provided herein also can be included in a household care composition. For example, a composition containing a mixture of a carrier and nootkatone and/or a derivative or analog thereof, at a ratio of nootkatone and/or a derivative or analog thereof to carrier of 1:1, 2:1, 1:2, 3:1, 1:3, 4:1, 1:4, 5:1, 1:5, 6:1, 1:6, 7:1, 1:7, 8:1. 1:8. 9:1. 1:9, 10:1, 1:10, 11:1, 1:11, 12:1, 1:12, 13:1, 1:13, 14:1, 1:14, 15:1, 1:15, 20:1, 1:20, 25:1 or 1:25, can be prepared and added to a household care composition. The carrier can be a liquid or a solid. For example, the carrier can be selected from among water, an alcohol, an aldehyde, an alkane, an alkene, an amide, an amine, a diglyceride, an ester, an ether, a glycol ether, a fat, a fatty acid, a glycol ester, a ketone, lanolin, mineral oil, paraffin oil, a monoglyceride, a polyethylene glycol, petrolatum, a propylene carbonate, silicone, tall oils, a terpene hydrocarbon, a terpene alcohol, a triglyceride, finely divided organic solid material, finely divided inorganic solid materials and mixtures thereof.

Exemplary household care products include air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles. The formulations can be in any form, such as an aerosol, a bar, a cream, a gel, a liquid, a lotion, a paste, a powder, a roll-on, a sheet, a spray, a stick and a tablet form.

Particular household care products into which the compositions provided herein containing nootkatone and/or a derivative or analog thereof can be incorporated include laundry products, including cleansing compositions such as laundry detergents and fabric softening compositions. Such laundry products have been known in the art for decades (see, e.g., U.S. Pat. Nos. 2,954,347; 2,954,348; 3,707,503; 3,892,680; 3,929,663; 3,936,538; 4,009,114; 4,304,680; 4,566,980; 4,581,385; 5,425,891; 7,354,892; 7,387,992; 7,648,953; 7,863,236; 7,910,534; 7,910,538; 7,928,050; 7,951,768; and 7,994,112). Many laundry detergents are non-phosphated and can contain synthetic anionic surfactants, such as lauryl benzene sulfonic acid, alpha-olefin sulfonate, sodium lauryl sulfate, sodium lauryl ethoxylated sulfate, other alkyl benzene sulfonates, alcohol ether sulfates, and alcohol ethoxylates, polyacrylate and silicates.

Fabric softener formulations usually include one or more classes of softening or conditioning agents. One class of cationic softening or conditioning agents includes the quaternary amines, or “quats” or “quaternaries” as they are referred in the art. Exemplary quaternary amines include the monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries. These materials function to condition the dried fabrics and to reduce static cling and lint adherence, as well as to improve sheen and/or hand-feel. The formulations also can include soil-release agents, such as polyacrylic polyvinyl alcohol compositions (see, e.g., U.S. Pat. No. 3,377,249). The compositions provided herein containing nootkatone and/or a derivative or analog thereof can be included in such laundry product so that the final formulation contains from at or about 1% to at or about 10%, or greater than 10%, or greater than 15%, or greater than 20%, or greater than 25%, or greater than 50% nootkatone and/or an analog or derivative thereof by weight of the composition.

For example, any softening active agent known in the art (e.g., see U.S. Pat. Nos. 6,521,589 and 6,180,594), such as triethanolamine quaternary, diethanolamine quaternary, ACCOSOFT cationic surfactants (Stepan Chemical), or ditallow dimethyl ammonium chloride, can be made in an aqueous solution, to which is added a composition containing nootkatone and/or an analog or derivative thereof and a carrier. The softening active agent can be present in an amount of from at or about 5% to at or about 40% by weight of the product, or from at or about 10% to at or about 30% or from at or about 5% to at or about 15% weight by weight of the product. These compositions can be provided as liquids, gels or on a woven or nonwoven sheet, and can be formulated for use in the washer or dryer. Such sheets can contain detergent selected from among anionic surfactants, nonionic surfactants, zwitterionic surfactants, ampholytic surfactants and cationic surfactants and mixtures thereof, alone or in combination with a softening agent, such as a “quat” that can be any one of monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries or (C₈-C₂₄) fatty acid amides or any combination thereof.

The laundry products that include a composition provided herein to impart a concentration of nootkatone and/or an analog or derivative thereof of from at or about 1% to at or about 10%, or greater than 10%, or greater than 15%, or greater than 20%, or greater than 25%, or greater than 50% by weight of the composition, also can include other ingredients, such as an anti-static agent, a brightening agent, a bodying agent, a soil-release agent, a wrinkle-release agent or a combination thereof. Examples of anti-static agents include a tertiary amine, a quaternary amine, aluminum stearate or a combination thereof. Examples of brightening agents include hydrogen peroxide, potassium permanganate, sodium peroxide, sodium perborate, disulfonated diaminostilbene optical brightener compounds and triazole optical brightener compounds. Examples of bodying agents include carboxymethyl cellulose, hydroxyethyl-cellulose, starch, polyvinyl acetate and combinations thereof. An examples of a wrinkle release agent is polyvinyl acetate.

Another exemplary formulation is a fabric refresher spray composition, which can contain a concentration of nootkatone and/or an analog or derivative thereof of from at or about 1% to at or about 10%, or greater than 10%, or greater than 15%, or greater than 20%, or greater than 25%, or greater than 50% by weight of the composition. An exemplary formulation contains 11% nootkatone, 0.1% butylated hydroxytoluene (BHT) and 88.9% ethanol. The composition can be modified by reducing the amount of carrier, such as ethanol, to accommodate the addition of other ingredients. For example, the composition can include a cyclodextrin, such as alpha, beta, and gamma cyclodextrins, particularly beta-cyclodextrin. When sprayed on a fabric, the cyclodextrins can release an entrapped nootkatone over time, thereby providing a delayed release of the nootkatone and/or a derivative or analog thereof. Surfactant also can be included in the formulation, for example, to enhance the wettability of the composition.

Another exemplary formulation is a moist towelette product that contains a woven or nonwoven flexible substrate that has been treated with a composition provided herein, such that the towelette contains nootkatone and/or a derivative or analog of nootkatone in an amount of from 0.1 to 10%, or greater than 10%, or greater than 15%, or greater than 20%, or greater than 25%, or greater than 50% by weight of the composition. If formulated as a cleansing towelette, surfactants can be included in the composition. Generally, non-irritating surfactants are used, since the solution applied on the surface using the towelette can remain in place if is not immediately washed off. Exemplary non-irritating surfactants include cocamidopropyl betaine, coco-glucoside and decyl glucoside or combinations thereof.

In any of the household care products to which a composition containing nootkatone and/or derivative or analog thereof provided herein is incorporated, the final amount of nootkatone and/or derivative or analog thereof in the product is at or about at least 0.1%, 0.2%, 0.3%, 0.4%, 0.5%, 0.6%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, 10%, 10.05%, 10.1%, 10.15%, 10.2%, 10.25%, 10.3%, 10.35%, 10.4%, 10.45%, 10.5%, 10.55%, 10.6%, 10.65%, 10.7%, 10.75%, 10.8%, 10.85%, 10.9%, 10.95%, 11%, 11.05%, 11.1%, 11.15%, 11.2%, 11.25%, 11.3%, 11.35%, 11.4%, 11.45%, 11.5%, 11.55%, 11.6%, 11.65%, 11.7%, 11.75%, 11.8%, 11.85%, 11.9%, 11.95%, 12%, 12.05%, 12.1%, 12.15%, 12.2%, 12.25%, 12.3%, 12.35%, 12.4%, 12.45%, 12.5%, 12.55%, 12.6%, 12.65%, 12.7%, 12.75%, 12.8%, 12.85%, 12.9%, 12.95%, 13%, 13.05%, 13.1%, 13.15%, 13.2%, 13.25%, 13.3%, 13.35%, 13.4%, 13.45%, 13.5%, 13.55%, 13.6%, 13.65%, 13.7%, 13.75%, 13.8%, 13.85%, 13.9%, 13.95%, 14%, 14.05%, 14.1%, 14.15%, 14.2%, 14.25%, 14.3%, 14.35%, 14.4%, 14.45%, 14.5%, 14.55%, 14.6%, 14.65%, 14.7%, 14.75%, 14.8%, 14.85%, 14.9%, 14.95%, 15%, 15.05%, 15.1%, 15.15%, 15.2%, 15.25%, 15.3%, 15.35%, 15.4%, 15.45%, 15.5%, 15.55%, 15.6%, 15.65%, 15.7%, 15.75%, 15.8%, 15.85%, 15.9%, 15.95%, 16%, 16.05%, 16.1%, 16.15%, 16.2%, 16.25%, 16.3%, 16.35%, 16.4%, 16.45%, 16.5%, 16.55%, 16.6%, 16.65%, 16.7%, 16.75%, 16.8%, 16.85%, 16.9%, 16.95%, 17%, 17.05%, 17.1%, 17.15%, 17.2%, 17.25%, 17.3%, 17.35%, 17.4%, 17.45%, 17.5%, 17.55%, 17.6%, 17.65%, 17.7%, 17.75%, 17.8%, 17.85%, 17.9%, 17.95%, 18%, 18.05%, 18.1%, 18.15%, 18.2%, 18.25%, 18.3%, 18.35%, 18.4%, 18.45%, 18.5%, 18.55%, 18.6%, 18.65%, 18.7%, 18.75%, 18.8%, 18.85%, 18.9%, 18.95%, 19%, 19.05%, 19.1%, 19.15%, 19.2%, 19.25%, 19.3%, 19.35%, 19.4%, 19.45%, 19.5%, 19.55%, 19.6%, 19.65%, 19.7%, 19.75%, 19.8%, 19.85%, 19.9%, 19.95%, 20%, 21.05%, 21.1%, 21.15%, 21.2%, 21.25%, 21.3%, 21.35%, 21.4%, 21.45%, 21.5%, 21.55%, 21.6%, 21.65%, 21.7%, 21.75%, 21.8%, 21.85%, 21.9%, 21.95%, 22%, 22.05%, 22.1%, 22.15%, 22.2%, 22.25%, 22.3%, 22.35%, 22.4%, 22.45%, 22.5%, 22.55%, 22.6%, 22.65%, 22.7%, 22.75%, 22.8%, 22.85%, 22.9%, 22.95%, 23%, 23.05%, 23.1%, 23.15%, 23.2%, 23.25%, 23.3%, 23.35%, 23.4%, 23.45%, 23.5%, 23.55%, 23.6%, 23.65%, 23.7%, 23.75%, 23.8%, 23.85%, 23.9%, 23.95%, 24%, 24.05%, 24.1%, 24.15%, 24.2%, 24.25%, 24.3%, 24.35%, 24.4%, 24.45%, 24.5%, 24.55%, 24.6%, 24.65%, 24.7%, 24.75%, 24.8%, 24.85%, 24.9%, 24.95% or 25% nootkatone and/or analog of nootkatone.

### H. Preparation of the Compositions and Formulations

The compositions provided herein can be produced using methods known to the skilled artisan. For example, in compositions in which all of the ingredient are liquids and have similar polarities such that when combined they readily form a solution or dispersion, the compositions can be prepared by mixing the components together, such as using a paddle mixer or lightning mixer.

In some applications, one or more of the components of the composition can be solid at room temperature but melts at elevated temperatures to form a liquid. In cases where one of the components needs to be heated in order to incorporate them into the composition, the ingredients to be heated generally can be segregated from the nootkatone or derivative or analog thereof, which is volatile. For example, an ingredient to be heated can be mixed in a jacketed vessel while heating until liquified, and then the carrier and any optional components can be incorporated with constant mixing. The temperature of the resulting mixture then can be reduced to room temperature or slightly higher (such as 25° C.) and the nootkatone and/or derivative or analog thereof added with constant mixing until incorporated.

In some applications, the compositions are provided as a water-in-oil emulsion or an oil-in-water emulsion. Machines and apparatuses for making emulsions are known in the art. Examples of such equipment include colloid mills, sprocket dispersers and other embodiments of dynamic mixers, high-pressure homogenizers, pumps with downstream nozzles, valves, membranes or other narrow slit geometries, static mixers, in-line mixers using rotor-stator blades (Ultra-Turrax, inline dissolver), micro-mixing systems and ultrasonic emulsifiers.

The compositions provided herein containing nootkatone and/or a derivative or analog of nootkatone in an amount of from 0.1 to 10%, or greater than 10%, or greater than 15%, or greater than 20%, or greater than 25%, or greater than 50% by weight of the composition can be prepared in any known manner known in the art, for instance by blending the compositions with conventional liquid carriers and/or dispersible solid carriers. Dispersing and/or emulsifying agents, such as surface active agents, can be included to facilitate formulation, and if used, the amount of dispersing and/or emulsifying agents used is dictated by the nature of the composition and the ability of the agent to facilitate the dispersion of the components in the formulation. The compositions provided herein can be formulated for topical administration to a subject, or for administration to a surface or a locus to be treated.

### I. METHODS

The compositions provided herein can be used to repel and/or kill an insect or pest. The methods include providing a composition provided herein to a location, where the composition contains from at or about 0.1% to at or about 10%, or greater than 10%, or greater than 15%, or greater than 20%, or greater than 25%, or greater than 50% nootkatone and/or a derivative or analog of nootkatone; and deploying the composition at the location in an insect or pest repelling/killing amount, where the insect or pest is repelled when the insect or pest comes into contact with the composition or vapors from the composition. The deploying step can include atomizing, brushing on, coating, dipping, drenching, dripping, dusting, foaming, infusing, injecting into or onto, pouring, rolling on, scattering, spraying, spreading, sprinkling or wiping the composition onto at least a portion of the location. The amount of nootkatone required to repel and/or kill an insect or pest can be determined empirically and will depend on the targeted insect or pest. For some insects or pests, a composition containing from at or about 0.1% to at or about 10%, or greater than 10%, or greater than 15%, or greater than 20%, or greater than 25%, or greater than 50% nootkatone and/or analog or derivative of nootkatone is an insect or pest repelling/killing amount. Various modifications of the method can be made, such as modifying the amount of nootkatone and/or derivative or analog thereof in the composition. In some methods, the composition includes at or about or at least 0.5%, 0.75%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%, 10%, 10.05%, 10.1%, 10.15%, 10.2%, 10.25%, 10.3%, 10.35%, 10.4%, 10.45%, 10.5%, 10.55%, 10.6%, 10.65%, 10.7%, 10.75%, 10.8%, 10.85%, 10.9%, 10.95%, 11%, 11.05%, 11.1%, 11.15%, 11.2%, 11.25%, 11.3%, 11.35%, 11.4%, 11.45%, 11.5%, 11.55%, 11.6%, 11.65%, 11.7%, 11.75%, 11.8%, 11.85%, 11.9%, 11.95%, 12%, 12.05%, 12.1%, 12.15%, 12.2%, 12.25%, 12.3%, 12.35%, 12.4%, 12.45%, 12.5%, 12.55%, 12.6%, 12.65%, 12.7%, 12.75%, 12.8%, 12.85%, 12.9%, 12.95%, 13%, 13.05%, 13.1%, 13.15%, 13.2%, 13.25%, 13.3%, 13.35%, 13.4%, 13.45%, 13.5%, 13.55%, 13.6%, 13.65%, 13.7%, 13.75%, 13.8%, 13.85%, 13.9%, 13.95%, 14%, 14.05%, 14.1%, 14.15%, 14.2%, 14.25%, 14.3%, 14.35%, 14.4%, 14.45%, 14.5%, 14.55%, 14.6%, 14.65%, 14.7%, 14.75%, 14.8%, 14.85%, 14.9%, 14.95%, 15%, 15.05%, 15.1%, 15.15%, 15.2%, 15.25%, 15.3%, 15.35%, 15.4%, 15.45%, 15.5%, 15.55%, 15.6%, 15.65%, 15.7%, 15.75%, 15.8%, 15.85%, 15.9%, 15.95%, 16%, 16.05%, 16.1%, 16.15%, 16.2%, 16.25%, 16.3%, 16.35%, 16.4%, 16.45%, 16.5%, 16.55%, 16.6%, 16.65%, 16.7%, 16.75%, 16.8%, 16.85%, 16.9%, 16.95%, 17%, 17.05%, 17.1%, 17.15%, 17.2%, 17.25%, 17.3%, 17.35%, 17.4%, 17.45%, 17.5%, 17.55%, 17.6%, 17.65%, 17.7%, 17.75%, 17.8%, 17.85%, 17.9%, 17.95%, 18%, 18.05%, 18.1%, 18.15%, 18.2%, 18.25%, 18.3%, 18.35%, 18.4%, 18.45%, 18.5%, 18.55%, 18.6%, 18.65%, 18.7%, 18.75%, 18.8%, 18.85%, 18.9%, 18.95%, 19%, 19.05%, 19.1%, 19.15%, 19.2%, 19.25%, 19.3%, 19.35%, 19.4%, 19.45%, 19.5%, 19.55%, 19.6%, 19.65%, 19.7%, 19.75%, 19.8%, 19.85%, 19.9%, 19.95%, 20%, 21.05%, 21.1%, 21.15%, 21.2%, 21.25%, 21.3%, 21.35%, 21.4%, 21.45%, 21.5%, 21.55%, 21.6%, 21.65%, 21.7%, 21.75%, 21.8%, 21.85%, 21.9%, 21.95%, 22%, 22.05%, 22.1%, 22.15%, 22.2%, 22.25%, 22.3%, 22.35%, 22.4%, 22.45%, 22.5%, 22.55%, 22.6%, 22.65%, 22.7%, 22.75%, 22.8%, 22.85%, 22.9%, 22.95%, 23%, 23.05%, 23.1%, 23.15%, 23.2%, 23.25%, 23.3%, 23.35%, 23.4%, 23.45%, 23.5%, 23.55%, 23.6%, 23.65%, 23.7%, 23.75%, 23.8%, 23.85%, 23.9%, 23.95%, 24%, 24.05%, 24.1%, 24.15%, 24.2%, 24.25%, 24.3%, 24.35%, 24.4%, 24.45%, 24.5%, 24.55%, 24.6%, 24.65%, 24.7%, 24.75%, 24.8%, 24.85%, 24.9%, 24.95% or 25% nootkatone and/or an analog or derivative of nootkatone as an effective repelling/killing amount.

In the methods provided herein, the compositions provided herein containing nootkatone and/or a derivative or analog thereof can be applied to any surface or locus. In some methods, the composition is applied to a surface of the body of a subject, such as a human or animal. The animal can be a companion animal, such as a dog, cat, horse or rabbit or other animal kept by a human as a pet, or a domesticated or farm animal, such as a cow, cattle, bison, pig, horse, sheep, goat, turkey or chicken. In some methods, the compositions provided herein are applied to an article of clothing of a human, or to a bedding material, such as sheets, linens, blankets or pillows. In some methods, the composition is deployed by laundering an article of clothing of a human with a detergent or fabric softener or both that contains the composition provided herein that contains a nootkatone. In some methods, the composition is deployed by drying an article of clothing of a human with fabric softener that contains the composition provided herein that contains a nootkatone. The fabric softener used in the methods can be provided in the form of a liquid, a gel or a flexible woven or nonwoven sheet.

In some methods, the surface to which the composition is deployed is skin, hair or fur or an animal. The composition can deployed by applying topically to the skin, hair or fur, and the composition can be provided as an aerosol, a solution, an emulsion, an oil, a lotion, a soap, a spray, or a gel. In some methods, the composition is provided in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos.

A particular method provided herein targets insects or pests that have invaded a bedding location. In such methods, one or more of the compositions provided herein containing nootkatone and/or a derivative or analog thereof is deployed onto bedding (sheets, blankets, linen, pillows), bed boards, bed slats, a mattress, box springs, furniture, carpeting, baseboards or flooring or a combination thereof. The compositions can be deployed using any appropriate method, such as by atomizing, coating, dipping, drenching, dripping, dusting, foaming, infusing, injecting into or onto, pouring, rolling on, scattering, spraying, spreading, sprinkling or wiping. For example, in some methods deploying the composition includes spraying the composition on to the surface of bedding (sheet, linen, blanket, pillow), bed boards, bed slats, a mattress, box springs, furniture, carpeting, baseboards or flooring or a combination thereof. In some methods, the composition is injected into the interior of the targeted locus, such as the interior of a pillow, mattress, box springs, furniture, carpeting, baseboards or flooring or a combination thereof.

The locus selected for deploying the compositions provided herein can be determined based on the insect or pest targeted to be repelled or killed. For example, when the pest is a termite, a location for deploying a composition provided herein can include any wood structure, wooden object or wall space. For most insects or pest, treatment of any one or more of the following locations can be effective to repel or kill the insect or pest: an air supply duct, an attic, an awning, a basement, a cellar, a crawlspace, a deck, a dock, a garage, a hamper, a heating vent, a home foundation, a linen storage closet, a pool deck, roof tiles, a shipping container, a storage unit, a suitcase, a walkway and a wall space. In the methods provided, the compositions provided herein can be deployed by any technique known in the art, such as by atomizing, coating, dipping, drenching, dripping, dusting, foaming, infusing, injecting into or onto, pouring, rolling on, scattering, spraying, spreading, sprinkling or wiping the composition onto or into at least a portion of the location.

When the locus to be treated is a surface, the compositions provided herein can be applied by spraying, wiping or dusting the surface. In some methods, the composition is deployed by providing it in a form of an absorbent substrate (such as a woven or nonwoven fabric or cellulosic material) or gel and positioning it in the location. The compositions for use in the methods herein can be formulated to release nootkatone over a given length of time, depending on the application area. For example, in some applications, the compositions provided herein used in the methods to repel and/or kill insects or pests can be formulated for delivery of nootkatone and/or a derivative or analog of nootkatone for at least for at least 1 hour, 2 hours, 3 hours, 4 hours, 5 hours, 6, hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days, or at least 4 months, or at least 5 months or at least 6 months or at least 7 months, or at least 8 months, or at least 9 months or at least 10 months or at least 11 months or at least 1 year.

In the methods provided herein to repel/kill an insect or pest, the insect or pest can be any targeted insect or pest, such as Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurysternus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp. and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Ornithonyssus sylviarum), poultry red mite (Dermanyssus gallinae), Trombicula spp., Leptotrombidium akamushi, Ornithodorus hermsi, Ornithodorus turicata, Ornithonyssus bacoti, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp. and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Werneckiella spp., Lepikentron spp., Trichodectes spp. and Felicola spp. In particular, provided are methods to repel and/or kill an insect selected from among ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets.

Also provided are methods of repelling bedbugs, that include deploying any of the compositions provided herein that containing from at or about 0.01% to at or about 10%, or greater than 10%, or greater than 15%, or greater than 20%, or greater than 25%, or greater than 50% nootkatone and/or analog or derivative of nootkatone; and the insect or pest is repelled when the insect or pest comes into contact with the composition or vapors from the composition. In the methods provided, the composition can be deployed by applying it topically to an article of clothing of a human; or applying it topically to skin or hair of a human; or applying it topically to skin or fur of an animal. In the methods provided, the animal can be any animal, such as a bovine, canine, caprine, cervine, cricetine, feline, galline, equine, lapine, murine, musteline and ovine. The animal can be a human or a companion animal. In the methods provided, the nootkatone-containing composition provided herein can be deployed by laundering an article of clothing of a human with a detergent or fabric softener or both that contains the composition; or by drying an article of clothing of a human with a fabric softener that contains the composition. The composition can be deployed by applying to bedding (sheets, blankets, linens, pillows), bed boards, bed slats, a mattress, box springs, furniture, carpeting, baseboards or flooring or a combination thereof. The composition can be deployed by spraying the composition on to the surface of bedding, bed boards, bed slats, a mattress, box springs, furniture or carpeting; or injecting the composition into the pillow, mattress, box springs, furniture or carpeting or a combination thereof; or deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or injecting the composition into a wall space. The composition can be formulated for delivery of nootkatone and/or a derivative or analog of nootkatone for an extended period of time, such as for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days.

Also provided are methods of preventing skin injury due to biting insects or pests, where the methods include providing a composition provided herein that at or about 0.1% to at or about 10%, or greater than 10%, or greater than 15%, or greater than 20%, or greater than 25%, or greater than 50% nootkatone and/or a derivative or analog of nootkatone by weight of the composition; and applying the composition to a surface, wherein the insect or pest is repelled from the surface when it comes into contact with the composition or with vapors from the composition. The composition can be applied to the surface by atomizing, coating, dipping, drenching, dripping, dusting, foaming, infusing, injecting into or onto, pouring, rolling on, scattering, spraying, spreading, sprinkling or wiping an amount of the composition onto the surface. In some methods the surface is bedding (sheets, linens, blankets, pillows), clothing or a mattress. In some methods, the composition can be applied by washing the clothing or bedding with a composition provided herein that is provided as a detergent product or a fabric softener product or both or by drying the clothing or bedding with a composition provided herein that is provided as a fabric softener product. In some methods, the composition can be applied by atomizing, coating, dipping, drenching, dripping, dusting, foaming, infusing, injecting into or onto, pouring, rolling on, scattering, spraying, spreading, sprinkling or wiping the composition onto the surface.

The composition can be provided in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos. Particular pests that are to be repelled and/or killed include ants, bedbugs, chiggers, fleas, lice, mites, mosquitoes, roaches, scabies, and ticks.

Also provided are method for killing an insect or pest, where the methods include providing an insecticide formulation containing a composition provided herein that contains at or about 0.1% to at or about 10%, or greater than 10%, or greater than 15%, or greater than 20%, or greater than 25%, or greater than 50% nootkatone or analog of nootkatone by weight of the composition; and applying the composition to the insect or pest, whereby the insect or pest is killed. In some methods, the insecticide formulations further contains silicone dioxide, petroleum distillate, light solvent naphtha or D-limonene or combinations thereof. In the methods provided, the insecticide formulation can be formulated to form a viscous fluid or gel when dispensed and applied to the insect or pest. To accomplish this change in viscosity, any gelling or viscosity modulating agent known in the art can be included in the formulation, generally at a level of from at or about 0.2 to 5% gelling agent or at a level of about 0.2 to 20% of a viscosity modulating agent. Exemplary gelling agent include agar, an alginate, a carbomer, carboxyvinyl polymers, dibenzylidene alditols, carboxy-polymethylene, collagen, dextrin fatty acid esters, gelatin, hydrogenated styrene/isoprene copolymers, 12-hydroxy-stearic acid, κ-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. Exemplary viscosity modulating agent include an acrylate, an acrylate copolymer, an alginate, an arabinogalactan, a carrageenan, a cellulosic polymer, a ceramide, chitan, dextran, diutan, fucelleran, fucoidan, a β-glucan, a gellan gum, guar gum, gum arabic, gun ghatti, gum tragacanth, karaya gum, laminaran, locust bean gum, a methacrylate, a methyl methacrylate, modified starch, pectin, propylene glycol alginate, psyllium gum, polyvinyl pyrrolidone, rhamsan gum, scleroglucan, starch, starch hydroxyethyl ether, starch dextrins and a xanthan gum and combinations thereof.

Also provided are methods of treating a structure infested with termites, where the methods include deploying a composition provided herein that includes nootkatone and/or a derivative or analog thereof to the infested structure, where the composition kills or, repels the termites. In some methods, the concentration of the nootkatone and/or a derivative or analog thereof is between at or about 1% and at or about 10.0%, or greater than 10%, or greater than 15%, or greater than 20%, or greater than 25%, or greater than 50% by weight of the composition. Some methods include as a step removing the soil from around at least a portion of the structure to expose at least a portion of the foundation; applying to the exposed foundation any one or more of the compositions provided herein; and replacing to soil to cover the exposed foundation; wherein the composition forms a barrier to deter migration of termites into the structure.

Also provided are methods for treating a subject infested with an insect or pest, where the methods include providing any one or more of the compositions provided herein that contains at or about 0.1% to at or about 10%, or greater than 10%, or greater than 15%, or greater than 20%, or greater than 25%, or greater than 50% nootkatone and/or a derivative or analog of nootkatone by weight of the composition; and applying the composition to a surface of the subject, wherein the insect or pest is repelled from the surface or killed when it comes into contact with the composition or with vapors from the composition. The subject can be an animal, such as a human or a companion animal. The composition is applied to the skin, hair or fur to kill or repel chiggers, fleas, lice, mites, mosquitoes, roaches and scabies.

Use of pesticidal compositions provided herein containing at or about 0.1% to at or about 10%, or greater than 10%, or greater than 15%, or greater than 20%, or greater than 25%, or greater than 50% nootkatone and/or a derivative or analog of nootkatone by weight of the composition for treating a subject infested with an insect or pest can result in 100% knockdown on contact or within 5, 15, 30 or 60 minutes of contact. For some insects or pests, the pesticidal compositions provided herein can result in 90%, 95% or 100% mortality or the insect or pest. In some applications, the mortality occurs within 1 hour of application, or within 2 hours, 3 hours, 4 hours, 5 hours, 6 hours, 7 hours, 8 hours, 8 hours, 10 hours, 11 hours, 12 hours, 13 hours, 14 hours, 15 hours, 16 hours, 17 hours, 18 hours, 19 hours, 20 hours, 21 hours, 22 hours, 23 hours or 24 hours of applications.

In the methods provided herein, the composition containing at or about 0.1% to at or about 10%, or greater than 10%, or greater than 15%, or greater than 20%, or greater than 25%, or greater than 50% nootkatone and/or a derivative or analog of nootkatone by weight of the composition can be applied to the subject for a time sufficient to repel/kill the insects or pests. The amount of time to kill an insect or pest is dependent on the targeted insect or pest, but generally application to the infested area can be for 8 hours or less, such as for at or about 1, 2, 3, 4, 5, 6, 7 or 8 hours. In some methods, the composition is applied for about 30 minutes or less, such as for about 20 minutes, or 15 minutes, or 10 minutes or 5 minutes. In some methods, the composition can be applied more than once, if required. In some methods, the composition can be applied periodically as a preventative, such as every week, every two weeks, every month or every other month.

The compositions and methods provided herein are further illustrated in the following, non-limiting examples. The Examples are illustrative of various embodiments only and do not limit the claimed invention, including regarding the materials, conditions, weight ratios or process parameters recited herein.

### J. EXAMPLES

### Example 1

### 10% Nootkatone Solution for Knocking Down and Killing Bed Bugs

A direct spray formulation, containing 10% nootkatone (from Bedoukian Research, Inc., Product No. 800; Danbury, Conn.), 0.1% butylated hydroxytoluene (BHT) in isopropanol, stored at ambient temperature and humidity, was tested for efficacy for knockdown and mortality among adult bed bugs (Cimex lectularius). Adult bed bugs (ICR, Baltimore, Md.) used in this study were blood fed within 7 days of testing. The bed bugs were anesthetized with CO₂ and five groups of ten bed bugs were transferred into 9 dram vials at least 2 hours prior to testing. Just prior to treatment, the bed bugs were transferred to 16 oz. paper solo cups with cloth glued to the bottom on the inside of the cup and a screened lid. Each replicate was then sprayed manually, using a 2-3 oz. pump sprayer, with 1 gram±0.1 grams of the 10% nootkatone formulation. Each replicate was sprayed separately from a vertical distance of 6 inches above the bed bugs. The spray bottle was weighed before and after treating each replicate to confirm the weight of the test sample dispensed. All spraying took place in a vented, stainless steel Peet Grady chamber (6×6×6 ft). Sprayed bed bugs were observed for knockdown at 0.5, 1, 2, 3, 4, and 5 minutes post treatment. Knockdown was defined as the inability of a bed bug to right itself when placed on its back. After the five minute time point, the bed bugs were transferred to recovery containers that hold 16 oz. paper solo cups equipped with screened lids. A further knockdown study took place at 15 minutes post treatment. Serving as control samples, five replicates of 10 adult bed bugs were treated with isopropyl alcohol alone and five replicates of 10 adult bed bugs were treated with water, following the protocol outlined above. The averages of the number of bed bugs exhibiting knockdown following treatment with water, isopropyl alcohol, or 10% nootkatone, in 5 replicate trials, are set forth below in Table 3.

TABLE 3
10% nootkatone-induced knockdown of bed bugs

| Time | # Bed | Water | Isopropanol | 10% |
|---|---|---|---|---|
| post treatment (min) | bugs¹ | control¹ | control¹ | nootkatone¹ |
| 0.5 | 10 | 0 | 0 | 1.6 |
| 1 | 10 | 0 | 0 | 2.6 |
| 2 | 10 | 0 | 0 | 4.6 |
| 3 | 10 | 0 | 0 | 5 |
| 4 | 10 | 0 | 0 | 4.6 |
| 5 | 10 | 0 | 0 | 4.8 |
| 15 | 10 | 0.2 | 7.8 | 4.4 |

¹Each result presented is the average bed bug count of 5 trials.

10% nootkatone treatment showed some knockdown activity at early time points, but at 15 minutes, 10% nootkatone in the presence of isopropanol had no greater effect on bed bug knockdown than that observed for isopropanol alone. No mortality was observed for any of the treatment conditions within the 15 minute time period of the knockdown study.

After the knockdown counts were complete, the bed bugs were maintained in treatment chambers, in the laboratory at ambient light, temperature, and humidity conditions. Mortality counts were made at 24 and 48 hours post treatment. Dead bugs were confirmed by probing or agitating to make sure they were unable to move; bugs, appearing dead, that showed movement visible to the naked eye following perturbation were recorded as moribund; and bed bugs capable of crawling or righting themselves when placed on their backs were recorded as alive. Table 4 below provides the average mortality data for the five replicates of bed bugs treated with water, isopropanol alone, or with 10% nootkatone in isopropanol at 24 and 48 hours.

TABLE 4
10% nootkatone-induced mortality of bed
bugs at 24 and 48 hr post treatment.

| | 24 hr¹ | | | 48 hr¹ | | |
|---|---|---|---|---|---|---|
| Treatment | Alive | Moribund | Dead | Alive | Moribund | Dead |
| Water | 8.2 | 0.2 | 1.6 | 5.8 | 0 | 4.2 |
| Isopropanol | 4.2 | 1.2 | 4.6 | 3.2 | 0.8 | 6 |
| 10% nootkatone in | 6.4 | 0 | 3.6 | 5.2 | 0 | 4.8 |
| isopropanol | | | | | | |

¹Each result presented is the average bed bug count of 5 trials.

Treatment of bed bugs with 10% nootkatone in isopropanol did not kill bed bugs after 24 or 48 hours above mortality levels observed for isopropanol alone.

### Example 2

### 5% Nootkatone Solution for Knocking Down and Killing Bed Bugs

A direct spray formulation, containing 5% nootkatone (from Bedoukian Research, Inc., Product No. 800; Danbury, Conn.), 0.1% butylated hydroxytoluene (BHT) in acetone, stored at ambient temperature and humidity, was tested for efficacy for knockdown and mortality among adult bed bugs (Cimex lectularius) using the same procedure as described in Example 1 above. Five replicates of ten bed bugs were sprayed with 1.0 g±20% of a 5% nootkatone in acetone solution stabilized with 0.1% BHT. An additional five replicates were treated with 1.0 g±20% of tap water to serve as a control. The bed bugs were observed for knockdown at 0.5, 1, 2, 3, 4, 5 and 15 minutes. 5% nootkatone in acetone treatment showed some knockdown activity at early time points, but at 15 minutes, 5% nootkatone in the presence of acetone had no greater effect on bed bug knockdown than that observed for the control. No mortality was observed for any of the treatment conditions within the 15 minute time period of the knockdown study.

After the knockdown counts were complete, the bed bugs were maintained in treatment chambers, in the laboratory at ambient light, temperature, and humidity conditions. Mortality counts were made at 1, 2, 3, 4 and 5 days post treatment. Dead bugs were confirmed by probing or agitating to make sure they were unable to move; bugs, appearing dead, that showed movement visible to the naked eye following perturbation were recorded as moribund; and bed bugs capable of crawling or righting themselves when placed on their backs were recorded as alive. Table 5 below provides the mortality data for the replicates of bed bugs treated with water (control) or with 5% nootkatone in acetone.

TABLE 5
5% nootkatone-induced mortality of bed bugs

| | Alive | | Moribund | | Dead | |
|---|---|---|---|---|---|---|
| Minutes | Control | Treatment | Control | Treatment | Control | Treatment |
| 1 | 49 | 50 | 1 | 0 | 0 | 0 |
| 2 | 46 | 49 | 0 | 0 | 4 | 1 |
| 3 | 42 | 48 | 3 | 1 | 5 | 1 |
| 4 | 40 | 45 | 1 | 3 | 9 | 2 |
| 5 | 38 | 42 | 2 | 3 | 10 | 5 |

| | |
|---|---|
| T-test of number of live bedbugs in the | 0.005767 |
| control group versus the treatment group: | |

Treatment of bed bugs with 5% nootkatone in acetone did not kill bed bugs after 5 days above mortality levels observed for the control.

### Example 3

### Nootkatone as a Repellent for Bed Bugs

### A. Bed Bug Repellency of 10% Nootkatone in Isopropanol

A formulation, containing 10% nootkatone, 0.1% butylated hydroxytoluene (BHT) in isopropanol was tested for efficacy for repelling adult bed bugs (Cimex lectularius). Treatment chambers were created by cutting a circular hole in the bottom of 15-cm Petri dishes. A fine mesh nylon cloth was then glued to Petri dishes, covering the holes. The chambers were formed using the inverted Petri dishes with the screened bottoms serving as the tops and the lids forming the base of the chambers. The lids were secured with rubber bands.

Five semi-circular pieces of filter paper, with a diameter of 15 cm, were arranged so that they were evenly spaced on a sheet of aluminum foil. Equal amounts of 10% nootkatone solution, stored at ambient temperature and humidity, were applied to the filter papers until they were wet to the point of runoff. Five untreated semi-circular pieces were marked with the letter “c”, for control, on both sides in pencil or solvent resistant ink. One nootkatone-treated filter paper and one untreated filter paper were placed with the flat sides against each other, creating a full circle, in the lids of five 15-cm Petri dishes, serving as the treatment chambers described above, such that the entire surface of the lid was covered. Immediately after placing the filter paper semi-circles into the lid, 10 bed bugs, having received a blood meal no more than 7 days before, were placed onto the untreated piece of paper and covered with the ventilated base of the Petri dish. The containers of each replicate were then kept at ambient laboratory temperature and humidity for the duration of the study. The distribution of the bed bugs on the treated and untreated filter papers was recorded at 0.5, 1, 1.5, 2, and 24 hours after being placed in the containers. Observations were made in darkness with the aid of a red lens flashlight. Gloves and a dust mask were worn by the observer to reduce detection of the bed bugs and to minimize disturbance caused by the observer. Five additional treatment chambers were created as described above, except the treated filter paper was soaked with isopropyl alcohol (control treatment), instead of the 10% nootkatone solution. Bed bug distribution in the control chambers were recorded as described for the treatment condition.

The nootkatone treatment repellency was calculated from the following equation: R=[(C−T)/C]*100, where R is the % repellency, C is the fraction of bed bugs on the isopropanol-treated filter papers, and T is the fraction of bed bugs on the nootkatone-treated filter papers. Results indicating the efficacy of 10% nootkatone for repelling bed bugs is set forth in Table 6 below.

TABLE 6
10% Nootkatone and isopropyl alcohol-induced bed bug repellency

| | Control¹ | | | Treated¹ | | | |
|---|---|---|---|---|---|---|---|
| | # | Isopro- | Un- | # | | Un- | |
| Time | Bed | panol | treat- | Bed | 10% | treat- | Nootkatone |
| (hr) | bugs | control | ed | bugs | Nootkatone | ed | Repellency² |
| 0.5 | 10 | 2.8 | 7.2 | 10 | 0.4 | 9.6 | 85.7% |
| 1 | 10 | 3.0 | 7.0 | 10 | 0 | 10 | 100.0% |
| 1.5 | 10 | 3.4 | 6.6 | 10 | 0 | 10 | 100.0% |
| 2 | 10 | 2.2 | 7.8 | 10 | 0 | 10 | 100.0% |
| 24 | 10 | 3.6 | 6.4 | 10 | 0 | 10 | 100.0% |

Each result presented in this table is the average bed bug count of five trials. Nootkatone repellency was corrected to account for repellency observed for the control group (isopropanol alone) as described in the text.

The results from the experiment above indicate that nootkatone, at 10% in isopropyl alcohol, repels bed bugs to a greater extent than that observed for isopropanol alone.

### B. Bed Bug Repellency of 5% Nootkatone in Acetone

A formulation, containing 5% nootkatone, 0.1% butylated hydroxytoluene (BHT) in acetone was tested for efficacy for repelling adult bed bugs (Cimex lectularius) using the methods as described above. Results indicating the efficacy of 5% nootkatone for repelling bed bugs is set forth in Table 7 below.

TABLE 7
Bed bug repellency of 5% Nootkatone in acetone

| | Control | | | Treated | | | |
|---|---|---|---|---|---|---|---|
| | # | Acetone | Blank | # | | Un- | |
| Time | Bed | con- | con- | Bed | 10% | treat- | Nootkatone |
| (hr) | bugs | trol | trol | bugs | Nootkatone | ed | Repellency |
| 0.5 | 50 | 28 | 22 | 50 | 0 | 50 | 100.0% |
| 1 | 50 | 28 | 22 | 50 | 0 | 50 | 100.0% |
| 1.5 | 50 | 33 | 17 | 50 | 0 | 50 | 100.0% |
| 2 | 50 | 33 | 17 | 50 | 0 | 50 | 100.0% |
| 24 | 50 | 21 | 29 | 50 | 0 | 50 | 100.0% |

| | | | | | |
|---|---|---|---|---|---|
| Averages = | 28.6 | 21.4 | 0 | 50 | 100.0% |

The results from the experiment above indicate that nootkatone, at 5% in acetone, repels bed bugs to a greater extent than that observed for acetone alone.

C. Extended bed bug repellency of 1% and 0.25% nootkatone in ethanol

The duration of 1% and 0.25% nootkatone in isopropanol repellent effects was evaluated in this example. Bed bug (Cimex lectularius) treatment chambers were created as described in part A above, except using 9-cm Petri dishes and semicircles of filter paper with a diameter of 9 cm. Five replicate semicircles of filter paper were treated with 1 ml 1.0% nootkatone, 0.1% BHT in ethanol; 0.25% nootkatone, 0.1% BHT in ethanol; or ethanol alone (control), and placed side by side with untreated filter papers (marked with a “c”) in Petri dishes so that the lid surfaces were completely covered as previously described in part A. Prior to addition of the bed bugs, the filter papers were confirmed to be completely dry and free of any residual ethanol odor.

Following chamber preparation, 10 bed bugs, having received a blood meal no more than 7 days prior, were added to each chamber on the untreated filter paper as described above. Distribution of the 10 bed bugs per chamber was recorded at 0.5, 1, 1.5, 2, and 24 hours following treatment (Day 1). At the end of 24 hours, the old bed bugs were removed from the Petri dish and 10 new bed bugs were added. The distribution of the new set of bed bugs was recorded at 0.5, 1, 1.5, 2, and 24 hours after their introduction to the chamber (Day 2). The procedure was repeated every day for 7 days for samples treated with 1% nootkatone and 4 days for samples treated with 0.25% nootkatone. All observations were made in darkness with the aid of a red lens flashlight. Gloves and a dust mask were worn by investigators, to minimize bed bug disturbance by the observer. Percent repellency over time, was calculated and corrected using the equation: R=[(C-T)/C]*100, where R is the % repellency, C is the fraction of bed bugs on the ethanol control-treated filter papers, and T is the fraction of bed bugs on the nootkatone-treated filter papers at each time point. Corrected repellency measuring at least 75% was considered to be effective repellency.

The time courses of bed bug repellency in response to 1% and 0.25% nootkatone in ethanol treatments are set forth in Tables 8 and 9 below, respectively.

TABLE 8
1% nootkatone and ethanol induced bed bug repellency.

| | Control¹ | | | Treated¹ | | | Corrected |
|---|---|---|---|---|---|---|---|
| | # | | Un- | # | | Un- | 1% |
| | Bed | Ethanol | treat- | Bed | 1% | treat- | nootkatone |
| Day | bugs | control | ed | bugs | Nootkatone | ed | repellency² |
| 1 | 10 | 3.52 | 6.48 | 10 | 0.24 | 9.76 | 93% |
| 2 | 10 | 5.44 | 4.56 | 10 | 1.84 | 8.16 | 66% |
| 3 | 10 | 4.64 | 5.36 | 10 | 0.88 | 9.12 | 81% |
| 4 | 10 | 3.56 | 6.44 | 10 | 0.56 | 9.44 | 84% |
| 5 | 10 | 4.96 | 5.04 | 10 | 1.52 | 8.48 | 68% |
| 6 | 10 | 2.84 | 7.16 | 10 | 2.24 | 7.76 | 20% |
| 7 | 10 | 5.04 | 4.96 | 10 | 1.84 | 8.16 | 63% |

Each result presented in this table is the average bed bug count of five trials. Nootkatone repellency was corrected to account for repellency observed for the control group (isopropanol alone) as described in the text.

TABLE 9
0.25% nootkatone and ethanol induced bed bug repellency.

| | Control | | | Treated | | | Corrected |
|---|---|---|---|---|---|---|---|
| | # | | Un- | # | | Un- | 0.25% |
| | Bed | Ethanol | treat- | Bed | 0.25% | treat- | nootkatone |
| Day | bugs | control | ed | bugs | Nootkatone | ed | repellency |
| 1 | 10 | 3.52 | 6.48 | 10 | 2.68 | 7.32 | 19% |
| 2 | 10 | 5.44 | 4.56 | 10 | 4.2 | 5.8 | 23% |
| 3 | 10 | 4.64 | 5.36 | 10 | 2.32 | 7.68 | 49% |
| 4 | 10 | 3.56 | 6.44 | 10 | 4 | 6 | −14% |

Each result presented in this table is the average bed bug count of five trials. Nootkatone repellency was corrected to account for repellency observed for the control group (isopropanol alone) as described in the text.

The distribution of bed bugs in the above experiments demonstrated that 0.25% nootkatone in ethanol was not sufficient to repel bed bugs. Treatment of the filter paper with 1% nootkatone in ethanol, however, did repel bed bugs for a duration of about 4 days following treatment.

### D. Extended Bed Bug Repellency of 0.5%, 0.1 and 0.01% Nootkatone in Acetone

The duration of 0.5%, 0.1% and 0.01% nootkatone in isopropanol repellent effects was evaluated using the methods as described above.

Distribution of the 10 bed bugs per chamber was recorded at 0.5, 1, 1.5, 2, and 24 hours following treatment (Day 1). At the end of 24 hours, the old bed bugs were removed from the Petri dish and 10 new bed bugs were added. The distribution of the new set of bed bugs was recorded at 0.5, 1, 1.5, 2, and 24 hours after their introduction to the chamber (Day 2). The results are shown in Tables 10 through 16.

TABLE 10
Nootkatone at 0.50% and acetone stabilized
with 0.1% BHT, bedbug repellency (Day 1)

| | Controls | | | Treatment | | | | |
|---|---|---|---|---|---|---|---|---|
| | # | | | # | | | Repellency (%) | |
| Time | Bed | | | Bed | Nootkatone | Untreated | Control | Treatment |
| hour) | bugs | Acetone | blank | bugs | treatment | control | Group | Group |
| 0.5 | 50 | 28 | 22 | 50 | 0 | 50 | 44.0% | 100.0% |
| 1.0 | 50 | 28 | 22 | 50 | 0 | 50 | 44.0% | 100.0% |
| 1.5 | 50 | 33 | 17 | 50 | 0 | 50 | 34.0% | 100.0% |
| 2.0 | 50 | 33 | 17 | 50 | 0 | 50 | 34.0% | 100.0% |
| 24.0 | 50 | 21 | 29 | 50 | 0 | 50 | 58.0% | 100.0% |
| | Average = | 28.6 | 21.4 | | 0.0 | 50.0 | 42.8% | 100.0% |

TABLE 11
Nootkatone at 0.50% and acetone stabilized
with 0.1% BHT, bedbug repellency (Day 2)

| | Control | | | Treatment | | | | |
|---|---|---|---|---|---|---|---|---|
| | # | | | # | | | Repellency (%) | |
| Time | Bed | | | Bed | Nootkatone | Untreated | Control | Treatment |
| hour) | bugs | Acetone | blank | bugs | treatment | control | Group | Group |
| 0.5 | 50 | 20 | 30 | 50 | 7 | 43 | 60.0% | 86.0% |
| 1.0 | 50 | 23 | 27 | 50 | 6 | 44 | 54.0% | 88.0% |
| 1.5 | 50 | 30 | 20 | 50 | 3 | 47 | 40.0% | 94.0% |
| 2.0 | 50 | 29 | 21 | 50 | 1 | 49 | 42.0% | 98.0% |
| 24.0 | 50 | 22 | 28 | 50 | 4 | 46 | 56.0% | 92.0% |
| | Average = | 24.8 | 25.2 | | 4.2 | 45.8 | 50.4% | 91.6% |

TABLE 12
Nootkatone at 0.10% and acetone stabilized
with 0.1% BHT, bedbug repellency (Day 1)

| | Control | | | Treatment | | | | |
|---|---|---|---|---|---|---|---|---|
| | # | | | # | | | Repellency (%) | |
| Time | Bed | | | Bed | Nootkatone | Untreated | Control | Treatment |
| hour) | bugs | Acetone | blank | bugs | treatment | control | Group | Group |
| 0.5 | 50 | 28 | 22 | 50 | 3 | 47 | 44.0% | 94.0% |
| 1.0 | 50 | 28 | 22 | 50 | 4 | 46 | 44.0% | 92.0% |
| 1.5 | 50 | 33 | 17 | 50 | 5 | 45 | 34.0% | 90.0% |
| 2.0 | 50 | 33 | 17 | 50 | 3 | 47 | 34.0% | 94.0% |
| 24.0 | 50 | 21 | 29 | 50 | 1 | 49 | 58.0% | 98.0% |
| | Average = | 28.6 | 21.4 | | 3.2 | 46.8 | 42.8% | 93.6% |

TABLE 13
Nootkatone at 0.10% and acetone stabilized
with 0.1% BHT, bedbug repellency (Day 2)

| | Control | | | Treatment | | | | |
|---|---|---|---|---|---|---|---|---|
| | # | | | # | | | Repellency (%) | |
| Time | Bed | Acetone | blank | Bed | Nootkatone | Untreated | Control | Treatment |
| hour) | bugs | Control | control | bugs | treatment | control | Group | Group |
| 0.5 | 50 | 20 | 30 | 50 | 14 | 36 | 60.0% | 72.0% |
| 1.0 | 50 | 23 | 27 | 50 | 15 | 35 | 54.0% | 70.0% |
| 1.5 | 50 | 30 | 20 | 50 | 13 | 37 | 40.0% | 74.0% |
| 2.0 | 50 | 29 | 21 | 50 | 15 | 35 | 42.0% | 70.0% |
| 24.0 | 50 | 22 | 28 | 50 | 9 | 41 | 56.0% | 82.0% |
| | Average = | 24.8 | 25.2 | | 13.2 | 36.8 | 50.4% | 73.6% |

TABLE 14
Nootkatone at 0.01% and acetone stabilized
with 0.1% BHT, bedbug repellency (Day 1)

| | Control | | | Treatment | | | | |
|---|---|---|---|---|---|---|---|---|
| | # | | | # | | | Repellency (%) | |
| Time | Bed | Acetone | blank | Bed | Nootkatone | Untreated | Control | Treatment |
| hour) | bugs | Control | control | bugs | treatment | control | Group | Group |
| 0.5 | 50 | 28 | 22 | 50 | 16 | 34 | 44.0% | 68.0% |
| 1.0 | 50 | 28 | 22 | 50 | 17 | 33 | 44.0% | 66.0% |
| 1.5 | 50 | 33 | 17 | 50 | 17 | 33 | 34.0% | 66.0% |
| 2.0 | 50 | 33 | 17 | 50 | 14 | 36 | 34.0% | 72.0% |
| 24.0 | 50 | 21 | 29 | 50 | 21 | 29 | 58.0% | 58.0% |
| | Average = | 28.6 | 21.4 | | 17.0 | 33.0 | 42.8% | 66.0% |

TABLE 15
Nootkatone at 0.01% and acetone stabilized with 0.1% BHT, bedbug repellency (Day 2)

| | Control (note 1) | | | Treatment (note 1) | | | | |
|---|---|---|---|---|---|---|---|---|
| | # | | | # | | | Repellency (%) | |
| Time | Bed | Acetone | blank | Bed | Nootkatone | Untreated | Control | Treatment |
| (hour) | bugs | Control | control | bugs | treatment | control | Group | Group |
| 0.5 | 50 | 20 | 30 | 50 | 9 | 41 | 60.0% | 82.0% |
| 1.0 | 50 | 23 | 27 | 50 | 14 | 36 | 54.0% | 72.0% |
| 1.5 | 50 | 30 | 20 | 50 | 19 | 31 | 40.0% | 62.0% |
| 2.0 | 50 | 29 | 21 | 50 | 18 | 32 | 42.0% | 64.0% |
| 24.0 | 50 | 22 | 28 | 50 | 19 | 31 | 56.0% | 62.0% |
| | Average = | 24.8 | 25.2 | | 15.8 | 34.2 | 50.4% | 68.4% |

TABLE 16
Summary of Bedbug Repellency of Nootkatone at 0.5%,
0.1% and 0.01% and acetone stabilized with 0.1% BHT

| | | | Nootkatone | | Nootkatone | | Nootkatone | |
|---|---|---|---|---|---|---|---|---|
| | Acetone Control | | (0.50% in acetone) | | (0.10% in acetone) | | (0.010% in acetone) | |
| Day | Repellency | T-test | Repellency | T-test | Repellency | T-test | Repellency | T-test |
| 1 | 42.8% | 0.1778 | 100.0% | 0.0002 | 93.6% | 0.0001 | 66.0% | 0.0230 |
| 2 | 50.4% | 0.9246 | 91.6% | 0.0022 | 73.6% | 0.0045 | 68.0% | 0.0044 |

From the results, it can be concluded that 0.5% nootkatone in acetone is a good bedbug repellent for two days, that 0.1% nootkatone in acetone is a good bedbug repellent for one day and a moderate (statistically significant) repellent at day two, and that 0.01% nootkatone in acetone demonstrates moderate bedbug repellency for day 1 and day 2 that is statistically significant versus the control.

### Example 4

### 10% Nootkatone Solution for Knocking Down and Killing Cat Fleas

A direct spray formulation, containing 10% nootkatone, 0.1% butylated hydroxytoluene (BHT) in isopropanol, stored at ambient temperature and humidity, was tested for efficacy for knockdown and mortality among adult cat fleas (Ctenocephalides felis). Five replicates of 10 adult cat fleas (El Labs Inc (Soquel, Calif.)) were emptied from 9 dram vials into 5 gallon HDPE Payliner® pail liners lined with paper towels. Each replicate was sprayed manually with 1 gram±0.1 grams of the 10% nootkatone formulation from the rim of the pail liner. The spray bottle was weighed before and after treating each replicate to confirm the weight of the test sample dispensed. Five control replicates of 10 adult cat fleas were treated with isopropyl alcohol, following the same protocol. The study provided a single treatment of 10% nootkatone formulation or isopropyl alcohol. All spraying took place in a vented, stainless steel Peet Grady chamber (6×6×6 ft).

The average weight of isopropanol and 10% nootkatone formulation applied to the five replicates was 1.0 g and 1.07 g, respectively. Control and 10% nootkatone-treated fleas were observed for knockdown at 0.5, 1, 2, 3, 4, 5, and 15 minutes. Knockdown was defined as the failure of the cat flea to jump even following exhalation of a technician into the pail liner. Results demonstrating the efficacy of knocking down cat fleas with 10% nootkatone are set forth in Table 17 below.

TABLE 17
10% nootkatone-induced knockdown of cat fleas

| | | Isopropanol | 10% |
|---|---|---|---|
| Time (min) | # Bed Bugs¹ | control¹ | Nootkatone¹ |
| 0.5 | 10 | 0 | 0.2 |
| 1 | 10 | 0 | 0.2 |
| 2 | 10 | 0 | 0.4 |
| 3 | 10 | 0 | 0 |
| 4 | 10 | 0 | 0.2 |
| 5 | 10 | 0 | 0.4 |
| 15 | 10 | 0 | 0 |

¹Each result presented is the average cat flea count of 5 trials.

Following the knockdown studies, the payliners containing cat fleas were kept at ambient temperature and humidity in the laboratory. Mortality of cat fleas following isopropanol (control) and 10% nootkatone treatments was then recorded 24 hours after solution administration. Dead fleas were confirmed by probing the fleas or exhaling into the payliner to ensure lack of movement. Any movement visible to the naked eye, that did not include jumping, was recorded as moribund. Cat fleas that were able to jump were recorded as being alive. The number of moribund and dead fleas per replicate were added together for a total mortality count and divided by the number of fleas tested to derive percent mortality. The results are set forth in Table 18 below.

TABLE 18
10% nootkatone-induced mortality of cat fleas at 24 hours

| Treatment | Alive¹ | Moribund¹ | Dead¹ | Mortality |
|---|---|---|---|---|
| Isopropanol | 9.8 | 0 | 0.2 | 2% |
| 10% nootkatone in | 0 | 0.4 | 9.6 | 100% |
| isopropanol | | | | |

¹Each result presented is the average cat flea count of 5 trials.

In summary, nootkatone treatment was not effective in knocking down cat fleas, because 10% nootkatone knocked down no more than 4% of the fleas within the first 5 minutes, and no fleas were knocked down at 15 minutes. 10% nootkatone treatment, however, resulted in 100% mortality by 24 hours.

### Example 5

### 10% Nootkatone as a Repellent for Cat Fleas

A formulation, containing 10% nootkatone, 0.1% butylated hydroxytoluene (BHT) in isopropanol was tested for efficacy for repelling adult cat fleas (Ctenocephalides felis). Equal amounts of 10% nootkatone solution, stored at ambient temperature and humidity, were applied to five semi-circular pieces of paper towels, arranged so that they were evenly spaced on a sheet of aluminum foil, until sufficiently wet to the touch. Five untreated semi-circular pieces were marked with the letter “c”, for control, on both sides in pencil or solvent resistant ink. Each of the semi-circular pieces of paper towel had the diameter of a 5-gallon payliner. Immediately after spraying the paper towels with the nootkatone solution, the treated towels were placed in 5 5-gallon payliners. The untreated semi-circular paper towels were also placed in the payliners, with the flat sides of the semi-circle side-by-side with those of the treated pieces, so that the bottoms of the payliners were completely covered with paper towel, half treated and half untreated. Five additional payliners with one semi-circular paper towel treated with isopropyl alcohol and the other untreated served as controls. A 9 dram vial, containing 10 cat fleas (El Labs Inc (Soquel, Calif.)) was emptied onto each of the untreated paper towels in the payliners (total of 10 payliners). The distribution of the cat fleas on the control vs. treated paper towels was recorded at 0.5, 1, 1.5, 2, and 24 hours. Observations were made quietly to minimize the disturbance to the fleas by the observer. The containers were kept at ambient temperature and humidity for the duration of the study. The repellency was calculated from the following equation: R=[(C−T)/C]*100, where R is the % repellency, C is the fraction of fleas on the isopropanol-treated paper towel pieces, and T is the fraction of fleas on the nootkatone-treated paper towel pieces. Results indicating the efficacy of 10% nootkatone for repelling cat fleas is set forth in Table 19 below.

TABLE 19
Efficacy of nootkatone for repelling cat fleas.

| | Control¹ | | | Treated¹ | | | |
|---|---|---|---|---|---|---|---|
| | # | Isopro- | Un- | # | | Un- | |
| Time | Cat | panol | treat- | Cat | 10% | treat- | Percent |
| (hr) | Fleas | control | ed | Fleas | Nootkatone | ed | Repellency |
| 0.5 | 10 | 4.0 | 6.0 | 10 | 5.6 | 4.4 | −40.0% |
| 1 | 10 | 4.2 | 5.8 | 10 | 3.8 | 6.2 | 9.5% |
| 1.5 | 10 | 4.2 | 5.8 | 10 | 3.8 | 6.2 | 9.5% |
| 2 | 10 | 4.0 | 6.0 | 10 | 3.2 | 6.8 | 20.0% |
| 24 | 10 | 3.6 | 6.4 | 10 | 2.2 | 7.8 | 38.9% |

¹Each result presented in this table is the average cat flea count of five trials.
² Nootkatone repellency was corrected to account for repellency observed for the control group (isopropanol alone) as described in the text.

The results from the experiment above indicated that nootkatone, at 10% in isopropyl alcohol, does not repel cat fleas.

### Example 5

### Immobilization and Killing of Body Lice by 10% Nootkatone

A shampoo with 10% nootkatone formulation was evaluated for efficacy against body lice, as described in the American Society for Testing and Materials (ASTM) protocol E938-94, “Standard Test Method for Determining the Effectiveness of Liquid, Gel, Cream, or Shampoo Insecticides Against Adult Human Lice.” The shampoo was prepared using 80% Just the Basics Shampoo (which contains water, sodium laureth sulfate, cocamide MEA, cocamidopropyl betaine, glycerin, tocopheryl acetate, panthenol, sodium methyl cocoyl taurate, PEG-7 glyceryl cocoate, polyquaternium-10, PPG-12-buteth-16, polyquaternium-7, citric acid, sodium chloride, disodium EDTA, tetrasodium EDTA, methylchloroisothiazolinone, methylisothiazolinone, and fragrance), 10% nootkatone; 5% ethyl alcohol; 5% isopropyl alcohol. Nootkatone-containing shampoo was tested for knockdown (the inability to move toward heat: sickly, but not necessarily dying) 1 hour after treatment and mortality 24 hours after treatment. Briefly, for each of 5 replicates, 25 adult lice of both genders were placed in a 9 dram vial, equipped with a screen plunger to keep the lice from floating to the surface. The vial was then submerged in the treatment formulation in a 100-ml beaker, pre-equilibrated in a 32° C. water bath, for 10 minutes.

Afterwards the lice were washed in the 9 dram vial with distilled water warmed to 32° C. for 1 minute, twice. Excess water was removed by blotting. The lice were then transferred to a clean 4×4 cm patch of dark corduroy cloth, which was placed in a Petri dish in an incubator maintained at 31.7° C. and 60% relative humidity. 1 hour post treatment, the patch was removed and placed on top of a second patch, in a Petri dish, on a slide warmer set at 37.1° C. Lice incapable of moving to the lower (warmer) patch within 5 minutes were considered to be knocked down. The Petri dish containing the corduroy patch holding lice capable of moving toward heat was then returned to the incubator until mortality was assessed at 24 hours post treatment. At 24 hours, lice were categorized as alive (alive and able to move toward heat), moribund (alive but unable to move toward heat), or dead. Negative control experiments were conducted using 10% isopropanol in water in place of the 10% nootkatone containing shampoo. For mortality studies, dead and moribund lice were summed and divided by the number of lice to give mortality at 24 hours. Mortality counts for the nootkatone-treatment group was corrected to mean corrected percent using Abbott's Formula to take into account mortality caused by 10% isopropanol control treatment (see correction formula in Examples 2 and 4 above).

Knockdown results at 1 hour post treatment are set forth in Table 20 below; and mortality results for control (10% isopropanol) and 10% nootkatone in shampoo base at 24 hours are set forth in Tables 21 and 22 below, respectively.

TABLE 20
10% nootkatone-induced knockdown of adult body lice at 1 hour

| | # | | | | Knockdown |
|---|---|---|---|---|---|
| Treatment | Lice¹ | Alive¹ | Knockdown¹ | Dead¹ | (%) |
| 10% Isopropanol | 25 | 25 | 0 | 0 | 0% |
| control | | | | | |
| 10% nootkatone | 24.4 | 0 | 24.4 | 0 | 100% |
| in shampoo base | | | | | |

¹Each result presented is the average body lice count of 5 trials.

TABLE 21
24 hour body lice mortality of 10% Isopropanol control group

| | Total # | | | | Isopropanol |
|---|---|---|---|---|---|
| Rep # | Lice | Alive | Moribund | Dead | control mortality |
| 1 | 25 | 25 | 0 | 0 | 0% |
| 2 | 25 | 24 | 0 | 1 | 4% |
| 3 | 25 | 22 | 0 | 3 | 12% |
| 4 | 25 | 25 | 0 | 0 | 0% |
| 5 | 25 | 25 | 0 | 0 | 0% |
| Average: | 25 | 24.2 | 0 | 0.8 | 3.2% |

TABLE 22
24-hour body mortality of 10% in shampoo base treatment group

| | | | | | Isopropanol | | Corrected |
|---|---|---|---|---|---|---|---|
| | Total # | | | | control | Treatment | Treatment |
| Rep # | Lice | Alive | Moribund | Dead | mortality¹ | mortality | mortality² |
| 1 | 25 | 2 | 6 | 17 | 3.2% | 92.0% | 91.7% |
| 2 | 24 | 2 | 0 | 22 | 3.2% | 91.7% | 91.4% |
| 3 | 25 | 3 | 6 | 16 | 3.2% | 88.0% | 87.6% |
| 4 | 24 | 2 | 9 | 13 | 3.2% | 91.7% | 91.4% |
| 5 | 24 | 4 | 2 | 18 | 3.2% | 83.3% | 82.8% |
| Average: | 24.4 | 2.6 | 4.6 | 17.2 | 3.2% | 89.3% | 89.0% |

¹Values used for isopropanol control mortality were obtained from the average mortality for the 10% isopropanol treatment group in Table 12 above.
²Treatment mortality was corrected to account for mortality observed for the control group (isopropanol alone) as described in the text.

In summary, 10% nootkatone in shampoo base caused 100% knockdown and 89% mortality of adult lice.

It is contemplated to compositions containing at least 0.75%, 1%, 2%, 3%, 4%, 5%, 6%, 7%, 8%, 9%, 10%, greater than 10%, greater than 15%, greater than 20%, or greater than 25% nootkatone and/or an analog of nootkatone, can be used in the exemplified embodiments and all embodiments described herein.

Since modifications will be apparent to those of skill in this art, it is intended that this invention be limited only by the scope of the appended claims.

1. A method of repelling bedbugs, comprising deploying an insect repellent composition, whereby the bedbug is repelled when it comes into contact with the composition or vapors from the composition, wherein the composition comprises greater than 10% by weight nootkatone and/or a derivative or analog of nootkatone in a carrier in an amount up to, but not including 90% by weight.

2. The method of claim 1, wherein the composition is deployed by:
applying the composition topically to an article of clothing of a human; or
applying the composition topically to skin or hair of a human; or
applying the composition topically to skin or fur of an animal; or
laundering an article of clothing of a human with a detergent or fabric softener or both that comprises the composition; or
drying an article of clothing of a human with a fabric softener that comprises the composition; or
applying the composition to bedding, bed boards, bed slats, a mattress, box springs, furniture, carpeting, baseboards or flooring or a combination thereof; or
spraying the composition on to the surface of bedding, bed boards, bed slats, a mattress, box springs, furniture or carpeting; or
injecting the composition into the mattress, box springs, furniture or carpeting or a combination thereof; or
deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or
injecting the composition into a wall space.

3. An insect repellant or pesticide composition, comprising: greater than 15% by weight nootkatone and/or derivative or analog thereof;
a carrier in an amount up to, but not including 85% by weight, wherein the carrier and composition are formulated for deploying or applying the composition directly to an animal or a surface, wherein the carrier comprises:
one or more of diethyl ether, isopropyl ether, n-propyl ether, ethylene glycol, ethylene glycol monomethyl ether, ethylene glycol dimethyl ether, methylene glycol, methylene glycol monomethyl ether, methylene glycol dimethyl ether, propylene glycol, propylene glycol monomethyl ether, propylene glycol dimethyl ether, butylene glycol, butylene glycol monomethyl ether, butylene glycol dimethyl ether, acetone, a methyl ketone, a methyl benzyl ketone, a methyl ethyl ketone, a methyl isopropyl ketone, a methyl butyl ketone, an ethyl ketone, benzyl methyl ketone, alumina, amorphous silica, attapulgite, calcium carbonate, calcium phosphate, clay, chalk, fumed silica, diatomaceous earth, a kaolin, magnesium carbonate, microparticulate cellulose, montmorillonite, pyrophyllite, silicic acid, sodium bicarbonate, sodium carbonate, sodium phosphate, sodium pyrophosphate, talc vermiculite, 1-propanol, 2-propanol, 1-butanol, 2-butanol, 2-methyl-1-propanol, 1-pentanol, 2-pentanol, 3-methyl-1-butanol, 3-methyl-2-butanol, ethylene glycol, propylene glycol, 1,4-butanediol, 1,3-butanediol, 2-methyl-1,3-propanediol, 1,4-cyclohexanedimethanol, diethylene glycol, triethylene glycol, PEG-200, PEG-300, PEG-400, PEG-600, 2-methoxyethanol, 2-ethoxyethanol, 2-propoxyethanol, 2-isopropoxyethanol, 2-butoxyethanol, 1-methoxy-2-propanol, 1-ethoxy-2-propanol, 3-methoxy-1-butanol, diethylene glycol monomethyl ether, diethylene glycol monoethyl ether, diethylene glycol mono-n-propyl ether, diethylene glycol mono-isopropyl ether, diethylene glycol monobutyl ether, triethylene glycol monomethyl ether, glycerol, 3-methoxy-1,2-propanediol, or 3-ethoxy-1,2-propanediol, borneol, citronellol, geraniol, D-limonene dipentene and an aerosol propellant; and/or
a particulate selected from among an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, and vermiculite.

4. The composition of claim 3, wherein
the carrier comprises water, an alcohol, an aldehyde, an alkane, an alkene, an amide, an amine, a diglyceride, an ester, an ether, a glycol ether, a fat, a fatty acid, a glycol ester, a ketone, lanolin, mineral oil, a monoglyceride, paraffin oil, a polyethylene glycol, petrolatum, a propylene carbonate, silicone, tall oils, a terpene hydrocarbon, a terpene alcohol, a triglyceride, finely divided organic solid material, finely divided inorganic solid materials, or mixtures thereof.

5. (canceled)

6. The composition of claim 3, wherein the carrier comprises diethyl ether, isopropyl ether, n-propyl ether, or a combination thereof.

7.-11. (canceled)

12. The composition of claim 3, wherein the carrier comprises an aerosol propellant, and is provided as sprayable propellant pressurized aerosol.

13. The composition of claim 3, wherein the derivative or analog of nootkatone is selected from among nootkatone-11,12-epoxide, nootkatone-1,10-epoxide, nootkatone-1,10-11,12-diepoxide, tetrahydronootkatone and 1,10-dihydronootkatone and combinations thereof.

14. The composition of claim 3, wherein the the nootkatone and/or derivative or analog thereof is present in an amount up to and including 20% or up to and including 25% by weight of the composition.

15.-37. (canceled)

38. The composition of claim 3, wherein the composition is formulated as a personal care or cosmetic composition.

39. The composition of claim 38, wherein the personal care or cosmetic composition is formulated as a product selected from among insect repellents, skin care products, hair care products, and cleansing products.

40.-42. (canceled)

43. The composition of claim 3 formulated as a household care composition.

44. The composition of claim 3 that is formulated as a product selected from among air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles.

44. (canceled)

45. The composition of claim 44 that is a fabric softening composition selected from among a liquid fabric softener, a fabric softening rinse, a fabric softening sheet, and a fabric softening gel.

46.-52. (canceled)

53. An insect repellent or pesticide composition, comprising:
greater than 10% by weight nootkatone and/or a derivative or analog of nootkatone; and
a carrier in an amount up to, but not including 90% by weight, wherein:
the carrier is a substrate that is a paper, a cloth or a woven or nonwoven material and the carrier comprises 0.1 to 5% gelling agent selected from among agar, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, κ-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof.

54.-63. (canceled)

64. A fabric treatment sheet comprising the composition of claim 53.

65.-66. (canceled)

67. A moist towelette product, comprising:
a woven or non-woven fabric or cellulosic substrate; and
a composition of claim 3.

68.-72. (canceled)

73. A method of repelling an insect from a location, comprising:
deploying a composition of claim 3 to the location, whereby the insect is repelled when the insect comes into contact with the composition or vapors from the composition.

74.-76. (canceled)

77. A method of repelling an insect from clothing or bedding, comprising:
deploying a composition to the location whereby the insect is repelled when the insect comes into contact with the composition or vapors from the composition, wherein:
the composition comprises greater than 10% by weight nootkatone and/or a derivative or analog of nootkatone in a carrier in an amount up to, but not including 90% by weight; and the composition is deployed by:
applying topically to an article of clothing or bedding; or
laundering the article of clothing or bedding with a detergent or fabric softener or both that comprises the composition, or
drying the article of clothing or bedding with a fabric softener that comprises the composition.

78.-88. (canceled)

89. A method for killing an insect or pest, comprising:
providing a composition of claim 3; and
applying the composition to the insect or pest, whereby the insect or pest is killed, wherein the amount applied is sufficient to kill the insect or pest.

90. A method of treating a structure infested with termites, comprising:
deploying a composition to the infested structure, whereby the termites are killed and/or repelled, wherein the composition comprises greater than 10% by weight nootkatone and/or a derivative or analog of nootkatone in a carrier in an amount up to, but not including 90% by weight.

91. A method of treating lice, comprising contacting an affected body part containing lice with a composition that comprises at least 0.1% nootkatone or an analog thereof.

92.-98. (canceled)

99. A method for treating a subject infested with chiggers or mites, comprising:
providing a composition containing at least 0.1% nootkatone or an analog thereof; and
applying the composition to a surface of the subject, wherein the chiggers or mites are repelled from the surface or die after coming into contact with the composition or with vapors from the composition.

100.-102. (canceled)

103. A composition, comprising:
greater than 10% to at least about 25%, by weight, nootkatone or an analog thereof; and
an active ingredient selected from among N,N-diethyl-meta-toluamide (DEET), picaridin (2-(2-hydroxyethyl)-1-piperidinecarboxylic acid 1-methylpropyl ester), citronella oil, camphor oil, cedarwood oil, coumarin, 2-hydroxy-methylcyclohexyl acetic acid lactone, beta-alanine, 2-hydroxymethyl-cyclohexylidene acetic acid lactone, 2-hydroxy-methylcyclohexyl propionic acid lactone, p-menthane-3,8-diol, and 3-[N-butyl-N-acetyl]-aminopropionic acid ethyl ester and combinations thereof.

104.-106. (canceled)
